(19) United States
(12) Patent Application Publication
(10) Pub. No.: US 2023/0173103 A1
(43) Pub. Date: Jun. 8, 2023

(54) COMPOSITIONS AND METHODS FOR TREATMENT OF FAMILIAL HYPERCHOLESTEROLEMIA AND ELEVATED LOW-DENSITY LIPOPROTEIN CHOLESTEROL

(71) Applicant: Saliogen Therapeutics, Inc., Cambridge, MA (US)

(72) Inventors: Joseph J. HIGGINS, Cambridge, MA (US); Scott MCMILLAN, Cambridge, MA (US); Ray TABIBIAZAR, Cambridge, MA (US)

(21) Appl. No.: 17/922,118

(22) PCT Filed: Apr. 29, 2021
(86) PCT No.: PCT/US2021/030006

§ 371 Date: Oct. 28, 2022

Related U.S. Application Data
(60) Provisional application No. 63/017,424, filed on Apr. 29, 2020.

(51) Int. Cl. A61K 48/00; C07K 14/705; C12N 15/113; A61K 45/06; A61K 38/45; C12N 15/85; C12N 9/12

(52) U.S. Cl. CPC A61K 48/0058; A61K 38/45; A61K 45/06; C07K 14/705; C12N 9/1241; C12N 15/85; C12N 15/1137; C12N 2310/141; C12N 2800/107

(57) ABSTRACT

Gene therapy compositions and methods are provided for targeting a very low-density lipoprotein receptor gene (VLDLR) and a low-density lipoprotein receptor gene (LDLR) to lower total cholesterol and/or low-density lipoprotein cholesterol (LDL-C) in a patient, thereby treating or mitigating familial hypercholesterolemia.

### FIELD OF THE INVENTION

The present invention relates, in part, to methods, compositions, and products for treating and/or mitigating diseases and conditions related to elevated low-density lipoprotein cholesterol (LDL-C).

### PRIORITY

The present application claims priority to and benefit from the U.S. Provisional Pat. Application No. 63/017,424 filed Apr. 29, 2020, the entirety of which is incorporated by reference herein.

### DESCRIPTION OF THE TEXT FILE SUBMITTED ELECTRONICALLY

This application contains a Sequence Listing in ASCII format submitted electronically herewith via EFS-Web. The ASCII copy, created on Apr. 28, 2021, is named SAL-001PC_Sequence Listing_ST25.txt and is 75,515 bytes in size. The Sequence Listing is incorporated herein by reference in its entirety.

### BACKGROUND

Familial hypercholesterolemia (FH) is an autosomal dominant disease that is one of the most frequent dyslipidemias and is characterized by high concentrations of total and low-density lipoprotein cholesterol (LDL-C) since birth. It is a life-threatening condition with a frequency between 1:200-1:250 persons, which leads to accelerated atherosclerosis and premature coronary heart disease. See Hopkins et al., J Clin Lipidol 2011;5:S9-17; Talmud et al. Curr Opin Lipidol 2014;25:274-81.

Cholesterol is an essential cellular component and the maintenance of cholesterol homeostasis is critical for normal physiological functions. Elevated levels of plasma cholesterol are associated with various pathological conditions, most notably coronary heart disease where high cholesterol levels lead to foam cell formation and plaque buildup in arteries, potentially resulting in a heart attack or stroke. Regulation of cellular cholesterol metabolism and plasma cholesterol levels depends on low-density lipoprotein (LDL) receptor-mediated LDL uptake into specific cells. LDL is the major carrier of cholesterol in the blood, accounting for more than 60% of total plasma cholesterol. LDL is taken up by hepatic and extra-hepatic tissues through receptor mediated endocytosis triggered by apoB-100-LDL receptor interaction. The internalized LDL particle is transported to lysosomes where it is degraded to free cholesterol and amino acids. In humans, the liver is the most important organ for LDL catabolism and LDL receptor activity. In the liver, LDL can be regulated by pharmacologic intervention. The mechanism underlying the uptake of LDL by hepatic tissue is not clearly understood. Thus, LDL uptake and its regulation are important therapeutic targets for atherosclerosis and related diseases.

FH constitutes one of the most serious commonly inherited metabolic diseases. Despite its high prevalence, FH is still severely underdiagnosed and undertreated. An estimated 70% to 95% of FH results from a heterozygous pathogenic variant in one of three genes (APOB, LDLR, proprotein convertase subtilisin kexin 9 (PCSK9)). Mutations in the low-density lipoprotein receptor gene (LDLR) cause more than 90% of cases. LDLR is a cell surface protein receptor involved in the endocytosis of low-density lipoprotein-cholesterol (LDL-C). After LDL-C is bound at the cell membrane, it is taken up by the cell and transported to lysosomes where the protein moiety is degraded and the cholesterol molecule suppresses further cholesterol synthesis via negative feedback. Pathogenic variants in the LDLR gene either reduce the number of LDL receptors produced within the cells or disrupt the ability of the receptor to bind LDL-C. Homozygous FH (HoFH) and heterozygous FH (HeFH) pathogenic variants in the LDLR gene cause high levels of plasma LDL-C and atherosclerosis.

Homozygous FH results from biallelic (homozygous or compound heterozygous) pathogenic variants in one of three genes (APOB, LDLR, PCSK9). HoFH, initially described to affect 1:1,000,000, is now estimated to have a prevalence of 1:160,000 to 1:250,000. Cuchel et al. Eur Heart J 2014;35:2146-57. Most individuals with HoFH experience severe coronary artery disease (CAD) by their mid-20s. The rate of either death or coronary bypass surgery by the teenage years is high, and severe aortic stenosis is also common.

Conventional treatment of FH typically involves multiple pharmacologic agents. For example, statin medications may help HeFH patients, but, in particular for the individuals with HoFH, the response is often attenuated and can be inadequate. Statins can be relatively ineffective in the treatment of HoFH because their efficacy largely depends on the upregulation of functional LDL receptors in the liver. Also, some patients have statin intolerance. In individuals with HoFH, activity of both copies of the LDL receptor are absent or greatly reduced, and therapy for HoFH often requires LDL apheresis (mechanical filtration) in addition to the use of multiple other medications. LDL apheresis is often required starting from a young age, and its use is exacerbated by a limited number of facilities that offer this procedure. In some severe cases, FH patients undergo liver transplantation.

Several medications for treatment of FH and patients with elevated LDL-C exist, including lomitapide, mipomersen (KYNAMRO), and Proprotein Convertase Subtilisin/Kexin Type 9 (PCSK9) inhibitors (e.g., inclisiran, evolocumab (REPATHA), alirocumab (PRALUENT)). However, despite current efforts, LDL-C levels and associated cardiovascular morbidity and mortality remain high in FH patients. FH often requires life-long lipid-lowering drug therapy, which is a burden on patients and on the healthcare system.

Accordingly, there remains a need for improved compositions and methods for lowering LDL-C in patients, thereby improving patients cardiovascular health.

### SUMMARY OF THE INVENTION

The present invention provides compositions and methods for treating and/or mitigating cardiovascular events in patients with familial hypercholesterolemia (FH) or elevated LDL-C. The compositions and methods of the present invention make use of gene transfer constructs comprising transposon expression vectors that use sequence- or locus-specific transposition (SLST) to correct lipid metabolism in patients with LDLR gene mutations or elevated LDL-C. In this way, the present invention provides ways for restoring the LDL metabolism in hepatocytes, in homozygous FH (HeFH), heterozygous FH (HoFH) patients, and patients with elevated LDL-C. The present invention can be used to treat familial hypercholesterolemia, common hypercholesterolemia, increased triglycerides, insulin resistance, metabolic syndrome and other diseases characterized by elevated levels of total cholesterol.

Accordingly, in some aspects, a composition is provided that comprises a gene transfer construct, comprising (a) a nucleic acid encoding a very low-density lipoprotein receptor (VLDLR) protein or a low-density lipoprotein receptor (LDLR) protein or a functional fragment thereof; (b) a liver-specific promoter; and (c) a non-viral vector comprising one or more transposase recognition sites and one or more inverted terminal repeats (ITRs) or end sequences. In embodiments, these constructs can be combined with a PCSK9 miRNA to reduce hepatic synthesis of PCSK9 and upregulate both LDL and VLDL receptors. The gene transfer allows for stable site-specific genomic integration of a functional VLDLR or LDLR. The gene transfer construct of the present invention allows lowering total cholesterol level to thereby treat and/or mitigate FH and its symptoms, and improve overall cardiovascular health of a patient.

The gene therapy in accordance with the present disclosure can be performed using transposon-based vector systems, with the assistance by transposases, which are provided on the same vector (cis) as the gene to be transferred or on a different vector (trans). The transposon-based vector systems can operate under control of a liver-specific promoter. In some embodiments, the liver-specific promoter is an LP1 promoter. The LP1 promoter can be a human LP1 promoter, which can be constructed as described, e.g., in Nathwani et al. Blood vol. 107(7) (2006):2653-61, which is incorporated herein by reference in its entirety. In embodiments, the LP1 promoter is described in in Nathwani et al. Blood vol. 107(7) (2006):2653-61, which is incorporated herein by reference in its entirety, see, e.g. Figure S1 of Nathwani et al.

In embodiments, the transposase, e.g. one derived from Bombyx mori, Xenopus tropicalis, Trichoplusia ni, Rhinolophus ferrumequinum, Rousettus aegyptiacus, Phyllostomus discolor, Myotis myotis, Myotis lucifugus, Pteropus vampyrus, Pipistrellus kuhlii, Pan troglodytes, Molossus molossus, or Homo sapiens, and/or is an engineered version thereof, is used to insert the VLDLR or LDLR gene with or without a PCSK9 silencing miRNA, of the gene transfer construct into a patient’s genome (hence, the VLDLR or LDLR gene construct, with or without the PCSK9 silencing miRNA, is occasionally referred to herein as a transposon).

In embodiments, a transposase is a Myotis lucifugus transposase (MLT, or MLT transposase), which comprises an amino acid sequence of SEQ ID NO: 9, or a variant having at least about 90%, or at least about 93%, or at least about 95%, or at least about 97%, or at least about 98%, or at least about 99% identity thereto, and one or more mutations selected from L573X, E574X, and S2X, wherein X is any amino acid or no amino acid, optionally X is A, G, or a deletion. In embodiments, the mutations are L573del, E574del, and S2A.

In embodiments, an MLT transposase has one or more mutations selected from L573X, E574X, and S2X. In some embodiments, X is any amino acid or no amino acid.

In embodiments, the MLT transposase comprises an amino acid sequence with mutations L573del, E574del, and S2A, and additionally with one or more mutations that confer hyperactivity (or hyperactive mutations). In embodiments, the hyperactive mutations are one or more of S8X, C13X, and N125X mutations, wherein X is optionally any amino acid or no amino acid, optionally X is P, R, or K. In embodiments, the mutations are S8P, C13R, and N125K. In some embodiments, the MLT transposase has S8P and C13R mutations. In some embodiments, the MLT transposase has N125K mutation. In some embodiments, the MLT transposase has all three S8P, C13R, and N125K mutations.

The described compositions can be delivered to a host cell using lipid nanoparticles (LNPs). In some embodiments, the LNP comprises one or more molecules selected from a neutral or structural lipid (e.g. DSPC), cationic lipid (e.g. MC3), cholesterol, PEG-conjugated lipid (CDM-PEG), and a targeting ligand [(e.g. N-Acetylgalactosamine (GalNAc)]. In some embodiments, the LNP comprises GalNAc or another ligand for Asialoglycoprotein Receptor (ASGPR)-mediated uptake into hepatocytes with decreased or absent LDL, VLDL or other lipid receptors.

In some embodiments, a method for lowering total cholesterol and/or LDL-C in a patient is provided, which can be an in vivo or ex vivo method. Accordingly, in some embodiments, a method is provided that comprises administering to a patient in need thereof a composition in accordance with embodiments of the present disclosure. In some embodiments, an ex vivo method for lowering total cholesterol and/or LDL-C in a patient comprises (a) contacting a cell obtained from a patient with the described composition, and (b) administering the cell to a patient in need thereof.

In some embodiments, a method for treating and/or mitigating elevated LDL-C is provided, which can also be performed in vivo or ex vivo. The method for treating and/or mitigating elevated LDL-C can comprise treating and/or mitigating FH. In some embodiments, the method comprises administering to a patient in need thereof composition in accordance with embodiments of the present disclosure. In some embodiments, a method for treating and/or mitigating FH comprises (a) contacting a cell obtained from a patient with a composition of the present disclosure, (b) administering the cell to a patient in need thereof.

The compositions and methods in accordance with embodiments of the present disclosure are substantially non-immunogenic, do not cause any unmanageable side effects, and, in some cases, can be effectively delivered via a single administration. The treatment and/or mitigation of FH or the lowering of total cholesterol and/or LDL-C can be robust and durable. The described compositions and methods allow treating and/or mitigating coronary artery disease (CAD) or atherosclerosis.

In some aspects of the present disclosure, an isolated cell is provided that comprises the composition in accordance with embodiments of the present disclosure.

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs.

### BRIEF DESCRIPTION OF DRAWINGS

FIG. 1 depicts a schematic representation of the human LDRL gene.

FIG. 2 depicts a schematic representation of the human LDRL gene mRNA.

FIG. 3 depicts a schematic representation of the human LDRL protein.

FIGS. 4A, 4B, 4C, 4D, 4E, 4F, 4G, and 4H are schematic representations of gene transfer constructs used in some embodiments of the present disclosure.

FIG. 5 illustrates the lipid nanoparticle structure used in some embodiments of the present disclosure.

FIG. 6 illustrates GFP expression of Huh7 cells 24 hours post nucleofection; the four panels (from left to right) illustrate data obtained with Nucleofector® Solution kit (Lonza, Basel, Switzerland), Programs CA-137, CM-150, CM-138, and EO-100. The top row of each panel shows GFP fluorescence, and the bottom row shows brightfield microscopy data.

FIG. 7 illustrates results of GFP expression of Huh7 cells 4 days post nucleofection; the four panels (from left to right) illustrate data obtained with Nucleofector® Solution kit, Programs CA-137, CM-150, CM-138, and EO-100. The top row of each panel shows GFP fluorescence, and the bottom row shows brightfield microscopy data.

FIGS. 8A, 8B, 8D, and 8D illustrate results of quantification of nucleofection efficiency 24 hours post nucleofection: data obtained with Nucleofector® Solution kit, Programs CA-137 (FIG. 8A), CM-150 (FIG. 8B), CM-138 (FIG. 8C), and EO-100 (FIG. 8D). The top row of each figure shows GFP fluorescence, and the bottom row shows fluorescent-activated cell sorting (FACS) plots.

FIGS. 9A, 9B, 9C, 9D, 9E, 9F, and 9G illustrate FACS plots of transfected Huh7 cells under different conditions, 7 days post transfection, as follows: (FIG. 9A) no program - negative assay control; (FIG. 9B) pmaxGFP - positive assay control; (FIG. 9C) LP1-VLDLR/PGK-GFP plasmid (i.e. a plasmid including a LP1 promoter, a very low-density lipoprotein receptor (VLDLR) gene, and PGK promoter-driven expression of enhanced (eGFP)); (FIG. 9D) LP1-VLDLR/PGK-GFP + MLT 1; (FIG. 9E) LP1-VLDLR/PGK-GFP + MLT 2; (FIG. 9F) MLT 1; and (FIG. 9G) MLT 2.

FIG. 10 depicts LDL-C uptake of untreated Huh7 cells vs. Huh7 cells treated with the VLDLR plasmid and the MLT transposase 2.

FIG. 11 depicts RT qPCR results of VLDLR expression under varying conditions: untreated Huh7 cells; Huh7 cells treated with the LP1-hVLDRL donor and MLT transposase; Huh7 cells treated with the LP1-hVLDRL + PGK-GFP donor and MLT transposase; Huh7 cells do not treated with a donor (“NO DONOR”); Huh7 cells treated with donor (LP1-hVLDRL) only “DONOR ONLY”; Huh7 cells treated with donor (LP1-hVLDRL + PGK-GFP) only “DONOR ONLY”; HEK293 cells.

FIG. 12 is a schematic representation of a gene transfer construct (DNA donor plasmid) used in some embodiments of the present disclosure.

FIGS. 13A, 13B, and 13C are images and FACS plots of GFP expression in Huh7 cells, for assays with (“transposase+”) and without (“transposase-”) the transposase (MLT 1 and MLT 2), which used the gene transfer construct of FIG. 12. FIGS. 13A, 13B, and 13C show data for transposase- assay (top) and transposase+ assay (bottom) at 24 hours (FIG. 13A), 72 hours (FIG. 13B), and 7 days (FIG. 13C), respectively.

FIGS. 14A, 14B, 14C, and 14D illustrate baseline LDL uptake in non-nucleofected (untreated) HEK293 and Huh7 cells: FIG. 14A shows untreated HEK293 cells, LDL uptake only; FIG. 14B shows untreated Huh7 cells, LDL uptake only; FIG. 14C shows untreated HEK293 cells, unstained; and FIG. 14D shows untreated Huh7 cells, unstained.

FIGS. 15A, 15B, 15C, 15D, and 15E illustrate LDL uptake in transposed versus non-transposed Huh7 cells: FIG. 15A shows, for comparison, untransposed (transposase -) HEK293 cells; FIG. 15B shows untransposed (transposase -) Huh7 cells; FIG. 15C shows untransposed (transposase -) Huh7 cells, nucleofected with the human LP1-VLDLR plasmid (pVLDLR); FIG. 15D shows transposed with MLT 1 Huh7 cells, nucleofected with the human LP1-VLDLR plasmid (pVLDLR); and FIG. 15E shows transposed with MLT transposase 2 Huh7 cells, nucleofected with the human LP1-VLDLR plasmid (pVLDLR).

FIGS. 16A and 16B show whole body bioluminescence imaging (BLI) for animal cohort 1 (described in Table 7) on day 3 (FIG. 16A) and day 5 (FIG. 16B).

FIGS. 17A and 17B show BLI for animal cohort 2 (described in Table 8) on day 3 (FIG. 17A) and day 5 (FIG. 17B).

FIGS. 18A and 18B show BLI for animal cohort 3 (described in Table 9) on day 3 (FIG. 18A) and day 5 (FIG. 18B).

FIG. 19 shows BLI from animals that belong to animal group 6 and animal group 8 after a long-term maintenance (26 and 33 days, respectively).

FIG. 20 shows: left panel - BLI from the animal serial no. 6001 (Table 8) administered LNP medium dose (250 uL) (Idlr-/-), at day 26; right panel - BLI from the animal serial no. 8002 (Table 9) administered LNP low dose (125 uL) (Idlr-/-), at day 26.

FIG. 21A shows the results of the efficacy assessment of liver-specific LNP formulation of the present disclosure, in Idlr -/- mice. Triglycerides (mg/dL) are shown at the Reference range, and on days 7 (D7) and 15 (D15), for untreated and treated (vldlr) mice.

FIG. 21B shows the results of the safety assessment, as a measurement of Alanine Aminotransferase (ALT) in the blood of the test animals (in u/L). The data is shown for the Untreated, Treated (vLdlr), and PBS groups, each including data for the Reference (“1”) range, and on D7 (“2”), and D15 (“3”).

### DETAILED DESCRIPTION OF THE INVENTION

The present invention is based, in part, on the discovery that non-viral, capsid free gene therapy methods and compositions can be used for lowering of total cholesterol and low density lipoprotein cholesterol (LDL-C), and thereby treating and mitigating the cardiovascular effects of familial hypercholesterolemia (FH) and LDL-C elevations. The non-viral gene therapy methods in accordance with the present disclosure find use in liver-directed gene therapy to correct mutations in a low-density lipoprotein receptor gene (LDLR), very low-density lipoprotein receptor gene (VLDLR), or to mitigate serum elevations of LDL-C. The described methods and compositions employ transposition of LDLR or VLDLR. The described methods and compositions improve cardiovascular health of patients and thereby improve overall prognosis and health of the patients.

In some embodiments, the described methods and compositions can be administered as a one-time dose or repeated doses. The administration route can be, in some embodiments, intravenously or to the intraportal vein or directly to the liver parenchyma.

FH is caused by mutations in three primary genes, LDLR, APOB, and PCSK9, but mutations in LDLR are the most common - more than 90% of reported FH-causing variants are in LDLR, with 5% to 10% in APOB and less than 1% in PCSK9. See lacocca et al. Expert Rev Mol Diagn 2017; 17:641-51.

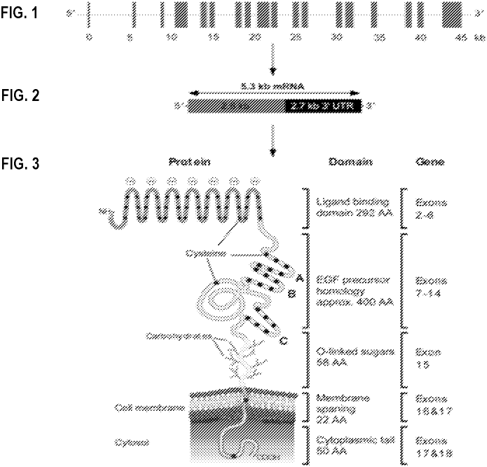

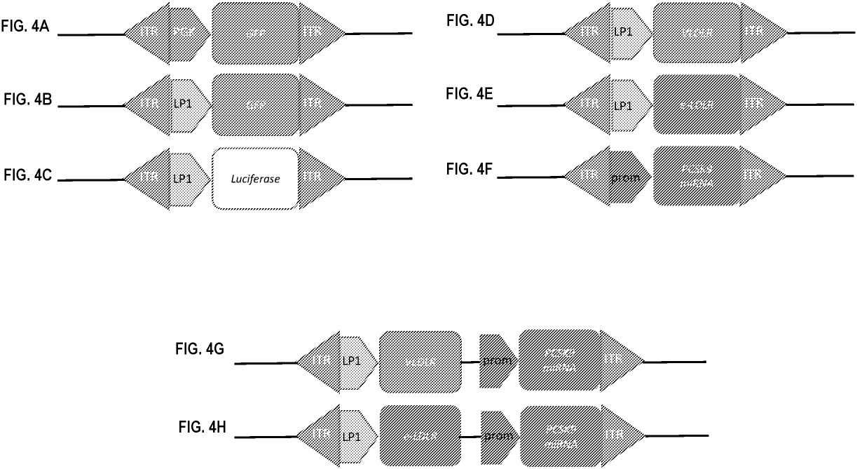

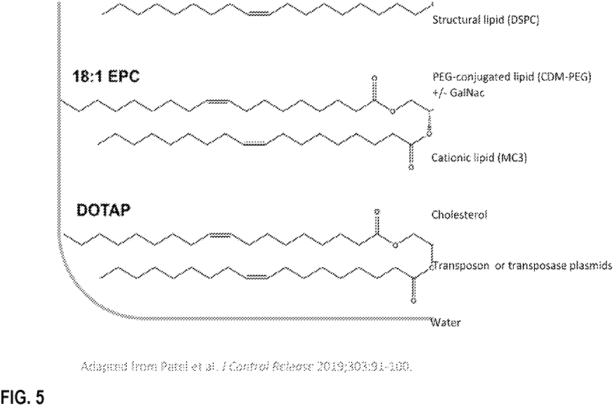

LDLR is located on chromosome 19 and its genomic structure spans 45 kb including 18 exons and 17 introns. The LDLR gene, mRNA, and protein are shown schematically in FIGS. 1, 2, and 3, respectively. The human LDLR transcript variant 1 mRNA (NCBI Reference: NM_000527.5) is:

1 gtgcaatcgc gggaagccag ggtttccagc taggacacag caggtcgtga tccgggtcgg
61 gacactgcct ggcagaggct gcgagcatgg ggccctgggg ctggaaattg cgctggaccg
121 tcgccttgct cctcgccgcg gcggggactg cagtgggcga cagatgcgaa agaaacgagt
181 tccagtgcca agacgggaaa tgcatctcct acaagtgggt ctgcgatggc agcgctgagt
241 gccaggatgg ctctgatgag tcccaggaga cgtgcttgtc tgtcacctgc aaatccgggg
301 acttcagctg tgggggccgt gtcaaccgct gcattcctca gttctggagg tgcgatggcc
361 aagtggactg cgacaacggc tcagacgagc aaggctgtcc ccccaagacg tgctcccagg
421 acgagtttcg ctgccacgat gggaagtgca tctctcggca gttcgtctgt gactcagacc
481 gggactgctt ggacggctca gacgaggcct cctgcccggt gctcacctgt ggtcccgcca
541 gcttccagtg caacagctcc acctgcatcc cccagctgtg ggcctgcgac aacgaccccg
601 actgcgaaga tggctcggat gagtggccgc agcgctgtag gggtctttac gtgttccaag
661 gggacagtag cccctgctcg gccttcgagt tccactgcct aagtggcgag tgcatccact
721 ccagctggcg ctgtgatggt ggccccgact gcaaggacaa atctgacgag gaaaactgcg
781 ctgtggccac ctgtcgccct gacgaattcc agtgctctga tggaaactgc atccatggca
841 gccggcagtg tgaccgggaa tatgactgca aggacatgag cgatgaagtt ggctgcgtta
901 atgtgacact ctgcgaggga cccaacaagt tcaagtgtca cagcggcgaa tgcatcaccc
961 tggacaaagt ctgcaacatg gctagagact gccgggactg gtcagatgaa cccatcaaag
1021 agtgcgggac caacgaatgc ttggacaaca acggcggctg ttcccacgtc tgcaatgacc
1081 ttaagatcgg ctacgagtgc ctgtgccccg acggcttcca gctggtggcc cagcgaagat
1141 gcgaagatat cgatgagtgt caggatcccg acacctgcag ccagctctgc gtgaacctgg
1201 agggtggcta caagtgccag tgtgaggaag gcttccagct ggacccccac acgaaggcct
1261 gcaaggctgt gggctccatc gcctacctct tcttcaccaa ccggcacgag gtcaggaaga
1321 tgacgctgga ccggagcgag tacaccagcc tcatccccaa cctgaggaac gtggtcgctc
1381 tggacacgga ggtggccagc aatagaatct actggtctga cctgtcccag agaatgatct
1441 gcagcaccca gcttgacaga gcccacggcg tctcttccta tgacaccgtc atcagcagag
1501 acatccaggc ccccgacggg ctggctgtgg actggatcca cagcaacatc tactggaccg
1561 actctgtcct gggcactgtc tctgttgcgg ataccaaggg cgtgaagagg aaaacgttat
1621 tcagggagaa cggctccaag ccaagggcca tcgtggtgga tcctgttcat ggcttcatgt
1681 actggactga ctggggaact cccgccaaga tcaagaaagg gggcctgaat ggtgtggaca
1741 tctactcgct ggtgactgaa aacattcagt ggcccaatgg catcacccta gatctcctca
1801 gtggccgcct ctactgggtt gactccaaac ttcactccat ctcaagcatc gatgtcaacg
1861 ggggcaaccg gaagaccatc ttggaggatg aaaagaggct ggcccacccc ttctccttgg
1921 ccgtctttga ggacaaagta ttttggacag atatcatcaa cgaagccatt ttcagtgcca
1981 accgcctcac aggttccgat gtcaacttgt tggctgaaaa cctactgtcc ccagaggata
2041 tggttctctt ccacaacctc acccagccaa gaggagtgaa ctggtgtgag aggaccaccc
2101 tgagcaatgg cggctgccag tatctgtgcc tccctgcccc gcagatcaac ccccactcgc
2161 ccaagtttac ctgcgcctgc ccggacggca tgctgctggc cagggacatg aggagctgcc
2221 tcacagaggc tgaggctgca gtggccaccc aggagacatc caccgtcagg ctaaaggtca
2281 gctccacagc cgtaaggaca cagcacacaa ccacccgacc tgttcccgac acctcccggc
2341 tgcctggggc cacccctggg ctcaccacgg tggagatagt gacaatgtct caccaagctc
2401 tgggcgacgt tgctggcaga ggaaatgaga agaagcccag tagcgtgagg gctctgtcca
2461 ttgtcctccc catcgtgctc ctcgtcttcc tttgcctggg ggtcttcctt ctatggaaga
2521 actggcggct taagaacatc aacagcatca actttgacaa ccccgtctat cagaagacca
2581 cagaggatga ggtccacatt tgccacaacc aggacggcta cagctacccc tcgagacaga
2641 tggtcagtct ggaggatgac gtggcgtgaa catctgcctg gagtcccgtc cctgcccaga
2701 acccttcctg agacctcgcc ggccttgttt tattcaaaga cagagaagac caaagcattg
2761 cctgccagag ctttgtttta tatatttatt catctgggag gcagaacagg cttcggacag
2821 tgcccatgca atggcttggg ttgggatttt ggtttcttcc tttcctcgtg aaggataaga
2881 gaaacaggcc cggggggacc aggatgacac ctccatttct ctccaggaag ttttgagttt
2941 ctctccaccg tgacacaatc ctcaaacatg gaagatgaaa ggggagggga tgtcaggccc
3001 agagaagcaa gtggctttca acacacaaca gcagatggca ccaacgggac cccctggccc
3061 tgcctcatcc accaatctct aagccaaacc cctaaactca ggagtcaacg tgtttacctc
3121 ttctatgcaa gccttgctag acagccaggt tagcctttgc cctgtcaccc ccgaatcatg
3181 acccacccag tgtctttcga ggtgggtttg taccttcctt aagccaggaa agggattcat
3241 ggcgtcggaa atgatctggc tgaatccgtg gtggcaccga gaccaaactc attcaccaaa
3301 tgatgccact tcccagaggc agagcctgag tcactggtca cccttaatat ttattaagtg
3361 cctgagacac ccggttacct tggccgtgag gacacgtggc ctgcacccag gtgtggctgt
3421 caggacacca gcctggtgcc catcctcccg acccctaccc acttccattc ccgtggtctc
3481 cttgcacttt ctcagttcag agttgtacac tgtgtacatt tggcatttgt gttattattt
3541 tgcactgttt tctgtcgtgt gtgttgggat gggatcccag gccagggaaa gcccgtgtca
3601 atgaatgccg gggacagaga ggggcaggtt gaccgggact tcaaagccgt gatcgtgaat
3661 atcgagaact gccattgtcg tctttatgtc cgcccaccta gtgcttccac ttctatgcaa
3721 atgcctccaa gccattcact tccccaatct tgtcgttgat gggtatgtgt ttaaaacatg
3781 cacggtgagg ccgggcgcag tggctcacgc ctgtaatccc agcactttgg gaggccgagg
3841 cgggtggatc atgaggtcag gagatcgaga ccatcctggc taacacgtga aaccccgtct
3901 ctactaaaaa tacaaaaaat tagccgggcg tggtggcggg cacctgtagt cccagctact
3961 cgggaggctg aggcaggaga atggtgtgaa cccgggaagc ggagcttgca gtgagccgag
4021 attgcgccac tgcagtccgc agtctggcct gggcgacaga gcgagactcc gtctcaaaaa
4081 aaaaaaacaa aaaaaaacca tgcatggtgc atcagcagcc catggcctct ggccaggcat
4141 ggcgaggctg aggtgggagg atggtttgag ctcaggcatt tgaggctgtc gtgagctatg
4201 attatgccac tgctttccag cctgggcaac atagtaagac cccatctctt aaaaaatgaa
4261 tttggccaga cacaggtgcc tcacgcctgt aatcccagca ctttgggagg ctgagctgga
4321 tcacttgagt tcaggagttg gagaccaggc ctgagcaaca aagcgagatc ccatctctac
4381 aaaaaccaaa aagttaaaaa tcagctgggt acggtggcac gtgcctgtga tcccagctac
4441 ttgggaggct gaggcaggag gatcgcctga gcccaggagg tggaggttgc agtgagccat
4501 gatcgagcca ctgcactcca gcctgggcaa cagatgaaga ccctatttca gaaatacaac
4561 tataaaaaaa taaataaatc ctccagtctg gatcgtttga cgggacttca ggttctttct
4621 gaaatcgccg tgttactgtt gcactgatgt ccggagagac agtgacagcc tccgtcagac
4681 tcccgcgtga agatgtcaca agggattggc aattgtcccc agggacaaaa cactgtgtcc
4741 cccccagtgc agggaaccgt gataagcctt tctggtttcg gagcacgtaa atgcgtccct
4801 gtacagatag tggggatttt ttgttatgtt tgcactttgt atattggttg aaactgttat
4861 cacttatata tatatatata cacacatata tataaaatct atttattttt gcaaaccctg
4921 gttgctgtat ttgttcagtg actattctcg gggccctgtg tagggggtta ttgcctctga
4981 aatgcctctt ctttatgtac aaagattatt tgcacgaact ggactgtgtg caacgctttt
5041 tgggagaatg atgtccccgt tgtatgtatg agtggcttct gggagatggg tgtcactttt
5101 taaaccactg tatagaaggt ttttgtagcc tgaatgtctt actgtgatca attaaatttc
5161 ttaaatgaac caa (SEQ ID NO: 1)

Pathogenic variants have been reported in the promoter, introns, and exons of LDLR, and the majority of pathogenic variants fall within the ligand-binding (40%) or epidermal growth factor precursor-like (47%) domains, with the highest frequency of pathogenic variants is reported in exon 4 (20%). LDLR encodes a mature protein product of 839 amino acid. LDLR has four distinct functional domains that can function independently of each other: LDL receptor domain class A (LDLa); epidermal growth factor-like domain (EGF); calcium-binding EGF-like domain (EGF-CA); and LDL receptor repeat class B (LDLb). LDLR includes cell surface proteins involved in the endocytosis of LDL cholesterol (LDL-C). After LDL-C is bound at the cell membrane, it is taken into the cell and to lysosomes where the protein moiety is degraded and the cholesterol molecule suppresses cholesterol synthesis via negative feedback.

Pathogenic variants in LDLR usually either reduce the number of LDL receptors produced within the cells or disrupt the ability of the receptor to bind LDL-C. Regardless of the specific mechanism, heterozygous pathogenic variants in LDLR cause high levels of plasma LDL-C.

Hepatic LDLR expression is regulated by PCSK9, a focus of second-generation lipid-lowering drugs that interfere with PCSK9-mediated degradation of the LDLR. Another mechanism involves the post-transcriptional regulation of LDLR expression, based on the degradation of the receptor by the ubiquitin-proteasome pathway, has emerged as an additional target to increase receptor expression. Such strategies to increase LDLR expression are supported by naturally occurring loss-of-function mutations in PCSK9 and the inducible degrader of LDLR (IDOL; MYLIP) that lowers LDL-C levels but have no disease associated with these variants. The human LDLR variants K830R and C839A have been previously reported to prevent IDOL-mediated degradation of LDLR. Somanathan et al., Circ Res 2014; 115:591-9. Additionally, the L339D confers resistance to human PCSK9-mediated degradation. Id. In AAV vectors delivering LDLR containing all three variants (L339D, K830R, and C839A), LDRL was resistant to regulation by both PCSK9 and IDOL pathways as compared to vectors using wild type LDLR. Id.

In some embodiments, the nucleic acid encoding the human LDLR protein, or a functional fragment thereof comprises a nucleotide sequence of NM_000527.5 (SEQ ID NO: 1), or a variant having at least about 90%, or at least about 93%, or at least about 95%, or at least about 97%, or at least about 98% identity thereto.

In embodiments, the LDLR protein is human LDLR protein, or a functional fragment thereof.

In embodiments, the nucleic acid encoding the human LDLR protein, or the functional fragment thereof comprises a nucleotide sequence encoding a protein having an amino acid sequence of SEQ ID NO: 3, or a variant having at least about 90%, or at least about 93%, or at least about 95%, or at least about 97%, or at least about 98% identity thereto.

In embodiments, the nucleic acid encoding the human LDLR protein, or the functional fragment thereof comprises a nucleotide sequence of SEQ ID NO: 1, or a variant having at least about 90%, or at least about 93%, or at least about 95%, or at least about 97%, or at least about 98% identity thereto.

In some embodiments, a human LDLR protein comprises one or more mutations selected from L339D, K830R, and C839A. In some embodiments, the human LDLR comprises a nucleotide sequence of SEQ ID NO: 2 encoding a protein having an amino acid sequence of SEQ ID NO: 3, or a variant having at least about 90%, or at least about 93%, or at least about 95%, or at least about 97%, or at least about 98% identity thereto. In some embodiments, the human LDLR comprises a nucleotide sequence of SEQ ID NO: 2 encoding the amino acid sequence of SEQ ID NO: 3, or a variant having at least about 90%, or at least about 93%, or at least about 95%, or at least about 97%, or at least about 98% identity thereto.

In embodiments, the human LDLR protein, or a functional fragment thereof comprises one or more mutations selected from L18F, K830R, and C839A, with reference to the amino acid sequence of SEQ ID NO: 3.

The nucleotide sequence of SEQ ID NO: 2 (2583 bp) comprises variants L339D, K830L, C839A, which are bolded and underlined below. The amino acid sequence of SEQ ID NO: 3 (860 amino acids) comprises variants L339D, K830L, C839A, which are bolded and underlined below.

SEQ ID NO: 2 is

1 atggggccct ggggctggaa attgcgctgg accgtcgcct tgctcctcgc cgcggcgggg
61 actgcagtgg gcgacagatg cgaaagaaac gagttccagt gccaagacgg gaaatgcatc
121 tcctacaagt gggtctgcga tggcagcgct gagtgccagg atggctctga tgagtcccag
181 gagacgtgct tgtctgtcac ctgcaaatcc ggggacttca gctgtggggg ccgtgtcaac
241 cgctgcattc ctcagttctg gaggtgcgat ggccaagtgg actgcgacaa cggctcagac
301 gagcaaggct gtccccccaa gacgtgctcc caggacgagt ttcgctgcca cgatgggaag
361 tgcatctctc ggcagttcgt ctgtgactca gaccgggact gcttggacgg ctcagacgag
421 gcctcctgcc cggtgctcac ctgtggtccc gccagcttcc agtgcaacag ctccacctgc
481 atcccccagc tgtgggcctg cgacaacgac cccgactgcg aagatggctc ggatgagtgg
541 ccgcagcgct gtaggggtct ttacgtgttc caaggggaca gtagcccctg ctcggccttc
601 gagttccact gcctaagtgg cgagtgcatc cactccagct ggcgctgtga tggtggcccc
661 gactgcaagg acaaatctga cgaggaaaac tgcgctgtgg ccacctgtcg ccctgacgaa
721 ttccagtgct ctgatggaaa ctgcatccat ggcagccggc agtgtgaccg ggaatatgac
781 tgcaaggaca tgagcgatga agttggctgc gttaatgtga cactctgcga gggacccaac
841 aagttcaagt gtcacagcgg cgaatgcatc accctggaca aagtctgcaa catggctaga
901 gactgccggg actggtcaga tgaacccatc aaagagtgcg ggaccaacga atgcttggac
961 aacaacggcg gctgttccca cgtctgcaat gaccttaaga tcggctacga gtgcctg tgc
1021 cccgacggct tccagctggt ggcccagcga agatgcgaag atatcgatga gtgtcaggat
1081 cccgacacct gcagccagct ctgcgtgaac ctggagggtg gctacaagtg ccagtgtgag
1141 gaaggcttcc agctggaccc ccacacgaag gcctgcaagg ctgtgggctc catcgcctac
1201 ctcttcttca ccaaccggca cgaggtcagg aagatgacgc tggaccggag cgagtacacc
1261 agcctcatcc ccaacctgag gaacgtggtc gctctggaca cggaggtggc cagcaataga
1321 atctactggt ctgacctgtc ccagagaatg atctgcagca cccagcttga cagagcccac
1381 ggcgtctctt cctatgacac cgtcatcagc agagacatcc aggcccccga cgggctggct
1441 gtggactgga tccacagcaa catctactgg accgactctg tcctgggcac tgtctctgtt
1501 gcggatacca agggcgtgaa gaggaaaacg ttattcaggg agaacggctc caagccaagg
1561 gccatcgtgg tggatcctgt tcatggcttc atgtactgga ctgactgggg aactcccgcc
1621 aagatcaaga aagggggcct gaatggtgtg gacatctact cgctggtgac tgaaaacatt
1681 cagtggccca atggcatcac cctagatctc ctcagtggcc gcctctactg ggttgactcc
1741 aaacttcact ccatctcaag catcgatgtc aacgggggca accggaagac catcttggag
1801 gatgaaaaga ggctggccca ccccttctcc ttggccgtct ttgaggacaa agtattttgg
1861 acagatatca tcaacgaagc cattttcagt gccaaccgcc tcacaggttc cgatgtcaac
1921 ttgttggctg aaaacctact gtccccagag gatatggttc tcttccacaa cctcacccag
1981 ccaagaggag tgaactggtg tgagaggacc accctgagca atggcggctg ccagtatctg
2041 tgcctccctg ccccgcagat caacccccac tcgcccaagt ttacctgcgc ctgcccggac
2101 ggcatgctgc tggccaggga catgaggagc tgcctcacag aggctgaggc tgcagtggcc
2161 acccaggaga catccaccgt caggctaaag gtcagctcca cagccgtaag gacacagcac
2221 acaaccaccc gacctgttcc cgacacctcc cggctgcctg gggccacccc tgggctcacc
2281 acggtggaga tagtgacaat gtctcaccaa gctctgggcg acgttgctgg cagaggaaat
2341 gagaagaagc ccagtagcgt gagggctctg tccattgtcc tccccatcgt gctcctcgtc
2401 ttcctttgcc tgggggtctt ccttctatgg aagaactggc ggcttaagaa catcaacagc
2461 atcaactttg acaaccccgt ctatcagaag accacagagg atgaggtcca catttgc cac
2521 aaccaggacg gctacagcta cccctcgaga cagatggtca gtctggagga tgacgtggcg
2581 tga (SEQ ID NO: 2)

SEQ ID NO: 3 is

1 MGPWGWKLRW TVALLLAAAG TAVGDRCERN EFQCQDGKCI SYKWVCDGSA ECQDGSDESQ
61 ETCLSVTCKS GDFSCGGRVN RCIPQFWRCD GQVDCDNGSD EQGCPPKTCS QDEFRCHDGK
121 CISRQFVCDS DRDCLDGSDE ASCPVLTCGP ASFQCNSSTC IPQLWACDND PDCEDGSDEW
181 PQRCRGLYVF QGDSSPCSAF EFHCLSGECI HSSWRCDGGP DCKDKSDEEN CAVATCRPDE
241 FQCSDGNCIH GSRQCDREYD CKDMSDEVGC VNVTLCEGPN KFKCHSGECI TLDKVCNMAR
301 DCRDWSDEPI KECGTNECLD NNGGCSHVCN DLKIGYECL C PDGFQLVAQR RCEDIDECQD
361 PDTCSQLCVN LEGGYKCQCE EGFQLDPHTK ACKAVGSIAY LFFTNRHEVR KMTLDRSEYT
421 SLIPNLRNVV ALDTEVASNR IYWSDLSQRM ICSTQLDRAH GVSSYDTVIS RDIQAPDGLA
481 VDWIHSNIYW TDSVLGTVSV ADTKGVKRKT LFRENGSKPR AIVVDPVHGF MYWTDWGTPA
541 KIKKGGLNGV DIYSLVTENI QWPNGITLDL LSGRLYWVDS KLHSISSIDV NGGNRKTILE
601 DEKRLAHPFS LAVFEDKVFW TDIINEAIFS ANRLTGSDVN LLAENLLSPE DMVLFHNLTQ
661 PRGVNWCERT TLSNGGCQYL CLPAPQINPH SPKFTCACPD GMLLARDMRS CLTEAEAAVA
721 TQETSTVRLK VSSTAVRTQH TTTRPVPDTS RLPGATPGLT TVEIVTMSHQ ALGDVAGRGN
781 EKKPSSVRAL SIVLPIVLLV FLCLGVFLLW KNWRLKNINS INFDNPVYQK TTEDEVHIC H
841 NQDGYSYPSR QMVSLEDDVA (SEQ ID NO: 3)

In some embodiments, the nucleic acid encoding the human LDLR may comprise a nucleotide sequence encoding a protein having an amino acid sequence of SEQ ID NO: 3, or a variant having at least about 90%, or at least about 93%, or at least about 95%, or at least about 97%, or at least about 98% identity thereto.

VLDLR is located on chromosome 9 and contains 19 exons spanning approximately 40 kb of the genome. The exon-intron organization of the gene is almost identical to that of the LDLR gene, except for an extra exon that encodes an additional repeat in the ligand binding domain of the VLDL receptor. The Homo sapiens VLDLR transcript (mRNA) variant 1, is 9,213 bp (NCBI Reference: NM_003383.5) or approximately 9.2 kb:

1 accccgctct ccggccgccg ccggtgcggg tgctccgcta ccggctcctc tccgttctgt
61 gctctcttct gctctcggct ccccaccccc tctcccttcc ctcctctccc cttgcctccc
121 ctcctctgca gcgcctgcat tattttctgc ccgcaggctc ggcttgcact gctgctgcag
181 cccggggagg tggctgggtg ggtggggagg agactgtgca agttgtaggg gagggggtgc
241 cctcttcttc cccgctccct tcccccgcca actccttccc ctccttctcc ccctttcccc
301 tccccgcccc caccttcttc ctcctttcgg aaggactggt aacttgtcgt gcggagcgaa
361 cggcggcggc ggcggcggcg gcggcaccat ccaggcgggc accatgggca cgtccgcgct
421 ctgggcgctc tggctgctgc tcgcgctgtg ctgggcgccc cgggagagcg gcgccaccgg
481 aaccgggaga aaagccaaat gtgaaccctc ccaattccag tgcacaaatg gtcgctgtat
541 tacgctgttg tggaaatgtg atggggatga agactgtgtt gacggcagtg atgaaaagaa
601 ctgtgtaaag aagacgtgtg ctgaatctga cttcgtgtgc aacaatggcc agtgtgttcc
661 cagccgatgg aagtgtgatg gagatcctga ctgcgaagat ggttcagatg aaagcccaga
721 acagtgccat atgagaacat gccgcataca tgaaatcagc tgtggcgccc attctactca
781 gtgtatccca gtgtcctgga gatgtgatgg tgaaaatgat tgtgacagtg gagaagatga
841 agaaaactgt ggcaatataa catgtagtcc cgacgagttc acctgctcca gtggccgctg
901 catctccagg aactttgtat gcaatggcca ggatgactgc agcgatggca gtgatgagct
961 ggactgtgcc ccgccaacct gtggcgccca tgagttccag tgcagcacct cctcctgcat
1021 ccccatcagc tgggtatgcg acgatgatgc agactgctcc gaccaatctg atgagtccct
1081 ggagcagtgt ggccgtcagc cagtcataca caccaagtgt ccagccagcg aaatccagtg
1141 cggctctggc gagtgcatcc ataagaagtg gcgatgtgat ggggaccctg actgcaagga
1201 tggcagtgat gaggtcaact gtccctctcg aacttgccga cctgaccaat ttgaatgtga
1261 ggatggcagc tgcatccatg gcagcaggca gtgtaatggt atccgagact gtgtcgatgg
1321 ttccgatgaa gtcaactgca aaaatgtcaa tcagtgcttg ggccctggaa aattcaagtg
1381 cagaagtgga gaatgcatag atatcagcaa agtatgtaac caggagcagg actgcaggga
1441 ctggagtgat gagcccctga aagagtgtca tataaacgaa tgcttggtaa ataatggtgg
1501 atgttctcat atctgcaaag acctagttat aggctacgag tgtgactgtg cagctgggtt
1561 tgaactgata gataggaaaa cctgtggaga tattgatgaa tgccaaaatc caggaatctg
1621 cagtcaaatt tgtatcaact taaaaggcgg ttacaagtgt gaatgtagtc gtggctatca
1681 aatggatctt gctactggcg tgtgcaaggc agtaggcaaa gagccaagtc tgatcttcac
1741 taatcgaaga gacatcagga agattggctt agagaggaaa gaatatatcc aactagttga
1801 acagctaaga aacactgtgg ctctcgatgc tgacattgct gcccagaaac tattctgggc
1861 cgatctaagc caaaaggcta tcttcagtgc ctcaattgat gacaaggttg gtagacatgt
1921 taaaatgatc gacaatgtct ataatcctgc agccattgct gttgattggg tgtacaagac
1981 catctactgg actgatgcgg cttctaagac tatttcagta gctaccctag atggaaccaa
2041 gaggaagttc ctgtttaact ctgacttgcg agagcctgcc tccatagctg tggacccact
2101 gtctggcttt gtttactggt cagactgggg tgaaccagct aaaatagaaa aagcaggaat
2161 gaatggattc gatagacgtc cactggtgac agcggatatc cagtggccta acggaattac
2221 acttgacctt ataaaaagtc gcctctattg gcttgattct aagttgcaca tgttatccag
2281 cgtggacttg aatggccaag atcgtaggat agtactaaag tctctggagt tcctagctca
2341 tcctcttgca ctaacaatat ttgaggatcg tgtctactgg atagatgggg aaaatgaagc
2401 agtctatggt gccaataaat tcactggatc agagctagcc actctagtca acaacctgaa
2461 tgatgcccaa gacatcattg tctatcatga acttgtacag ccatcaggta aaaattggtg
2521 tgaagaagac atggagaatg gaggatgtga atacctatgc ctgccagcac cacagattaa
2581 tgatcactct ccaaaatata cctgttcctg tcccagtggg tacaatgtag aggaaaatgg
2641 ccgagactgt caaagtactg caactactgt gacttacagt gagacaaaag atacgaacac
2701 aacagaaatt tcagcaacta gtggactagt tcctggaggg atcaatgtga ccacagcagt
2761 atcagaggtc agtgttcccc caaaagggac ttctgccgca tgggccattc ttcctctctt
2821 gctcttagtg atggcagcag taggtggcta cttgatgtgg cggaattggc aacacaagaa
2881 catgaaaagc atgaactttg acaatcctgt gtacttgaaa accactgaag aggacctctc
2941 catagacatt ggtagacaca gtgcttctgt tggacacacg tacccagcaa tatcagttgt
3001 aagcacagat gatgatctag cttgacttct gtgacaaatg ttgacctttg aggtctaaac
3061 aaataatacc cccgtcggaa tggtaaccga gccagcagct gaagtctctt tttcttcctc
3121 tcggctggaa gaacatcaag atacctttgc gtggatcaag cttgtgtact tgaccgtttt
3181 tatattactt ttgtaaatat tcttgtccac attctacttc agctttggat gtggttaccg
3241 agtatctgta acccttgaat ttctagacag tattgccacc tctggccaaa tatgcacttt
3301 ccctagaaag ccatattcca gcagtgaaac ttgtgctata gtgtatacca cctgtacata
3361 cattgtatag gccatctgta aatatcccag agaacaatca ctattcttaa gcactttgaa
3421 aatatttcta tgtaaattat tgtaaacttt ttcaatggtt gggacaatgg caataggaca
3481 aaacgggtta ctaagatgaa attgccaaaa aaatttataa actaattttg tacgtatgaa
3541 tgatatcttt gacctcaatg gaggtttgca aagactgagt gttcaaacta ctgtacattt
3601 tttttcaagt gctaaaaaat taaaccaagc agcttaacca tggtttgtgc ctgttcctct
3661 aggatgaact cctatcctga gtagtaccaa gtgttctaac taaatgtgtg agctgtagtt
3721 cttgatgttg tataactaag cctgtaattg ggctggtttc tcttacaccc tagcacagtt
3781 aaaaaatgaa gtactgttta acatttgctc ccgaaatatt tcttactgtg taaaagaagc
3841 tagcttagtc tgtacctaga attcctcttg gttagaggag tgagtttctt ttttttccat
3901 taacttgttt cctgatcgag aaacacgtgc taagatttct atgaattctg cttctttata
3961 gttaagtctc tgttttccat gttctgtcat ttatataggt caaggacaag tgccttctaa
4021 ggcatggata agggctttct tcttatgaaa gtctagactg tcttactgtg aagatggcca
4081 gggaggatag caagcaatgg cattcttgaa cagtctaagg caggtttctc agtgtggtcc
4141 tgagaacaga agcatctgca tcacctggga atgtattaga aatgcaagtt cccagcccca
4201 tcccagacct actgaatcag aaactctggg ggcgagggct gaaacctgct ttcataagcc
4261 cttcaggcga tactgatgta aactaaagct taaaaaccac cgttttactg tgacttcatg
4321 aagaatataa ttgcaatatc acctaaattt ttatcctggc tgggatgggg cagattctta
4381 ctatggacaa tttctggaac ttacagtatg aagagcagat gaggagtttg agacaaagaa
4441 aaaattgtac agggagttta gtgaattttc catcaggttg gccattactt tctttctcta
4501 aagtctcagg atgtctggaa gcaaggaaag acaaaagact tgaagcaccg ggtgcatgct
4561 gtgtgtcaca agtgaggtgg agagttggct gtggaaggta cagcaattcc tctggaatta
4621 aatgaccgac caacacagta ccaagaggat gaagagaaga tgttttgcgt tgctacgtga
4681 atcagtggat gactcatatt tcacttgcat atgaaatata aggcagttaa catcttgttt
4741 gtggcttaag caaggacatt aatcctacta gatgaagggg aagttggttc tgttgtctta
4801 agggatgccc ccttgtgatt ttggtcaact gaatgaaaaa gaaaagatag ccttggactt
4861 ttttttcatg ggaggtttgg aggtcaagca tgtagaagga aaaggctgta gtcatgaata
4921 tagaactgtc tgctctccac cttttcctca ctctacattt agtcatcaca ctgaacagga
4981 tgtggcctgc tgacagtgag tagctgaagt gcctgtttgg taagtaggtt agcattcatc
5041 atgaactctg aaagccattt cagagtttgg ctgctgctct gaccctgctg tggtacaaaa
5101 aacatggatt tttttttttt taactggagt aataccaaat tccctttaga agcttgaagt
5161 ttgatactat tgctttcccc attataagga ttatacagta ttaattaatt ataaggccca
5221 aagtttgttt tggcaaataa agtattattc tctatcatca tcttttgaat tcctgtccct
5281 agtatgttct ttcatgttct gggtaaaatt tccaaccctc aaagagtcta aaacgtctca
5341 ttttttttta ccacgtcctc cccagtaaga agccaaggta caactcattg tacctacgag
5401 gtacaaactc atttgtagaa acaaagtaac tagatattcc tattgaaata agaatagata
5461 tattggtggg ccaggcatgg tggctcacgc ctataatccc agcactttgg aaggccaagg
5521 caggaggatc acttgaggcc aggagttcga gaccagcctg agcaacatgg tgagaccctg
5581 tctctacaaa aaaaaaattt ataaaaataa aagaaaaaaa tagttatatg actaaactac
5641 tagtcaatac agcttgcaat aatagtgcat gaatgataac aggcctcggc tttatcaccc
5701 aaaaaataac ttgaattcta tgtgaaatgt gttaggataa gtttgctctg tctctagagt
5761 tcaatgtaga agataattaa gaggtaaaat atgaagctta tcatacacta aaggagaata
5821 tttggagtta gatgagcctg tcacacaaag tgaaagtgga ccctccagaa attttaggaa
5881 ttgccacaaa gattgatgat agatgaataa acagatttgt ggcctctggt aaagaaatcc
5941 agccaagttc ctctgtccaa atttttaagt gccctttgca agctaatcta tttggcaaaa
6001 aaatagtccc agttgttcaa ctagctccct taaaacattc tagtaggtgt tttaaagcct
6061 aggtatcgct gcctctttag ttcttgatga atacaaggca aaaaaaaaaa aaaaaaaaaa
6121 aaaaaaattg aggctttcac tttggcattt gcttctgttc cttgtatctg tggaagtcaa
6181 gtagcccttc tcaaaaatga gaagccaagc tttaaatagc aaactacagg gtattaaaag
6241 actaactctt cagttaagaa cactgtagaa atggtagacc aacttaaagt ctacctttta
6301 acaatcttta ccccaaacct aaagattcta ggggtcactt atctctatta cgtaatctaa
6361 ctggaataat ttcctagaat tcttactagt gaaatcttac ccaagtaaaa caatttatta
6421 ttttgaatcc aaatgaaagc tatactgttt gctttcaatg gtggcaaaaa cttgcaaaaa
6481 gcagatatac ttttgctaat ttaagttgaa gtactatgaa tacaacctgc taatcttgat
6541 ccccaaatct gagcccttca gtttcataat tgttttatcc tgaacgtagt tgtgacatag
6601 acatttctca aaagggtctg atacatcaaa ctcctaacaa gtaggagcag caactagcta
6661 aagagtagaa agaagcttcc cttattgtgg gtgatgaggc ggtgcctaaa aaccacatat
6721 atataatccc ctgggtatta catattctgc aagccctgtt agtggggaga tggtagtggc
6781 aatagcagcg atagttgcgt cagtgctaac tgctgctgct gcccgttctg ttggcggctg
6841 ctggccaagt gtgcacactt agccctggtt gaggtgcatt gtctgctgtc gtctgttaag
6901 catctaaccg gagtgggagg gcacatcaag cgatttcact ctcttaaaga aaaggttttt
6961 aggtattttt ggtattatat agtctctaag gccagaaggg gcttataatg acttctcttc
7021 tagaattgta atactaaaaa actactcgag taaatatcct aacaagtatg atttttcttg
7081 ggaaaactaa aaattgtaca tccatgcttc agccatatca tgggcctaaa ctatgagagt
7141 tcagaaatat ataaaagtac aaaaatcttc aaaggtactt atttccaaat atggagggaa
7201 tgaatctgaa gagggaacaa tggcgttttt cgaacttatt tctctctaat ccagcctgga
7261 ggatttttag ccgaaacagt tgcatgtgaa ggattatcag ccatctttgc atatcccttt
7321 cacatctacc ttcacacagg aaagctgaat gcattttacc agtgaggctc tagatatgaa
7381 acttagggat ccatagataa ataagctact aacaaactga taacagtggt gttccatctc
7441 tctatcatgt ctcctgagtc cgtttcacgt ttttgagcct ggctaatgag aaaaagaaca
7501 aagtaggacc atgacccagt gttctcctgt aaatctcact gtaaaaatct caatgcaaaa
7561 acactgagac aggagtgaga ccagctgcgg gtggggagtt tgtgctccgc aggttacctc
7621 atggcatatc aaaaatggct tttttcttca ctgcgctcaa ggaaaaagtc agatgggtga
7681 caaggtggtt ctgtttagaa gcagcaggca atgtagttaa cagcatagac ttccgtgtct
7741 tgtgtgtggt accttgggca agttactaac tccctctgta aagtagagac gatggggact
7801 gtctcactgt gacaaggtaa gatttaagtg agatgcacac aaaaagctca gcacagtgtc
7861 tggcatctaa tatgtgcagg gcagatgtca ctactgaata tcacatcaat gctagggctg
7921 tatttactgt cacacacagg caccggtaag tgactttacc ggtgaaagtc acaggtacct
7981 ttatctatga tgaatgtaat gtgttagatg gtgaccgaag tgttctccac tacacctgtc
8041 tgaagagatc taagtctgat atttaaggag tgatagcatc ttgcatactt aagaaggctt
8101 aaaagactgc atggtttaaa ccctagaaac ataaatggaa tgtttctgcc tcagttcttt
8161 tgactacctc ctaggacatg gaaacaaata tttgggagcc tgcatagtct ttagatggct
8221 gccaaagcac ttaaaatctc ctttaaattc ttaccatatg cttacttttg tactaagtaa
8281 gtgctcccaa gtcttttact tgctcttcac agcaacccta ataggtatct ccattttaca
8341 taggtaaaaa ctgaggctac aaacaatcta tggttccaaa tcctatgtgc ttactgccgt
8401 cttctcttag aaggggatgc agtactgtac aaagattgag ctggatgtcg cattgcatgt
8461 ttgctgagtc ctcctcattg ctgatgttag gatccttcaa cagtttctca gatgccagct
8521 atggctggca tccagtatgg tggtgtaatg gaagtgtact actagcacta ctacatcagg
8581 tcagcacaga ttaactcagt tacgtgttat accttcaaga cgctagacat tccagaatta
8641 ttttccttgg ccatccaagt gaagtagaag tagattgttg gattgcaaag aaaatgataa
8701 agattcccta aaggaattta taagcaggca acttcattcc tatgtataag atattgggag
8761 aaaacaaact gtaccacaac aaatcttaag tttcaaggct ctcctatagg gagaaacatg
8821 gccaaaaaga caggaagaac tgtggcttgt aactcaatga tgctataact ccagcactgg
8881 atcttaagta ttctggaata taccagattg gtagaactac cagacttggg tatttctcag
8941 ttaacacaca tgcaaggagg aagagagttt ttttgaacaa aatgactggc tttgacaaaa
9001 ctatcttgct ctttttaaat gtcacttgat actagaatgc gatttaaaag catgtaatgc
9061 ctcaggagtc cggcatatac aaccaaatta tcatgctcag gatcttcaga attctaaagg
9121 cacatgttag aatgcagagt cctgatgacc tgtcaataaa ctttttttta ctaatgaact
9181 gtacatttaa ataaaagttt atttttggga taa (SEQ ID NO: 4)

VLDLR, like LDLR, consists of 5 domains: an N-terminal 328 amino acids composed of 8 cysteine-rich repeats homologous to the ligand-binding domain of LDLR; a 396-amino acid region homologous to the epidermal growth factor precursor that mediates the acid-dependent dissociation of the ligand in the LDLR; a 46-amino acid domain homologous to the clustered O-linked sugars of the LDLR; a 22-amino acid transmembrane domain; and a 54-amino acid cytoplasmic domain including an NPXY sequence that is required for clustering of the receptor into coated pits. The gene for the VLDL receptor is highly expressed in heart, muscle, and adipose tissues, which are active in fatty acid metabolism; essentially no expression is found in liver.

The compositions and methods of the present disclosure provide gene transfer constructs that target LDLR and VLDLR, to correct pathogenic variants in the patient’s genome and to thus lower total cholesterol and/or low-density lipoprotein cholesterol (LDL-C). Accordingly, in some aspects of the present disclosure, a composition comprising a gene transfer construct is provided, comprising (a) a VLDLR or LDLR, or a functional fragment thereof, (b) a liver-specific promoter, and (c) a non-viral vector comprising one or more transposase recognition sites and one or more inverted terminal repeats (ITRs) or end sequences.

In some embodiments, the gene transfer construct comprises VLDLR, or a functional fragment thereof, instead of LDLR. VLDLR is structurally and functionally closely related to LDLR, and it predominantly modulates the extrahepatic metabolism of apoE-rich lipoproteins by their direct uptake into endothelial cells and through upregulating lipoprotein lipase-mediated metabolism of these lipoproteins. In contrast to LDLR, the expression of VLDLR is not regulated by intracellular free cholesterol. In the hepatocytes, where LDLRs are abundant, VLDLR is normally expressed only in minute amounts but can compensate for the absence of LDLRs. See Takahashi et al. J Atheroscler Thromb 2004; 11:200-8; Turunen et al. (2016). Mol Ther 2016; 24:620-35; Go & Mani. Yale J Biol Med 2012; 85:19-28.

In some embodiments, the VLDLR protein is human VLDLR protein, or a functional fragment thereof. In embodiments, the nucleic acid encoding the human VLDLR protein, or the functional fragment thereof comprises a nucleotide sequence encoding the amino acid sequence of SEQ ID NO: 6, or a variant having at least about 90%, or at least about 93%, or at least about 95%, or at least about 97%, or at least about 98% identity thereto.

In some embodiments, the nucleic acid encoding the human VLDLR, or a functional fragment thereof comprises a nucleotide sequence of SEQ ID NO: 5, or a variant having at least about 90%, or at least about 93%, or at least about 95%, or at least about 97%, or at least about 98% identity thereto:

1 atgggaactt ctgcactctg ggcattgtgg cttctgttgg ctctgtgctg ggcgccacga
61 gaaagcggag ctacaggtac tggaaggaag gccaagtgtg aacccagtca attccagtgc
121 acaaacggca ggtgtattac tctgctctgg aagtgcgacg gtgacgagga ttgtgtggac
181 ggttcagatg agaagaattg cgtcaagaag acctgcgctg aatccgattt cgtctgcaac
241 aacggccagt gtgtgccaag ccggtggaaa tgtgacggcg atcccgattg cgaagatggc
301 agcgacgagt ccccagaaca gtgccacatg aggacctgca gaattcatga gatcagctgc
361 ggagcccact ctactcagtg cattcctgtc agctggcgat gcgatggcga aaatgactgc
421 gacagcggcg aagacgaaga gaactgcggg aacatcacct gttccccgga cgaattcaca
481 tgttcctccg gacgctgtat ttccagaaac ttcgtttgca acggccagga tgactgcagc
541 gatgggtctg acgagctgga ttgcgctcct cctacatgtg gtgcacacga atttcagtgc
601 tctaccagta gctgtatacc tattagttgg gtctgtgacg atgatgccga ctgtagtgat
661 cagagtgacg agtctctgga acagtgcggc aggcaaccgg tcattcatac taaatgtccc
721 gcgagtgaga ttcagtgcgg gtcaggcgag tgcattcata agaagtggag atgcgatggc
781 gatcctgact gcaaagacgg atcagacgaa gtgaattgtc cttcccggac atgtagaccc
841 gaccagtttg aatgtgaaga tggaagctgt atacacggga gccgccagtg taacggaatc
901 agagactgcg tggatggatc tgatgaagtc aattgtaaga acgtgaatca atgccttggc
961 cccggaaaat tcaaatgcag aagtggggaa tgtattgaca tatcaaaggt ttgcaaccag
1021 gaacaagatt gccgggattg gtcagatgag ccacttaagg aatgccatat caacgaatgt
1081 ttggtgaaca acgggggctg ttcccatatc tgtaaagacc tggtaatagg ttacgagtgt
1141 gattgcgctg ccggatttga attgatcgac aggaagacct gcggtgacat tgatgagtgt
1201 cagaatccag gcatctgttc ccagatctgt attaacctca aaggaggata taaatgtgag
1261 tgctctaggg gatatcagat ggacctggca acaggagtgt gcaaggctgt tgggaaagag
1321 ccctctctga tttttaccaa cagacgagac atacgcaaga tcggactgga acggaaggag
1381 tacatccagc tcgtagagca gcttcgaaac actgtcgcgc tggacgctga catagcagcc
1441 cagaagctgt tttgggctga tctgtcccag aaggcaatct tcagcgctag catcgacgac
1501 aaagtggggc gccacgtaaa gatgattgat aacgtgtata acccagccgc gatcgcggtt
1561 gactgggtgt ataagacaat ctactggacc gatgccgcct caaaaactat ctccgtggct
1621 accttggatg gaacgaaacg caagttcttg ttcaacagtg atctcagaga acccgcatcc
1681 atagccgttg accccctctc cggctttgtg tattggtcag attggggcga gcctgcaaag
1741 atagagaagg caggcatgaa cggattcgac cggcgacctt tggttaccgc agacattcag
1801 tggccaaacg gcattacact ggacctgatc aagagcaggc tgtactggct ggactctaaa
1861 ctccacatgc tttcttccgt agacttgaat ggccaggaca ggaggatcgt gctgaaaagc
1921 ctcgagtttc tggcacaccc gctggcactg accatctttg aagatcgggt ctactggatt
1981 gatggcgaaa acgaagcggt gtacggggcc aataagttca caggctcaga gctggccacc
2041 ctggtcaata acctgaacga cgcacaggat atcatagttt atcacgagct cgtgcagcca
2101 agtgggaaga attggtgtga ggaggacatg gaaaatggcg gatgtgagta cctctgtctc
2161 ccagcaccac agatcaacga tcattcacca aagtacacgt gctcctgtcc ttccggatat
2221 aatgtggagg agaatggtag agactgccag tccacagcta ctaccgtaac gtactctgag
2281 acaaaggaca ccaacaccac cgaaatttct gctacctccg gtttggtgcc agggggcatc
2341 aacgttacca ctgccgtgtc tgaagtgtcc gtcccaccca agggaacatc agcggcatgg
2401 gctattctgc cgctgctgct gcttgttatg gcagctgttg gcggatattt gatgtggcgg
2461 aattggcagc acaaaaatat gaagtcaatg aacttcgata accctgtcta cctcaaaacc
2521 acggaggagg atctctctat tgatattggt aggcatagcg cttccgtggg tcatacttac
2581 cccgccatca gcgtggtgtc aaccgatgat gacctcgctt ga (SEQ ID NO: 5)

In some embodiments, the nucleic acid encoding human VLDLR, or a functional fragment thereof comprises an amino acid sequence of SEQ ID NO: 6, or a variant having at least about 90%, or at least about 93%, or at least about 95%, or at least about 97%, or at least about 98% identity thereto:

1 MGTSALWALW LLLALCWAPR ESGATGTGRK AKCEPSQFQC TNGRCITLLW KCDGDEDCVD
61 GSDEKNCVKK TCAESDFVCN NGQCVPSRWK CDGDPDCEDG SDESPEQCHM RTCRIHEISC
121 GAHSTQCIPV SWRCDGENDC DSGEDEENCG NITCSPDEFT CSSGRCISRN FVCNGQDDCS
181 DGSDELDCAP PTCGAHEFQC STSSCIPISW VCDDDADCSD QSDESLEQCG RQPVIHTKCP
241 ASEIQCGSGE CIHKKWRCDG DPDCKDGSDE VNCPSRTCRP DQFECEDGSC IHGSRQCNGI
301 RDCVDGSDEV NCKNVNQCLG PGKFKCRSGE CIDISKVCNQ EQDCRDWSDE PLKECHINEC
361 LVNNGGCSHI CKDLVIGYEC DCAAGFELID RKTCGDIDEC QNPGICSQIC INLKGGYKCE
421 CSRGYQMDLA TGVCKAVGKE PSLIFTNRRD IRKIGLERKE YIQLVEQLRN TVALDADIAA
481 QKLFWADLSQ KAIFSASIDD KVGRHVKMID NVYNPAAIAV DWVYKTIYWT DAASKTISVA
541 TLDGTKRKFL FNSDLREPAS IAVDPLSGFV YWSDWGEPAK IEKAGMNGFD RRPLVTADIQ
601 WPNGITLDLI KSRLYWLDSK LHMLSSVDLN GQDRRIVLKS LEFLAHPLAL TIFEDRVYWI
661 DGENEAVYGA NKFTGSELAT LVNNLNDAQD IIVYHELVQP SGKNWCEEDM ENGGCEYLCL
721 PAPQINDHSP KYTCSCPSGY NVEENGRDCQ STATTVTYSE TKDTNTTEIS ATSGLVPGGI
781 NVTTAVSEVS VPPKGTSAAW AILPLLLLVM AAVGGYLMWR NWQHKNMKSM NFDNPVYLKT
841 TEEDLSIDIG RHSASVGHTY PAISVVSTDD DLA (SEQ ID NO: 6)

In some embodiments, the VLDLR gene, or a functional fragment thereof, is mouse (Mus musculus) VLDLR. The mouse VLDLR may comprise a nucleotide sequence of SEQ ID NO: 7 encoding a protein having an amino acid sequence of SEQ ID NO: 8, or a variant having at least about 90%, or at least about 93%, or at least about 95%, or at least about 97%, or at least about 98% identity thereto.

In embodiments, the nucleic acid encoding the human VLDLR protein, or the functional fragment thereof comprises a nucleotide sequence of SEQ ID NO: 4 or a nucleotide sequence of SEQ ID NO: 5, or a variant having at least about 90%, or at least about 93%, or at least about 95%, or at least about 97%, or at least about 98% identity thereto.

In some embodiments, the nucleic acid encoding mouse VLDLR, or a functional fragment thereof comprises a nucleotide sequence of SEQ ID NO: 7, or a variant having at least about 90%, or at least about 93%, or at least about 95%, or at least about 97%, or at least about 98% identity thereto:

1 atgggtactt cagcaagatg ggcactctgg ttgctcttgg cattgtgttg ggcaccaaga
61 gatagtggag ccacagcttc cggcaaaaag gcaaagtgtg acagctcaca atttcagtgc
121 actaacggcc ggtgcataac cctcctgtgg aagtgtgatg gggacgaaga ttgcgctgat
181 ggctcagatg agaagaattg tgtgaagaag acctgtgctg agtctgattt cgtgtgtaaa
241 aacggccaat gtgtgccgaa taggtggcag tgcgacggag accccgattg cgaggatgga
301 tctgacgaga gtccggaaca gtgccatatg cgcacctgcc ggatcaacga gatctcttgt
361 ggcgctaggt ccacccaatg tatacctgta tcttggcgct gcgatgggga aaatgactgc
421 gacaatggag aggacgagga gaactgcggg aatataacat gtagcgccga tgagttcaca
481 tgctctagcg gaaggtgtgt gagccgcaat tttgtctgta acgggcaaga tgactgtgat
541 gacggatcag acgaattgga ttgtgctcca ccgacttgtg gggctcacga attccagtgt
601 agcacaagct cctgtattcc actgagctgg gtgtgcgacg acgatgctga ctgctctgat
661 cagagcgacg agtcattgga acagtgtggg cgccagccag tgattcacac taagtgcccc
721 acatctgaaa ttcaatgtgg gagtggagaa tgcattcata agaagtggag atgcgacggg
781 gacccagact gtaaagacgg gtcagatgag gtgaattgcc caagccgaac atgtcgacca
841 gaccagttcg agtgtgagga tggctcttgc atccacggga gtaggcagtg caatggaatc
901 cgggattgtg tggacggcag cgacgaagtc aactgcaaaa acgtcaatca gtgtctgggc
961 cctggaaagt tcaaatgccg gtccggagag tgtattgaca tgtccaaagt gtgcgatcag
1021 gagcaggatt gtagggattg gtccgacgaa cctctgaaag agtgtcacat caatgaatgt
1081 ctcgtaaaca acggaggctg ttcccacatt tgcaaagatc tcgtaattgg ttatgaatgc
1141 gattgtgctg ctggattcga gttgatcgat cggaagacct gcggggatat tgatgaatgt
1201 cagaatcccg gtatctgctc ccagatttgc attaatctta aaggcgggta caaatgcgag
1261 tgttcacggg ggtaccagat ggatctggct acaggggtgt gcaaggccgt tggcaaggaa
1321 cccagtttga tcttcaccaa ccgcagagat attaggaaga tcggcttgga acgcaaagag
1381 tacattcaac tggtggagca actcaggaac accgtggccc tggacgcaga tattgccgct
1441 cagaagctgt tctgggctga cttgtcccag aaggccatct tctctgccag catcgacgac
1501 aaggtaggca ggcattttaa gatgattgac aatgtgtaca acccagctgc catcgctgtg
1561 gactgggtgt acaagaccat ctattggacc gacgctgcta gcaaaacaat tagtgtggca
1621 actctggacg gagccaaacg gaagttcctc tttaactctg atttgcggga gccagcctcc
1681 atcgcagtgg atccactgtc agggttcgtt tattggtccg attggggcga gcctgctaag
1741 atagaaaagg ctggcatgaa cggttttgac cggcggccac tcgtcactga ggacatccaa
1801 tggcccaacg gtatcacact ggatctcgtg aagagccgcc tgtactggct cgatagcaag
1861 ctccacatgc tgtcctccgt ggaccttaac ggccaggacc gaaggatagt tctcaagtcc
1921 ctggagtttc tggcacatcc acttgccctt acgatatttg aagatagggt gtactggatc
1981 gatggagaaa atgaagccgt ctacggagct aacaaattta ctgggtcaga gttggctact
2041 cttgttaata acctgaacga cgcccaagac attatcgtgt accacgagct ggtgcagcca
2101 tccggcaaaa attggtgtga agatgatatg gaaaacggag gctgtgaata cctctgtctg
2161 cctgcccccc agatcaatga tcattcacca aagtatacat gcagttgccc taacggctat
2221 aacctcgagg aaaacgggag ggagtgccag agcacctcca ctccagtgac ttattcagag
2281 acaaaagata ttaatacaac ggatatattg aggacatccg gcttggttcc aggcggaatc
2341 aatgttacaa ctgcagtttc agaggttagt gtaccaccaa agggcacctc tgctgcctgg
2401 gcaatcctgc ctcttttgct ccttgttatg gccgcagtgg gaggttacct gatgtggcgc
2461 aattggcaac acaagaatat gaagtccatg aatttcgaca accctgtgta tttgaagaca
2521 acagaggagg acctgagtat tgatatcgga cgccattccg ctagtgtcgg ccatacctac
2581 ccggctataa gtgtggtgtc cactgacgac gacctggcct ga (SEQ ID NO: 7)

In some embodiments, the mouse VLDLR, or a functional fragment thereof comprises an amino acid sequence of SEQ ID NO: 8, or a variant having at least about 90%, or at least about 93%, or at least about 95%, or at least about 97%, or at least about 98% identity thereto:

1 MGTSARWALW LLLALCWAPR DSGATASGKK AKCDSSQFQC TNGRCITLLW KCDGDEDCAD
61 GSDEKNCVKK TCAESDFVCK NGQCVPNRWQ CDGDPDCEDG SDESPEQCHM RTCRINEISC
121 GARSTQCIPV SWRCDGENDC DNGEDEENCG NITCSADEFT CSSGRCVSRN FVCNGQDDCD
181 DGSDELDCAP PTCGAHEFQC STSSCIPLSW VCDDDADCSD QSDESLEQCG RQPVIHTKCP
241 TSEIQCGSGE CIHKKWRCDG DPDCKDGSDE VNCPSRTCRP DQFECEDGSC IHGSRQCNGI
301 RDCVDGSDEV NCKNVNQCLG PGKFKCRSGE CIDMSKVCDQ EQDCRDWSDE PLKECHINEC
361 LVNNGGCSHI CKDLVIGYEC DCAAGFELID RKTCGDIDEC QNPGICSQIC INLKGGYKCE
421 CSRGYQMDLA TGVCKAVGKE PSLIFTNRRD IRKIGLERKE YIQLVEQLRN TVALDADIAA
481 QKLFWADLSQ KAIFSASIDD KVGRHFKMID NVYNPAAIAV DWVYKTIYWT DAASKTISVA
541 TLDGAKRKFL FNSDLREPAS IAVDPLSGFV YWSDWGEPAK IEKAGMNGFD RRPLVTEDIQ
601 WPNGITLDLV KSRLYWLDSK LHMLSSVDLN GQDRRIVLKS LEFLAHPLAL TIFEDRVYWI
661 DGENEAVYGA NKFTGSELAT LVNNLNDAQD IIVYHELVQP SGKNWCEDDM ENGGCEYLCL
721 PAPQINDHSP KYTCSCPNGY NLEENGRECQ STSTPVTYSE TKDINTTDIL RTSGLVPGGI
781 NVTTAVSEVS VPPKGTSAAW AILPLLLLVM AAVGGYLMWR NWQHKNMKSM NFDNPVYLKT
841 TEEDLSIDIG RHSASVGHTY PAISVVSTDD DLA (SEQ ID NO: 8)

In some embodiments, the present invention relates to compositions and methods for gene transfer via a dual transposon and transposase system. Transposable elements are non-viral gene delivery vehicles found ubiquitously in nature. Transposon-based vectors have the capacity of stable genomic integration and long-lasting expression of transgene constructs in cells. Generally speaking, the dual transposon and transposase system works via a cut-and-paste mechanism whereby transposon DNA containing a transgene(s) of interest is integrated into chromosomal DNA by a transposase enzyme.

As would be appreciated in the art, a transposon often includes an open reading frame that encodes a transgene in the middle of transposon and terminal repeat sequences at 5′ and 3′ end of the transposon. The translated transposase binds to the 5′ and 3′ sequence of the transposon and carries out the transposition function.

In embodiments, a transposon is used interchangeably with transposable elements, which are used to refer to polynucleotides capable of inserting copies of themselves into other polynucleotides. The term transposon is well known to those skilled in the art and includes classes of transposons that can be distinguished on the basis of sequence organization, for example, short inverted repeats (ITRs) at each end, and/or directly repeated long terminal repeats (LTRs) at the ends. In embodiments, the transposon as described herein may be described as a piggyBac like element, e.g. a transposon element that is characterized by its traceless excision, which recognizes TTAA sequence and restores the sequence at the insert site back to the original TTAA sequence after removal of the transposon.

In some embodiments, the non-viral vector is a transposon-mediated gene transfer system (e.g., a DNA plasmid transposon system) that is flanked by ITRs recognized by a transposase. In some embodiments, the ITRs flank the VLDLR or LDLR constructs. The non-viral vector operates as a transposon-based vector system comprising a heterologous polynucleotide (also referred to as a transgene) flanked by two ends that are recognized by a transposase. The transposon ends include ITRs, which may be exact or inexact repeats and that are inverted in orientation with respect to each other. The transposase acts on the transposon ends to thereby “cut” the transposon (along with the transposon ends) from the vector and “paste,” or integrate, the transposon into a host genome.

In embodiments, a gene transfer system is a nucleic acid (DNA) encoding a transposon, and is referred to as a “donor DNA.” In embodiments, a nucleic acid encoding a transposase is helper RNA (i.e. an mRNA encoding the transposase), and a nucleic acid encoding a transposon is donor DNA (or a DNA donor transposon). In embodiments, the donor DNA is incorporated into a plasmid. In embodiments, the donor DNA is a plasmid.

DNA donor transposons, which are mobile elements that use a “cut-and-paste” mechanism, include donor DNA that is flanked by two end sequences in the case of mammals (e.g. Myotis lucifugus, Myotis myotis, Pteropus vampyrus, Pipistrellus kuhlii, and Pan troglodytes) including humans (Homo sapiens), or Inverted Terminal Repeats (ITRs) in other living organisms such as insects (e.g. Trichnoplusia ni) or amphibians (Xenopus species). Genomic DNA is excised by double strand cleavage at the hosts’ donor site and the donor DNA is integrated at this site. A dual system that uses bioengineered transposons and transposases includes (1) a source of an active transposase that “cuts” at a specific nucleotide sequences such as TTAA and (2) DNA sequence(s) that are flanked by recognition end sequences or ITRs that are mobilized by the transposase. Mobilization of the DNA sequences permits the intervening nucleic acid, or a transgene, to be inserted at the specific nucleotide sequence (i.e. TTAA) without a DNA footprint.

In embodiments, the transposase can be provided as a DNA expression vector or as an expressible RNA or a protein such that long-term expression of the transposase does not occur in the transgenic cells.

n embodiments, a transposase is a Myotis lucifugus transposase (MLT, or MLT transposase), which comprises an amino acid sequence of SEQ ID NO: 9, or a variant having at least about 90%, or at least about 93%, or at least about 95%, or at least about 97%, or at least about 98%, or at least about 99% identity thereto. In embodiments, a transposase is a Myotis lucifugus transposase (MLT, or MLT transposase), which comprises an amino acid sequence of SEQ ID NO: 9, or a variant having at least about 90%, or at least about 93%, or at least about 95%, or at least about 97%, or at least about 98%, or at least about 99% identity thereto and S2X, wherein X is any amino acid or no amino acid, optionally X is A or G. In embodiments, a transposase is a Myotis lucifugus transposase (MLT, or MLT transposase), which comprises an amino acid sequence of SEQ ID NO: 9, or a variant having at least about 90%, or at least about 93%, or at least about 95%, or at least about 97%, or at least about 98%, or at least about 99% identity thereto and S2X, wherein X is any amino acid or no amino acid, optionally X is A or G and a C terminal deletions selected from L573X and E574Xwherein X is no amino acid. In embodiments, the mutations are L573del, E574del, and S2A.

In embodiments, the MLT transposase comprises an amino acid sequence of SEQ ID NO: 9 with mutations L573del, E574del, and S2A:

MAQHSDYSDDEFCADKLSNYSCDSDLENASTSDEDSSDDEVMVRPRTLRR
RRISSSSSDSESDIEGGREEWSHVDNPPVLEDFLGHQGLNTDAVINNIED
AVKLFIGDDFFEFLVEESNRYYNQNRNNFKLSKKSLKWKDITPQEMKKFL
GLIVLMGQVRKDRRDDYWTTEPWTETPYFGKTMTRDRFRQIWKAWHFNNN
ADIVNESDRLCKVRPVLDYFVPKFINIYKPHQQLSLDEGIVPWRGRLFFR
VYNAGKIVKYGILVRLLCESDTGYICNMEIYCGEGKRLLETIQTVVSPYT
DSWYHIYMDNYVNSVANCEALMKNKFRICGTIRKNRGIPKDFQTISLKKG
ETKFIRKNDILLQVWQSKKPVYLISSIHSAEMEESQNIDRTSKKKIVKPN
ALIDYNKHMKGVDRADQYLSYYSILRRTVKWTKRLAMYMINCALFNSYAV
YKSVRQRKMGFKMFLKQTAIHWLTDDIPEDMDIVPDLQPVPSTSGMRAKP
PTSDPPCRLSMDMRKHTLQAIVGSGKKKNILRRCRVCSVHKLRSETRYMC
KFCNIPLHKGACFEKYHTLKNY (SEQ ID NO: 9),

or an amino acid sequence having at least about 90%, or at least about 93%, or at least about 95%, or at least about 97%, or at least about 98%, or at least about 99% identity thereto.

In some embodiments, an MLT transposase is encoded by the following nucleotide sequence:

atggcccagcacagcgactacagcgacgacgagttctgtgccgataagc
tgagtaactacagctgcgacagcgacctggaaaacgccagcacatccga
cgaggacagctctgacgacgaggtgatggtgcggcccagaaccctgaga
cggagaagaatcagcagctctagcagcgactctgaatccgacatcgagg
gcggccgggaagagtggagccacgtggacaaccctcctgttctggaaga
ttttctgggccatcagggcctgaacaccgacgccgtgatcaacaacatc
gaggatgccgtgaagctgttcataggagatgatttctttgagttcctgg
tcgaggaatccaaccgctattacaaccagaatagaaacaacttcaagct
gagcaagaaaagcctgaagtggaaggacatcacccctcaggagatgaaa
aagttcctgggactgatcgttctgatgggacaggtgcggaaggacagaa
gggatgattactggacaaccgaaccttggaccgagaccccttactttgg
caagaccatgaccagagacagattcagacagatctggaaagcctggcac
ttcaacaacaatgctgatatcgtgaacgagtctgatagactgtgtaaag
tgcggccagtgttggattacttcgtgcctaagttcatcaacatctataa
gcctcaccagcagctgagcctggatgaaggcatcgtgccctggcggggc
agactgttcttcagagtgtacaatgctggcaagatcgtcaaatacggca
tcctggtgcgccttctgtgcgagagcgatacaggctacatctgtaatat
ggaaatctactgcggcgagggcaaaagactgctggaaaccatccagacc
gtcgtttccccttataccgacagctggtaccacatctacatggacaact
actacaattctgtggccaactgcgaggccctgatgaagaacaagtttag
aatctgcggcacaatcagaaaaaacagaggcatccctaaggacttccag
accatctctctgaagaagggcgaaaccaagttcatcagaaagaacgaca
tcctgctccaagtgtggcagtccaagaaacccgtgtacctgatcagcag
catccatagcgccgagatggaagaaagccagaacatcgacagaacaagc
aagaagaagatcgtgaagcccaatgctctgatcgactacaacaagcaca
tgaaaggcgtggaccgggccgaccagtacctgtcttattactctatcct
gagaagaacagtgaaatggaccaagagactggccatgtacatgatcaat
tgcgccctgttcaacagctacgccgtgtacaagtccgtgcgacaaagaa
aaatgggattcaagatgttcctgaagcagacagccatccactggctgac
agacgacattcctgaggacatggacattgtgccagatctgcaacctgtg
cccagcacctctggtatgagagctaagcctcccaccagcgatcctccat
gtagactgagcatggacatgcggaagcacaccctgcaggccatcgtcgg
cagcggcaagaagaagaacatccttagacggtgcagggtgtgcagcgtg
cacaagctgcggagcgagactcggtacatgtgcaagttttgcaacattc
ccctgcacaagggagcctgcttcgagaagtaccacaccctgaagaatta
ctag (SEQ ID NO: 10),

or a nucleotide sequence having at least about 90%, or at least about 93%, or at least about 95%, or at least about 97%, or at least about 98%, or at least about 99% identity thereto.

In some embodiments, the MLT transposase (e.g., the MLT transposase having an amino acid sequence of SEQ ID NO: 9, or an amino acid sequence having at least about 90%, or at least about 93%, or at least about 95%, or at least about 97%, or at least about 98%, or at least about 99% identity thereto) comprises one or more hyperactive mutations that confer hyperactivity upon the MLT transposase. In embodiments, the hyperactive mutations, relative to the amino acid sequence of SEQ ID NO: 9 or a functional equivalent thereof, are one or more of S8X, C13X, and N125X mutations, wherein X is optionally any amino acid or no amino acid, optionally X is P, R, or K. In embodiments, the mutations are S8P, C13R, and N125K. In some embodiments, the MLT transposase has S8P and C13R mutations. In some embodiments, the MLT transposase has N125K mutation. In some embodiments, the MLT transposase has all three S8P, C13R, and N125K mutations.

In some embodiments, an MLT transposase is encoded by a nucleotide sequence (SEQ ID NO: 11) that corresponds to an amino acid (SEQ ID NO: 12) having the N125K mutation relative to the amino acid sequence of SEQ ID NO: 9 or a functional equivalent thereof, wherein SEQ ID NO: 11 and SEQ ID NO: 12 are as follows:

1 atggcccagc acagcgacta cagcgacgac gagttctgtg ccgataagct gagtaactac
61 agctgcgaca gcgacctgga aaacgccagc acatccgacg aggacagctc tgacgacgag
121 gtgatggtgc ggcccagaac cctgagacgg agaagaatca gcagctctag cagcgactct
181 gaatccgaca tcgagggcgg ccgggaagag tggagccacg tggacaaccc tcctgttctg
241 gaagattttc tgggccatca gggcctgaac accgacgccg tgatcaacaa catcgaggat
301 gccgtgaagc tgttcatagg agatgatttc tttgagttcc tggtcgagga atccaaccgc
361 tattacaacc agaag agaaa caacttcaag ctgagcaaga aaagcctgaa gtggaaggac
421 atcacccctc aggagatgaa aaagttcctg ggactgatcg ttctgatggg acaggtgcgg
481 aaggacagaa gggatgatta ctggacaacc gaaccttgga ccgagacccc ttactttggc
541 aagaccatga ccagagacag attcagacag atctggaaag cctggcactt caacaacaat
601 gctgatatcg tgaacgagtc tgatagactg tgtaaagtgc ggccagtgtt ggattacttc
661 gtgcctaagt tcatcaacat ctataagcct caccagcagc tgagcctgga tgaaggcatc
721 gtgccctggc ggggcagact gttcttcaga gtgtacaatg ctggcaagat cgtcaaatac
781 ggcatcctgg tgcgccttct gtgcgagagc gatacaggct acatctgtaa tatggaaatc
841 tactgcggcg agggcaaaag actgctggaa accatccaga ccgtcgtttc cccttatacc
901 gacagctggt accacatcta catggacaac tactacaatt ctgtggccaa ctgcgaggcc
961 ctgatgaaga acaagtttag aatctgcggc acaatcagaa aaaacagagg catccctaag
1021 gacttccaga ccatctctct gaagaagggc gaaaccaagt tcatcagaaa gaacgacatc
1081 ctgctccaag tgtggcagtc caagaaaccc gtgtacctga tcagcagcat ccatagcgcc
1141 gagatggaag aaagccagaa catcgacaga acaagcaaga agaagatcgt gaagcccaat
1201 gctctgatcg actacaacaa gcacatgaaa ggcgtggacc gggccgacca gtacctgtct
1261 tattactcta tcctgagaag aacagtgaaa tggaccaaga gactggccat gtacatgatc
1321 aattgcgccc tgttcaacag ctacgccgtg tacaagtccg tgcgacaaag aaaaatggga
1381 ttcaagatgt tcctgaagca gacagccatc cactggctga cagacgacat tcctgaggac
1441 atggacattg tgccagatct gcaacctgtg cccagcacct ctggtatgag agctaagcct
1501 cccaccagcg atcctccatg tagactgagc atggacatgc ggaagcacac cctgcaggcc
1561 atcgtcggca gcggcaagaa gaagaacatc cttagacggt gcagggtgtg cagcgtgcac
1621 aagctgcgga gcgagactcg gtacatgtgc aagttttgca acattcccct gcacaaggga
1681 gcctgcttcg agaagtacca caccctgaag aattactag (SEQ ID NO: 11),

or a nucleotide sequence having at least about 90%, or at least about 93%, or at least about 95%, or at least about 97%, or at least about 98%, or at least about 99% identity thereto (the codon corresponding to the N125K mutation is underlined and bolded).

1 MAQHSDYSDD EFCADKLSNY SCDSDLENAS TSDEDSSDDE VMVRPRTLRR PRISSSSSDS
61 ESDIEGGREE WSHVDNPPVL EDFLGHQGLN TDAVINNIED AVKLFIGDDF FEFLVEESKR
121 YYNQK RNNFK LSKKSLKWKD ITPQEMKKFL GLIVLMGQVR KDRRDDYWTT EPWTETPYFG
181 KTMTRDRFRQ IWKAWHFNNN ADIVNESDRL CKVRPVLDYF VPKFINIYKP HQQLSLDEGI
241 VPWRGRLFFR VYNAGKIVKY GILVRLLCES DTGYICNMEI YCGEGKRLLE TIQTVVSPYT
301 DSWYHIYMDN YYNSVANCEA LMKNKFRICG TIRKNRGIPK DFQTISLKKG ETKFIRKNDI
361 LLQVWQSKKP VYLISSIHSA EMEESQNIDR TSKKKIVKPN ALIDYNKHMK GVDRADQYLS
421 YYSILRRTVK WTKRLAMYMI NCALFNSYAV YKSVRQRKMG FKMFLKQTAI HWLTDDIPED
481 MDIVPDLQPV PSTSGMRAKP PTSDPPCRLS MDMRKHTLQA IVGSGKKKNI LRRCRVCSVH
541 KLRSETRYMC KFCNIPLHKG ACFEKYHTLK NY (SEQ ID NO: 12),

or an amino acid sequence having at least about 90%, or at least about 93%, or at least about 95%, or at least about 97%, or at least about 98%, or at least about 99% identity thereto (the amino acid corresponding to the N125K mutation is underlined and bolded).

In some embodiments, the MLT transposase encoded by the nucleotide sequence of SEQ ID NO: 11 and having the amino acid sequence of SEQ ID NO: 12 is referred to as an MLT transposase 1 (or MLT 1).

In some embodiments, an MLT transposase is encoded by a nucleotide sequence (SEQ ID NO: 13) that corresponds to an amino acid (SEQ ID NO: 14) having the S8P and C13R mutations relative to the amino acid sequence of SEQ ID NO: 9 or a functional equivalent thereof, wherein SEQ ID NO: 13 and SEQ ID NO: 14 are as follows:

1 atggcccagc acagcgacta cccc gacgac gagttcaga g ccgataagct gagtaactac
61 agctgcgaca gcgacctgga aaacgccagc acatccgacg aggacagctc tgacgacgag
121 gtgatggtgc ggcccagaac cctgagacgg agaagaatca gcagctctag cagcgactct
181 gaatccgaca tcgagggcgg ccgggaagag tggagccacg tggacaaccc tcctgttctg
241 gaagattttc tgggccatca gggcctgaac accgacgccg tgatcaacaa catcgaggat
301 gccgtgaagc tgttcatagg agatgatttc tttgagttcc tggtcgagga atccaaccgc
361 tattacaacc agaatagaaa caacttcaag ctgagcaaga aaagcctgaa gtggaaggac
421 atcacccctc aggagatgaa aaagttcctg ggactgatcg ttctgatggg acaggtgcgg
481 aaggacagaa gggatgatta ctggacaacc gaaccttgga ccgagacccc ttactttggc
541 aagaccatga ccagagacag attcagacag atctggaaag cctggcactt caacaacaat
601 gctgatatcg tgaacgagtc tgatagactg tgtaaagtgc ggccagtgtt ggattacttc
661 gtgcctaagt tcatcaacat ctataagcct caccagcagc tgagcctgga tgaaggcatc
721 gtgccctggc ggggcagact gttcttcaga gtgtacaatg ctggcaagat cgtcaaatac
781 ggcatcctgg tgcgccttct gtgcgagagc gatacaggct acatctgtaa tatggaaatc
841 tactgcggcg agggcaaaag actgctggaa accatccaga ccgtcgtttc cccttatacc
901 gacagctggt accacatcta catggacaac tactacaatt ctgtggccaa ctgcgaggcc
961 ctgatgaaga acaagtttag aatctgcggc acaatcagaa aaaacagagg catccctaag
1021 gacttccaga ccatctctct gaagaagggc gaaaccaagt tcatcagaaa gaacgacatc
1081 ctgctccaag tgtggcagtc caagaaaccc gtgtacctga tcagcagcat ccatagcgcc
1141 gagatggaag aaagccagaa catcgacaga acaagcaaga agaagatcgt gaagcccaat
1201 gctctgatcg actacaacaa gcacatgaaa ggcgtggacc gggccgacca gtacctgtct
1261 tattactcta tcctgagaag aacagtgaaa tggaccaaga gactggccat gtacatgatc
1321 aattgcgccc tgttcaacag ctacgccgtg tacaagtccg tgcgacaaag aaaaatggga
1381 ttcaagatgt tcctgaagca gacagccatc cactggctga cagacgacat tcctgaggac
1441 atggacattg tgccagatct gcaacctgtg cccagcacct ctggtatgag agctaagcct
1501 cccaccagcg atcctccatg tagactgagc atggacatgc ggaagcacac cctgcaggcc
1561 atcgtcggca gcggcaagaa gaagaacatc cttagacggt gcagggtgtg cagcgtgcac
1621 aagctgcgga gcgagactcg gtacatgtgc aagttttgca acattcccct gcacaaggga
1681 gcctgcttcg agaagtacca caccctgaag aattactag (SEQ ID NO: 13),

or a nucleotide sequence having at least about 90%, or at least about 93%, or at least about 95%, or at least about 97%, or at least about 98%, or at least about 99% identity thereto (the codons corresponding to the S8P and C13R mutations are underlined and bolded).

1 MAQHSDYP DD EFR ADKLSNY SCDSDLENAS TSDEDSSDDE VMVRPRTLRR RRISSSSSDS
61 ESDIEGGREE WSHVDNPPVL EDFLGHQGLN TDAVINNIED AVKLFIGDDF FEFLVEESNR
121 YYNQNRNNFK LSKKSLKWKD ITPQEMKKFL GLIVLMGQVR KDRRDDYWTT EPWTETPYFG
181 KTMTRDRFRQ IWKAWHFNNN ADIVNESDRL CKVRPVLDYF VPKFINIYKP HQQLSLDEGI
241 VPWRGRLFFR VYNAGKIVKY GILVRLLCES DTGYICNMEI YCGEGKRLLE TIQTVVSPYT
301 DSWYHIYMDN YYNSVANCEA LMKNKFRICG TIRKNRGIPK DFQTISLKKG ETKFIRKNDI
361 LLQVWQSKKP VYLISSIHSA EMEESQNIDR TSKKKIVKPN ALIDYNKHMK GVDRADQYLS
421 YYSILRRTVK WTKRLAMYMI NCALFNSYAV YKSVRQRKMG FKMFLKQTAI HWLTDDIPED
481 MDIVPDLQPV PSTSGMRAKP PTSDPPCRLS MDMRKHTLQA IVGSGKKKNI LRRCRVCSVH
541 KLRSETRYMC KFCNIPLHKG ACFEKYHTLK NY (SEQ ID NO: 14),

or an amino acid sequence having at least about 90%, or at least about 93%, or at least about 95%, or at least about 97%, or at least about 98%, or at least about 99% identity thereto (the amino acids corresponding to the S8P and C13R mutations are underlined and bolded).

In some embodiments, the MLT transposase encoded by the nucleotide sequence of SEQ ID NO: 13 and having the amino acid sequence of SEQ ID NO: 14 is referred to as an MLT transposase 2 (or MLT 2).

In some embodiments, the transposase is from a Tc1/mariner transposon system.

In some embodiments, the transposase is from a Sleeping Beauty transposon system (see, e.g. Cell. 1997;91:501-510), or a piggyBac transposon system (see, e.g. Trends Biotechnol. 2015 Sep;33(9):525-33. doi: 10.1016/j.tibtech.2015.06.009. Epub 2015 Jul 23).

In some embodiments, the transposase is from a LEAP-IN 1 type or LEAP-IN transposon system (Biotechnol J. 2018 Oct;13(10):e1700748. doi: 10.1002/biot.201700748. Epub 2018 Jun 11).

In some embodiments, a non-viral vector includes a LEAP-IN 1 type of LEAPIN Transposase (ATUM, Newark, CA). The LEAPIN Transposase system includes a transposase (e.g., a transposase mRNA) and a vector containing one or more genes of interest (transposons), selection markers, regulatory elements, etc., flanked by the transposon cognate inverted terminal repeats (ITRs) and the transposition recognition motif (TTAT). Upon co-transfection of vector DNA and transposase mRNA, the transiently expressed enzyme catalyzes high-efficiency and precise integration of a single copy of the transposon cassette (all sequences between the ITRs) at one or more sites across the genome of the host cell. Hottentot et al. In Genotyping: Methods and Protocols. White SJ, Cantsilieris S, eds: 185-196. (New York, NY: Springer): 2017. pp. 185-196. The LEAPIN Transposase generates stable transgene integrants with various advantageous characteristics, including single copy integrations at multiple genomic loci, primarily in open chromatin segments; no payload limit, so multiple independent transcriptional units may be expressed from a single construct; the integrated transgenes maintain their structural and functional integrity; and maintenance of transgene integrity ensures the desired chain ratio in every recombinant cell.

In some embodiments, the nucleic acid encoding the VLDLR or LDLR protein is operably coupled to a promoter that can influence overall expression levels and cell-specificity of the transgenes (e.g. VLDLR or LDLR, or a functional fragment thereof). In some embodiments, the promoter is tissue-specific, i.e. liver-specific promoter. In embodiments in which the transposase is a DNA sequence encoding the transposase, such DNA sequence is also operably linked to a promoter. A variety of promoters can be used, including tissue-specific promoters, inducible promoters, constitutive promoters, etc.

In some embodiments, the liver-specific promoter of the gene transfer construct can be an LP1 promoter that, in some embodiments, is a human LP1 promoter. The LP1 promoter is described, e.g., in Nathwani et al. Blood vol. 2006;107(7):2653-61.

In some embodiments, the LP1 promoter comprises a nucleic acid sequence of SEQ ID NO: 15, or a functional fragment of variant having at least about 50%, or at least about 60%, or at least about 70%, or at least about 80%, at least about 90%, or at least about 93%, or at least about 95%, or at least about 97%, or at least about 98% identity thereto. In some embodiments, the nucleotide sequence of the LP1 promoter comprises the Human apolipoprotein E/C-I gene locus, hepatic control region HCR-1 (base pairs 134 to 325, Genbank record U32510.1), Human alpha-1-antitrypsin gene (S variant) (base pairs 1747 to 2001, Genbank record K02212.1), and small t-intron SV40 (base pairs 241 to 333, Genbank record FN824656.1) (545 bp):

1 ccctaaaatg ggcaaacatt gcaagcagca aacagcaaac acacagccct ccctgcctgc
61 tgaccttgga gctggggcag aggtcagaga cctctctggg cccatgccac ctccaacatc
121 cactcgaccc cttggaattt cggtggagag gagcagaggt tgtcctggcg tggtttaggt
181 agtgtgagag gggaatgact cctttcggta agtgcagtgg aagctgtaca ctgcccaggc
241 aaagcgtccg ggcagcgtag gcgggcgact cagatcccag ccagtggact tagcccctgt
301 ttgctcctcc gataactggg gtgaccttgg ttaatattca ccagcagcct cccccgttgc
361 ccctctggat ccactgctta aatacggacg aggacagggc cctgtctcct cagcttcagg
421 caccaccact gacctgggac agtgaatccg gactctaagg taaatataaa atttttaagt
481 gtataatgtg ttaaactact gattctaatt gtttgtgtat tttagattcc aacctatgga
541 actga (SEQ ID NO: 15)

In some embodiments, the LP1 promoter comprises a nucleic acid sequence of SEQ ID NO: 15, or a variant having at least about 80%, or at least about 85%, at least about 90%, or at least about 91%, or at least about 92%, or at least about 93%, or at least about 94%, or at least about 95%, or at least about 96%, or at least about 97%, or at least about 98% identity thereto.

In some embodiments, the nucleotide sequence of the LP1 promoter comprises one or more of the Human apolipoprotein E/C-I gene locus, hepatic control region HCR-1 (base pairs 134 to 325, Genbank record U32510.1), Human alpha-1-antitrypsin gene (S variant) (base pairs 1747 to 2001, Genbank record K02212.1), and small t-intron SV40 (base pairs 241 to 333, Genbank record FN824656.1) (545 bp):

In some embodiments, the liver-specific promoter is selected from the promoters included in Table 1 below.

In some embodiments, the liver-specific promoter is selected from the promoters included in Table 1 below and optionally has one or more enhancers as listed in Table 1 and/or one or more introns as listed in Table 1.

TABLE 1

Liver-specific promoters

| | | | |
|---|---|---|---|
| Promoter Construct | Enhancer | Promoter | Intron |
| P-ALB(Hs) | none | albumin (Hs) | none |
| LP1 | ApoE/HCR1 (Hs) | antitrypsin (Hs) | SV40 small mod |
| P-C7 | | C7 Hs | none |
| P-AAT(Hs) | none | antitrypsin (Hs) | none |
| E-HBV_P-ALB(Hs) | hbv | albumin (Hs) | none |
| E-HBV_P-AAT(Hs) | hbv | antitrypsin (Hs) | none |
| E-ALB(Hs)_P-ALB(Hs) | albumin (Hs) | albumin (Hs) | none |
| E-ALB(Hs)_P-AAT(Hs) | albumin (Hs) | antitrypsin (Hs) | none |
| E-ALB(Mm)v1_P-ALB(Hs) | albumin v1 (Mm) | albumin (Hs) | none |
| E-ALB(Mm)v1_P-AAT(Hs) | albumin v1 (Mm) | antitrypsin (Hs) | none |
| E-ALB(Mm)v2_P-ALB(Hs) | albumin v2 (Mm) | albumin (Hs) | none |
| E-ALB(Mm)v2_P-AAT(Hs) | albumin v2 (Mm) | antitrypsin (Hs) | none |
| E-ALB(Hs)_P-ALB(Mm) | albumin (Hs) | albumin (Mm) | none |
| E-HBV_P-ALB(Mm) | hbv | albumin (Mm) | none |

In Table 1 above, “E” denotes enhancer, “P” denotes promoter, “Mm” denotes Mus musculus, “Hs” denotes Homo sapiens, and “HBV” denotes hepatitis B virus.

In some embodiments, the liver-specific promoter is any promoter of the promoters in Table 1 above. In some embodiments, the liver-specific promoter is any one of promoters described in Kramer et al. Mol Ther2003; 7:375-85, which is incorporated by reference herein in its entirety. In some embodiments, the promoter can be constructed as described in Kramer et al. Mol Ther 2003; 7:375-85, which is incorporated by reference herein in its entirety. In some embodiments, the liver-specific promoter is E-ALB (Mm)v2_P-AAT(Hs) or E-HBV_P-AAT (Hs), both of which are listed in Table 1 above.

In embodiments, the present non-viral vectors may comprise at least one pair of an inverted terminal repeat (ITR) at the 5′ and 3′ ends of the transposon. In embodiments, an ITR is a sequence located at one end of a vector that can form a hairpin structure when used in combination with a complementary sequence that is located at the opposing end of the vector. The pair of inverted terminal repeats is involved in the transposition activity of the transposon of the non-viral vector of the present disclosure, in particular involved in DNA addition or removal and excision of DNA of interest. In one embodiment, at least one pair of an inverted terminal repeat appears to be the minimum sequence required for transposition activity in a plasmid. In another embodiment, the vector of the present disclosure may comprise at least two, three or four pairs of inverted terminal repeats. As would be understood by the person skilled in the art, to facilitate ease of cloning, the necessary terminal sequence may be as short as possible and thus contain as little inverted repeats as possible. Thus, in one embodiment, the vector of the present disclosure may comprise not more than one, not more than two, not more than three or not more than four pairs of inverted terminal repeats. In one embodiment, the vector of the present disclosure may comprise only one inverted terminal repeat.

In embodiments, the inverted terminal repeat of the present invention may form either a perfect inverted terminal repeat (or interchangeably referred to as “perfect inverted repeat”) or imperfect inverted terminal repeat (or interchangeably referred to as “imperfect inverted repeat”). As used herein, the term “perfect inverted repeat” refers to two identical DNA sequences placed at opposite direction. In contrast, the term “imperfect inverted repeat” refers to two DNA sequences that are similar to one another except that they contain a few mismatches. These repeats (i.e. both perfect inverted repeat and imperfect inverted repeat) are the binding sites of transposase.

In some embodiments, ITRs (or end sequences) of the non-viral vector are those of a piggyBac-like transposon that transposes through a “cut-and-paste” mechanism. In some embodiments, the piggyBac-like transposon comprises a TTAA repetitive sequence. The piggyBac transposon is a frequently used transposon system for gene modifications and does not require DNA synthesis during the actual transposition event. The piggyBac element can be cut down from the donor chromosome by a transposase, and the split donor DNA can be reconnected with DNA ligase. Zhao et al. Translational lung cancer research, 2006; 5(1):120-125. The piggyBac transposon shows precise excision, i.e., restoring the sequence to its preintegration state. See Yusa. piggyBac Transposon. Microbiol Spectr. 2015 Apr;3(2). In some embodiments, the gene transfer construct comprises a Super piggyBac™ (SPB) transposase. See Barnett et al. Blood 2016; 128(22):2167.

In some embodiments, other non-viral gene transfer tools can be used such as, for example, the Sleeping Beauty transposon system. See, e.g., Aronovich et al. Human Molecular Genetics, 2011; 20(R1), R14-R20.

In some embodiments, sequences of the transposon systems can be codon optimized to provide improved mRNA stability and protein expression in mammalian systems.

In various embodiments, the gene transfer construct can be any suitable genetic construct, such as a nucleic acid construct, a plasmid, or a vector. In various embodiments, the gene transfer construct is DNA. In some embodiments, the gene transfer construct is RNA. In some embodiments, the gene transfer conduct can have DNA sequences and RNA sequences such as, e.g., miRNA (e.g. PCSK9).

In embodiments, the present nucleic acids include polymeric form of nucleotides of any length, either ribonucleotides or deoxyribonucleotides, or analogs or derivatives thereof. In embodiments, there is provided, double- and single-stranded DNA, as well as double- and single-stranded RNA, and RNA-DNA hybrids. In embodiments, transcriptionally-activated polynucleotides such as methylated or capped polynucleotides are provided. In embodiments, the present compositions are mRNA or DNA.

In embodiments, the present non-viral vectors are linear or circular DNA molecules that comprise a polynucleotide encoding a polypeptide and is operably linked to control sequences, wherein the control sequences provide for expression of the polynucleotide encoding the polypeptide. In embodiments, the non-viral vector comprises a promoter sequence, and transcriptional and translational stop signal sequences. Such vectors may include, among others, chromosomal and episomal vectors, e.g., vectors derived from bacterial plasmids, from transposons, from yeast episomes, from insertion elements, from yeast chromosomal elements, and vectors derived from combinations thereof. The present constructs may contain control regions that regulate as well as engender expression.

In some embodiments, the gene transfer construct can be codon-optimized. In the described embodiments, the nucleic acid encoding the VLDLR protein and LDLR protein, or a functional fragment thereof, function as transgenes that are integrated into a host genome (e.g., a human genome) to provide desired clinical outcomes. Transgene codon optimization can be used to optimize therapeutic potential of the transgene and its expression in the host organism. Codon optimization is performed to match the codon usage in the transgene with the abundance of transfer RNA (tRNA) for each codon in a host organism or cell. Codon optimization methods are known in the art and described in, for example, WO 2007/142954, which is incorporated by reference herein in its entirety. Optimization strategies can include, for example, the modification of translation initiation regions, alteration of mRNA structural elements, and the use of different codon biases.

The gene transfer construct includes several other regulatory elements that are selected to ensure stable expression of the construct. Thus, in some embodiments, the non-viral vector is a DNA plasmid that can comprise one or more insulator sequences that prevent or mitigate activation or inactivation of nearby genes.

In embodiments, the ITRs or the end sequences are those of a piggyBac-like transposon, optionally comprising a TTAA repetitive sequence, and/or the ITRs or the end sequences flank the nucleic acid encoding the VLDLR protein or LDLR protein, or a functional fragment thereof.

In some embodiments, the one or more insulator sequences comprise an HS4 insulator (1.2-kb 5′-HS4 chicken β-globin (cHS4) insulator element) and an D4Z4 insulator [tandem macrosatellite repeats linked to Facio-Scapulo-Humeral Dystrophy (FSHD)]. In some embodiments, the sequences of the HS4 insulator and the D4Z4 insulator are as described in Rival-Gervier et al. Mol Ther. 2013 Aug; 21(8):1536-50, which is incorporated herein by reference in its entirety. In some embodiments, the gene of the gene transfer construct is capable of transposition in the presence of a transposase. In some embodiments, the non-viral vector in accordance with embodiments of the present disclosure comprises a nucleic acid construct encoding a transposase. The transposase can be a transposase DNA plasmid. In some embodiments, the transposase is in vitro-transcribed mRNA. The transposase is capable of excising and/or transposing the gene from the gene transfer construct to site- or locus-specific genomic regions.

A composition comprising a gene transfer construct in accordance with the present disclosure can include one or more non-viral vectors. Also, the transposase can be disposed on the same or different vector than a transposon with a transgene. Accordingly, in some embodiments, the transposase and the transposon encompassing a transgene are in cis configuration such that they are included in the same vector. In some embodiments, the transposase and the transposon encompassing a transgene are in trans configuration such that they are included in different vectors. The vector is any non-viral vector in accordance with the present disclosure.

In some embodiments, the transposase is derived from Bombyx mori, Xenopus tropicalis, Trichoplusia ni, Rhinolophus ferrumequinum, Rousettus aegyptiacus, Phyllostomus discolor, Myotis myotis, Myotis lucifugus, Pteropus vampyrus, Pipistrellus kuhlii, Pan troglodytes, Molossus molossus, or Homo sapiens, and/or is an engineered version thereof. In some embodiments, the transposase specifically recognizes the ITRs. The transposase can include DNA or RNA sequences encoding Bombyx mori, Xenopus tropicalis, or Trichoplusia ni proteins. See, e.g., U.S. Pat. No. 10,041,077, which is incorporated herein by reference in its entirety.

In some embodiments, however, a transposase may be introduced into the cell directly as protein, for example using cell-penetrating peptides (e.g., as described in Ramsey and Flynn (2015). Cell-penetrating peptides transport therapeutics into cells. Pharmacol. Ther. 154: 78-86); using small molecules including salt plus propanebetaine (e.g., as described in Astolfo et al. Cell 2015; 161:674-690); or electroporation (e.g., as described in Morgan and Day. Methods in Molecular Biology 1995; 48: 63-71).

In some embodiments, the transposon system can be implemented as described, e.g., in U.S. Pat. No. 10,435,696, which is incorporated herein by reference in its entirety.

In some embodiments, the described composition includes a transgene (e.g., a nucleic acid encoding VLDLR protein or LDLR protein, or a functional fragment thereof) and a transposase in a certain ratio. In some embodiments, a transgene to transposase ratio is selected that improves efficiency of transpositional activity. The transgene to transposase ratio is dependent on the concentration of the transfected gene transfer construct. In some embodiments, the ratio of the nucleic acid encoding the very low-density lipoprotein receptor protein (VLDLR) or the low-density lipoprotein receptor protein (LDLR), or a functional fragment thereof to the nucleic acid construct encoding transposase is about 5:1, or about 4:1, or about 3:1, or about 2:1, or about 1:1, or about 1:2, or about 1:3, or about 1:4, or about 1:5.

In some embodiments, the ratio of the nucleic acid encoding the very low-density lipoprotein receptor protein (VLDLR) or low-density lipoprotein receptor protein (LDLR), or a functional fragment thereof to the nucleic acid construct encoding transposase is about 2:1.

In some embodiments, the non-viral vector is a conjugated polynucleotide sequence that is introduced into cells by various transfection methods such as, e.g., methods that employ lipid particles. In some embodiments, a composition, including a gene transfer construct, comprises a delivery particle. In some embodiments, the delivery particle comprises a lipid-based particle (e.g., a lipid nanoparticle (LNP)), cationic lipid, or a biodegradable polymer). Lipid nanoparticle (LNP) delivery of gene transfer construct provides certain advantages, including transient, non-integrating expression to limit potential off-target events and immune responses, and efficient delivery with the capacity to transport large cargos. LNPs have been used for delivery of small interfering RNA (siRNA) and mRNA, and for in vitro and in vivo delivering CRISPR/Cas9 components to hepatocytes and the liver. For example, U.S. Pat. No. 10,195,291 describes the use of LNPs for delivery of RNA interference (RNAi) therapeutic agents.

In some embodiments, the composition in accordance with embodiments of the present disclosure is in the form of a LNP. In some embodiments, the LNP comprises one or more lipids selected from 1,2-dioleoyl-3-trimethylammonium propane (DOTAP); N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB), a cationic cholesterol derivative mixed with dimethylaminoethane-carbamoyl (DC-Chol), phosphatidylcholine (PC), triolein (glyceryl trioleate), and 1,2-distearoyl-sn-glycero-3-phosphoethanolamine-N-[carboxy(polyethylene glycol)-2000] (DSPE-PEG), 1,2-dimyristoyl-rac-glycero-3-methoxypolyethyleneglycol -2000 (DMG-PEG 2 K), and 1,2 distearol-sn-glycerol-3phosphocholine (DSPC).

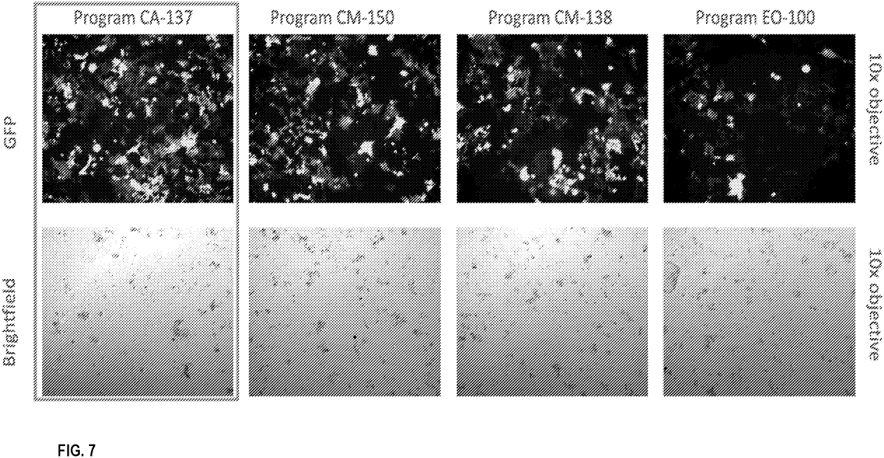

In some embodiments, an LNP can be as shown in FIG. 5, which is adapted from Patel et al., J Control Release 2019; 303:91-100. As shown in FIG. 5, the LNP can comprise one or more of a structural lipid (e.g., DSPC), a PEG-conjugated lipid (CDM-PEG), a cationic lipid (MC3), cholesterol, and a targeting ligand (e.g. GalNAc).

In some embodiments, the composition can have a lipid and a polymer in various ratios, wherein the lipid can be selected from, e.g., DOTAP, DC-Chol, PC, Triolein, DSPE-PEG, and wherein the polymer can be, e.g., PEI or Poly Lactic-co-Glycolic Acid (PLGA). Any other lipid and polymer can be used additionally or alternatively. In some embodiments, the ratio of the lipid and the polymer is about 0.5:1, or about 1:1, or about 1:1.5, or about 1:2, or about 1:2.5, or about 1:3, or about 3:1, or about 2.5:1, or about 2:1, or about 1.5:1, or about 1:1, or about 1:0.5.

In some embodiments, the LNP comprises a cationic lipid, non-limiting examples of which include N,N-dioleyl-N,N-dimethylammonium chloride (DODAC), N,N-distearyl-N,N-dimethylammonium bromide (DDAB), N-(I-(2,3-dioleoyloxy)propyl)-N,N,N-trimethylammonium chloride (DOTAP), N-(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.CI), 1,2-Dilinoleoyl-3-trimethylaminopropane chloride salt (DLin-TAP.CI), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5 s,6aS)-N,N-dimethyl-2,2-di((9Z,12Z)-octadeca-9,12-dienyl)tetrahydro-3aH-cyclopenta[d][1,3]dioxol-5-amine (ALN100), (6Z,9Z,28Z,31Z)-heptatriaconta-6,9,28,31-tetraen-19-yl 4-(dimethylamino)butanoate (MC3), 1,1′-(2-(4-(2-((2-(bis(2-’)amino)ethyl)(2 hydroxydodecyl)amino)ethyl)piperazin-1-yl)ethylazanediyl)didodecan-2-ol (Tech G1), 1,2-Dilinoleyloxo-3-(2-N,N-dimethylamino)ethoxypropane (DLin-EG-DMA), or a mixture thereof.

In some embodiments, the LNP comprises one or more molecules selected from polyethylenimine (PEI) and poly(lactic-co-glycolic acid) (PLGA), and N-Acetylgalactosamine (GalNAc), which are suitable for hepatic delivery. In some embodiments, the LNP comprises a hepatic-directed compound as described, e.g., in U.S. Pat. No. 5,985,826, which is incorporated by reference herein in its entirety. GalNAc is known to target Asialoglycoprotein Receptor (ASGPR) expressed on mammalian hepatic cells. See Hu et al. Protein Pept Lett. 2014;21(10):1025-30.

In some examples, the gene transfer constructs of the present disclosure can be formulated or complexed with PEI or a derivative thereof, such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives.

In some embodiments, the LNP is a conjugated lipid, non-limiting examples of which include a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate may be, for example, a PEG-dilauryloxypropyl (C12, a PEG-dimyristyloxypropyl (C14), a PEG-dipalmityloxypropyl (C16), or a PEG-distearyloxypropyl (C18).

In embodiments, a nanoparticle is a particle having a diameter of less than about 1000 nm. In some embodiments, nanoparticles of the present disclosure have a greatest dimension (e.g., diameter) of about 500 nm or less, or about 400 nm or less, or about 300 nm or less, or about 200 nm or less, or about 100 nm or less. In some embodiments, nanoparticles of the present invention have a greatest dimension ranging between about 50 nm and about 150 nm, or between about 70 nm and about 130 nm, or between about 80 nm and about 120 nm, or between about 90 nm and about 110 nm. In some embodiments, the nanoparticles of the present invention have a greatest dimension (e.g., a diameter) of about 100 nm.

In some aspects, the compositions in accordance with the present disclosure can be delivered via an in vivo genetic modification method. In some embodiments, a genetic modification in accordance with the present disclosure can be performed via an ex vivo method.

Accordingly, in some embodiments, a method for lowering total cholesterol and/or low-density lipoprotein cholesterol (LDL-C) in a patient is provided that comprises administering to a patient in need thereof a composition according to any embodiment, or a combination of embodiments, of the present disclosure. The method includes delivering the composition via a suitable route, including intravenous or intraperitoneal administration, or administration to the liver, optionally to the intraportal vein or liver parenchyma.

In some aspects, the present invention provides an ex vivo gene therapy approach. Accordingly, in some aspects, a method for lowering total cholesterol and/or LDL-C in a patient is provided that comprises (a) contacting a cell obtained from a patient with a composition in accordance with embodiments of the present disclosure; and (b) administering the cell to a patient in need thereof.

In embodiments, the method further comprises contacting the cells with:a nucleic acid construct encoding a transposase, optionally derived from Bombyx mori, Xenopus tropicalis, Trichoplusia ni, Rhinolophus ferrumequinum, Rousettus aegyptiacus, Phyllostomus discolor, Myotis myotis, Myotis lucifugus, Pteropus vampyrus, Pipistrellus kuhlii, Pan troglodytes, Molossus molossus, or Homo sapiens, and/or an engineered version thereof, and/or with a nucleic acid construct encoding a microRNA that targets PCSK9.

In some embodiments, the in vivo and ex vivo methods described herein can treat familial hypercholesterolemia, common hypercholesterolemia, increased triglycerides, insulin resistance, metabolic syndrome and other diseases characterized by elevated levels of cholesterol.

In some aspects, an ex vivo method for treating and/or mitigating FH is provided that comprises (a) contacting a cell obtained from a patient with a composition in accordance with embodiments of the present disclosure, and (b) administering the cell to a patient in need thereof. In some embodiments, the FH is homozygous FH (HoFH). In some embodiments, the FH is heterozygous FH (HeFH). In some embodiments, the FH is characterized by one or more mutations in one or more of APOB, LDLR, and PCSK9. These pathogenic mutations can be corrected using the described methods for treating and/or mitigating high LDL-C levels in the serum.

In some aspects, the method for treating and/or mitigating FH is provided that comprises administering to a patient in need thereof a composition in accordance with embodiments of the present disclosure. In such in vivo method, the composition is administered using any of the techniques described herein.

In embodiments, the method for treating and/or mitigating FH further comprises administering a miRNA that targets PCSK9.

One of the advantages of ex vivo gene therapy is the ability to “sample” the transduced cells before patient administration. This facilitates efficacy and allows performing safety checks before introducing the cell(s) to the patient. For example, the transduction efficiency and/or the clonality of integration can be assessed before infusion of the product. The liver has a unique regenerative capacity, with both parenchymal and nonparenchymal cells contributing to this process. Upon liver injury, hepatic cells can change into partially dedifferentiated progenitors, which yield hepatocytes and bile duct epithelial cells that can restore the organ’s original size and normal function.

Nevertheless, primary human hepatocytes (PHHs) do not spontaneously divide in vitro. This is an important limitation for ex vivo disease modeling and cell-based therapy directed toward the liver. Even though ex vivo transplantation of genetically modified cells is an attractive alternative to liver transplantation, attempts to employ this technology has been unsuccessful so far. Surgical hepatic transplantation remains to be curative/palliative for a number of inherited and acquired hepatic diseases but is hampered by the shortage of donors. See, e.g., McDiarmid, Liver Transpl 2001; 7:48-50.

In some embodiments, the isolated cell is a hepatocyte, which can be a modified hepatocyte. In some embodiments, the isolated cell is a primary human hepatocyte (PHH). In some embodiments, the isolated cell is an induced pluripotent stem cell.

Induced pluripotent stem cells (iPSCs) have the potential to serve as a surrogate to stem cell transplantation. Reprogramming of adult cells into iPSCs directs the trans-differentiation of fibroblasts to hepatocytes, circumventing the pluripotent state. See, e.g., Yu et al. (2013). Cell Stem Cell 13:328-40; Takahashi & Yamanaka (2006). Cell 126:663-76; Zhu et al. (2014) Nature 508:93-7. iPSCs are endowed with intrinsic self-renewal ability and the potential to differentiate into any of the three germ layers, allowing them to produce large amounts of gene-corrected transplantable hepatocytes for the treatment of congenital liver diseases. However, the generation of iPSCs for transplantation is limited by the occurrence of epigenetic abnormalities and chromosomal rearrangements, as well as formation of teratomas. See, e.g., Liang & Zhang (2013). Cell Stem Cell 13:149-59; Si-Tayeb et al. (2010) Hepatology 51:297-305; Ma et al. (2014) Nature 511:177-83.

Hepatocytes have generally not been considered good candidates for ex vivo type of genetic manipulation due to their quiescence in the absence of liver damage, but recent studies have demonstrated that their proliferation can be induced in vitro, albeit not to the extent needed for human gene therapy. Levy et al. Nat Biotechnol 2015; 33:1264-71. Recently however, Unzu et al. developed a method for generating proliferative human hepatic progenitor cells (HPCs) by ex vivo exposure of human primary liver cells to a cocktail of growth factors and small molecules mimicking Wnt, EGF, and FGF signaling, which resulted in efficient reprogramming of PHHs into precursor cells. Unzu et al. Hepatology 2019;69:2214-31. The present disclosure provides compositions and methods that can be effectively used for ex vivo gene modification.

In some embodiments, any of the in vivo and ex vivo methods described herein improve cardiovascular health of the patient. In some embodiments, the method is substantially non-immunogenic.

In some embodiments, the method requires a single administration. Accordingly, the present invention may provide a one-time-only therapeutic, which can significantly improve the current treatment protocols that are often life-long and involve multiple medications and/or procedures (e.g., LDL apheresis).

In some embodiments, the method stimulates and/or increases LDL metabolism in hepatocytes. In some embodiments, the lowering of total cholesterol and/or LDL-C is durable.

In some embodiments, the method allows treating and/or mitigating coronary artery disease (CAD). In some embodiments, the method allows treating and/or mitigating atherosclerosis.

In some embodiments, the method provides greater than about a 40%, or greater than about a 50%, or greater than about a 60%, or greater than about a 70%, or greater than about a 80%, or greater than about a 90% lowering of total cholesterol and/or LDL-C relative to a level of total cholesterol and/or LDL-C without the administration.

In some embodiments, the method lowers LDL-C levels to less than about 500 mg/dL (less than about 13 mmol/L) in the serum. In some embodiments, the method lowers serum LDL-C levels to less than about 450 mg/dL, or less than about 400 mg/dL, or less than about 350 mg/dL, or less than about 300 mg/dL, or less than about 250 mg/dL, or less than about 200 mg/dL, or less than about 150 mg/dL. In some embodiments, the method lowers serum LDL-C levels to less than about 130 mg/dL.

In some embodiments, the present compositions and methods allow lowering PCSK9, which can be done by using PCSK9-targeting microRNA (miRNA).

In embodiments, the present gene-targeting compositions can knockdown PCSK9. In embodiments, the present gene-targeting compositions can prevent LDLR and VLDLR degradation and turnover.

In embodiments, the present gene-targeting compositions comprise a microRNA that targets PCSK9. In embodiments, the microRNA sequence that targets PCSK9 is under the control of a different promoter than the LDLR or VLDLR genes. In embodiments, the microRNA sequence that targets PCSK9 is under the control of a CAG promoter (see, e.g. Gene. 79 (2): 269-77). In embodiments, the microRNA sequence that targets PCSK9 is in the context of a miR-451 scaffold, which, without wishing to be bound by theory, can force miRNA processing via the non-canonical Dicer-independent pathways and avoid the production of off-target effects due to passenger strand activity. Herrera-Carrillo et al., Nucleic Acids Res 2017;45:10369-79.

In embodiments, the present gene-targeting compositions find use in a method involving an additional composition comprising a gene-targeting composition comprising a microRNA that targets PCSK9. In embodiments, the microRNA sequence that targets PCSK9 is under the control of a different promoter than the LDLR or VLDLR genes. In embodiments, the microRNA sequence that targets PCSK9 is under the control of a CAG promoter (see, e.g. Gene. 79 (2): 269-77). In embodiments, the microRNA sequence that targets PCSK9 is in the context of a miR-451 scaffold.

In embodiments, the microRNA that targets PCSK9 is one of miR-24, miR-191, miR-195, miR-222, and miR-224.

PCSK9 promotes the degradation of LDL receptors by forming a complex with the receptors, mainly in the liver. Localized on the cell surface, LDL receptors bind with LDL, and afterwards, the complex is transported to the endosomes via endocytosis and release the LDL under acidic conditions. LDL is degraded to amino acids and cholesterol, while LDL receptors are transported to the cell surface, bind with LDL and taken into cells. This recycling of LDL receptors to the cell surface occurs approximately 150 times. PCSK9 is secreted by the endoplasmic reticulum in liver cells, binds with LDL receptors on the cell membrane, and is taken into cells. LDL receptors that PCSK9 has bound to are degraded in lysosomes without being recycled. PCSK9 also regulates the LDLR paralog, VLDLR, and limits adipogenesis via regulation of adipose VLDLR levels.

Because PCSK9 promotes the degradation of LDL receptors, targeting PCSK9 can affect LDL cholesterol levels in plasma. Loss of function mutations in humans are associated with low LDL-C levels and lower rates of cardiovascular disease. See Cohen et al., N Engl J Med 2006;354: 1264-72; Miyake et al. Atherosclerosis 2008;196:29-36. A complete loss of PCSK9 function is not considered to influence viability or health in humans or mammals. See Zhao et al., Am J Hum Genet 2006;79:514-23; Rashid et al., Proc Natl Acad Sci USA 2005;102:5374-9. The loss of PCSK9 function leads to lowering of LDL-C and, in a retrospective outcome study of over 15 years, loss of one copy of PCSK9 was shown to shift LDL-C lower and lead to an increased risk:benefit protection from developing cardiovascular disease. Cohen et al., N Engl J Med 2006;354:1264-72.

RNA interference and related RNA silencing pathways harness a highly specific endogenous mechanism for regulating gene expression. Small interfering RNAs (siRNAs) selectively and catalytically silence the translation of their complementary target messenger RNAs (mRNAs) in a sequence-specific manner through the formation of effector RNA-induced silencing complexes. Recently, inclisiran, an siRNA, was shown to lower PCSK9 and LDL cholesterol levels among patients at high cardiovascular risk who had elevated LDL cholesterol levels. Ray et al., N Engl J Med 2017;376:1430-40. PCSK9 silencing using PCSK9 sequence-specific miRNA constructs can be designed with high gene silencing activity. An approach that uses a miRNA miR-451 scaffold can force miRNA processing via the non-canonical Dicer-independent pathways and avoid the production of off-target effects due to passenger strand activity. Herrera-Carrillo et al., Nucleic Acids Res 2017;45:10369-79.

In some embodiments, a nucleic acid construct encoding a transposase is delivered, optionally derived from Bombyx mori, Xenopus tropicalis, or Trichoplusia ni and/or an engineered version thereof.

In some embodiments, the cells are contacted with a nucleic acid construct encoding a transposase, optionally derived from Bombyx mori, Xenopus tropicalis, or Trichoplusia ni and/or an engineered version thereof.

In some embodiments, a transposase is derived from Bombyx mori, Xenopus tropicalis, Trichoplusia ni, Rhinolophus ferrumequinum, Rousettus aegyptiacus, Phyllostomus discolor, Myotis myotis, Myotis lucifugus, Pteropus vampyrus, Pipistrellus kuhlii, Pan troglodytes, Molossus molossus, or Homo sapiens, and/or an engineered version thereof.

In some embodiments, the method of treating and/or mitigating FH is performed in the absence of a steroid treatment. Steroids, such as glucocorticoid steroids (e.g., prednisone) have been used to improve effectiveness of AAV-based gene therapy by reducing immune response. However, steroid treatment is not without side effects. The compositions and methods of the present disclosure can be substantially non-immunogenic, and can therefore eliminate the need for a steroid treatment.

In some embodiments, however, the method is performed in combination with a steroid treatment.

In some embodiments, the method can be used to administer the described composition in combination with one or more additional therapeutic agents. Non-limiting examples of the additional therapeutic agents comprise one or more of a statin, ezetimibe, a bile-acid binding resin, evolocumab, inclisiran, lomitapide and mipomersen. Accordingly, in some embodiments, the method further comprises administering one or more of a statin, ezetimibe, a bile-acid binding resin, evolocumab, inclisiran, lomitapide and mipomersen. In some embodiments, statin is one or more of Atorvastatin (LIPITOR), fluvastatin (LESCOL), lovastatin (ALTOCOR; ALTOPREV; MEVACOR), pitavastatin (LIVALO), Pravastatin (PRAVACHOL), rosuvastatin calcium (CRESTOR), and simvastatin (ZOCOR).

In aspects, a composition comprising a gene transfer construct is provided. In embodiments, the composition comprises (a) a the nucleic acid encoding a very low-density lipoprotein receptor protein (VLDLR) or a low-density lipoprotein receptor protein (LDLR) or a functional fragment thereof, wherein the VLDLR is human VLDLR that comprises a nucleotide sequence of SEQ ID NO: 4, or a variant of about 90% identity thereto, or a nucleotide sequence of SEQ ID NO: 5, or a variant of about 90% identity thereto;(b) a liver-specific promoter, wherein the liver-specific promoter is a human LP1 promoter having a nucleic acid sequence of SEQ ID NO: 15, or a variant of having least about 90% identity thereto; and (c) a non-viral vector comprising one or more transposase recognition sites and one or more inverted terminal repeats (ITRs) or end sequences.

In some embodiments, the method obviates the need for treatment with one or more of a statin, ezetimibe, a bile-acid binding resin, evolocumab, inclisiran, lomitapide and mipomersen. The present methods and compositions can provide durable decrease in the levels of total cholesterol and/or LDL-C, and the need for additional therapeutic agents can therefore be decreased or eliminated.

Furthermore, in some embodiments, the method obviates the need for LDL apheresis.

In some embodiments, the compositions for non-viral gene therapy in accordance with the present disclosure are administered via various delivery routes, including intravenous, intraportal, hydrodynamic delivery, and direct injection. In some embodiments, the administering is intravenous. In some embodiments, the administration is intraportal or direct injection into liver parenchyma. For example, in an embodiment, the administration is intraparenchymal liver injection uses convection-enhanced delivery (CED). CED is a technique that generates a pressure gradient at a tip of a micro-step infusion cannula to deliver a therapeutic agent directly through the interstitial spaces of the central nervous system. Mehta et al. (2017). Neurotherapeutics: the Journal of the American Society for Experimental NeuroTherapeutics, 14(2), 358-371.

In some embodiments, the administering of the cell to a patient is intravenous. The intravenous administration is a common approach, which in some cases however may require an increased total dose and may lead to transduction of non-target organs. In some cases, also, the intravenous administration may increase the chances on transducing germ cells in the ovaries and testes. This is undesirable in an integrating gene therapy using transposases.

In some embodiments, the administering to the cell is intra-arterial, intraportal, and or retrograde intravenous routes.

In some embodiments, the administering of the cell to a patient is intraportal.

In some embodiments, the administering of the cell to a patient is a direct intraparenchymal hepatic administration. In humans, percutaneous liver biopsy is a procedure in which a long needle is introduced through the skin, subcutaneous tissues, intercostal muscles, and peritoneum into the liver to obtain a specimen of liver tissue. This procedure is usually performed on an outpatient basis. In some embodiments, a direct injection can follow a similar procedure, and a microcannulae can be used to inject the composition by convection-enhanced delivery (CED). CED is a direct infusion technique that relies on pressure-driven bulk flow. It is currently used to deliver gene therapy to the central nervous system (CNS). The bulk flow is created by a small pressure gradient from a pump that pushes solute through a catheter targeted within the CNS that provides much greater volumes of drug distribution than are achievable through diffusion. Compared with traditional convection delivery, CED produces higher concentrations of therapeutic agents, longer infusion times, and larger distribution volumes at the infusion site, resulting in minimal tissue injury. The infusion rates in the CNS range from 3 to 15 microliters/min. In some embodiments, the infusion rates can be higher than 15 microliters/min.

In some embodiments, a liver biopsy is performed with an imaging guidance. In some embodiments, the liver biopsy is guided using an imaging technique, including ultrasound guidance, transjugular, computerized tomography (CT), Magnetic Resonance Imaging (MRI), laparoscopic, and endoscopic ultrasound. A percutaneous CED infusion of the right lobe of the liver (⅚ of the liver volume) may be used in some embodiments.

In some embodiments, the composition in accordance with the present disclosure comprises a pharmaceutically acceptable carrier, excipient or diluent.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Therapeutic compounds can be prepared with carriers that will protect the therapeutic compounds against rapid elimination from the body, such as a controlled release formulation, including implants and microencapsulated delivery systems. Biodegradable, biocompatible polymers can be used, such as collagen, ethylene vinyl acetate, polyanhydrides (e.g., poly[1,3-bis(carboxyphenoxy)propane-co-sebacic-acid] (PCPP-SA) matrix, fatty acid dimer-sebacic acid (FAD-SA) copolymer, poly(lactide-co-glycolide)), polyglycolic acid, collagen, polyorthoesters, polyethyleneglycol-coated liposomes, and polylactic acid. Such formulations can be prepared using standard techniques, or obtained commercially, e.g., from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811. Semisolid, gelling, soft-gel, or other formulations (including controlled release) can be used, e.g., when administration to a surgical site is desired. Methods of making such formulations are known in the art and can include the use of biodegradable, biocompatible polymers. See, e.g., Sawyer et al., Yale J Biol Med. 2006; 79(3-4): 141-152.

In embodiments, there is provided a method of transforming a cell using the gene transfer constructs described herein in the presence of a transposase to produce a stably transfected cell which results from the stable integration of a gene of interest into the cell. In embodiments, the stable integration comprises an introduction of a polynucleotide into a chromosome or mini-chromosome of the cell and, therefore, becomes a relatively permanent part of the cellular genome.

In embodiments, the present invention relates to determining whether a gene of interest, e.g. VLDLR or LDLR, has been successfully transferred into a genome of a host. In one embodiment, the method may include performing a polymerase chain reaction with primers flanking the gene of interest; determining the size of the amplified polymerase chain reaction products obtained; and comparing the size of products obtained with a reference size, wherein if the size of the products obtained matches the expected size, then the gene of interest was successfully transferred.

In embodiments, there is provided a host cell comprising a composition as described herein (e.g., without limitation, a composition comprising the gene transfer construct and/or transposase). In embodiments, the host cell is a prokaryotic or eukaryotic cell, e.g. a mammalian cell.

In embodiments, there is provided a transgenic organism that may comprise cells which have been transformed by the methods of the present disclosure. In one example, the organism may be a mammal or an insect. When the organism is a mammal, the organism may include, but is not limited to, a mouse, a rat, a monkey, a dog, a rabbit and the like. When the organism is an insect, the organism may include, but is not limited to, a fruit fly, a mosquito, a bollworm and the like.

The compositions can be included in a container, kit, pack, or dispenser together with instructions for administration.

Also provided herein are kits comprising: i) any of the aforementioned gene transfer constructs of this invention, and/or any of the aforementioned cells of this invention and ii) a container. In certain embodiments, the kits further comprise instructions for the use thereof. In certain embodiments, any of the aforementioned kits can further comprise a recombinant DNA construct comprising a nucleic acid sequence that encodes a transposase.

This invention is further illustrated by the following non-limiting examples.

### EXAMPLES

### Example 1: Design of a Transposon Expression Vector

A non-viral, transposon expression vector is designed and cloned using the LEAPIN Transposase technology (ATUM, Newark, CA). The transposon expression vector includes human VLDLR operably linked to and driven by the LP1 promoter. A transposon expression vector including mouse LP1-vldlr is also created. A Human LDRL-/-individual pluripotent stem cell (iPSC) disease cell line or transformed human or embryonic stem cells (ESC) are used. Other tested cell lines include human liver (HepG2, Huh7), and non-liver (CHO, HT1080, and HEK293) cell lines. The B6.129S7-Ldlrtm1Her/J (JAX) mouse model (ldlr -/-) is used.

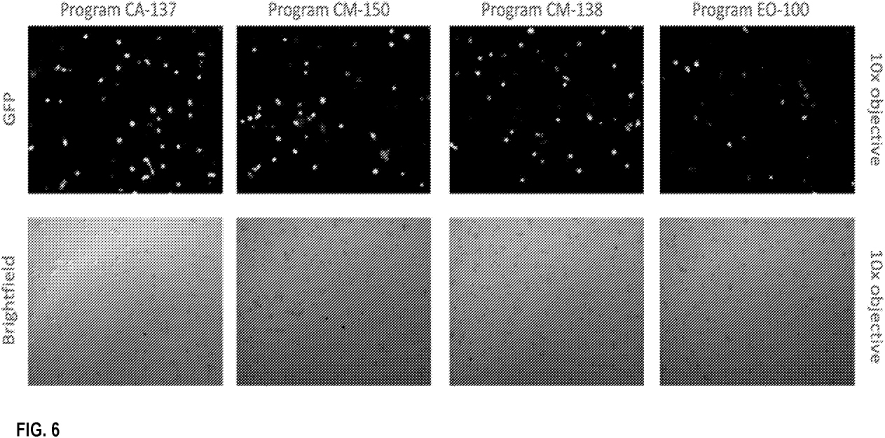

FIGS. 4A, 4B, 4C, 4D, 4E, 4F, 4G, and 4H show schematic representations of the constructs used in vitro transfection and assessment of transposition efficacy, and expression studies in human stem cells and LP1-luciferase biodistribution in ldlr -/- mice. FIG. 4A shows a transposon construct with phosphoglycerate kinase (PGK) promoter. FIGS. 4B and 4C show a LP1-GFP transposon construct that is used to determine the transposition efficacy of the designed expression vector. FIG. 4D shows a LP1-VLDRL construct that is used to assess the phenotypic consequences of transfection of stem cells with VLDLR after differentiation to hepatocytes. The LP1-VLDRL constructs are prepared that include mice, pigs, cynomolgus monkeys, and human sequences. FIG. 4E shows an LP1-enhanced human VLDLR (e-LDLR) construct that is administered by intravenous, intraportal and intraparenchymal routes to the liver to assess biodistribution using the different routes.

Also, constructs are designed to include a miRNA to knockdown PCSK9 to prevent LDLR and VLDLR degradation and turnover. FIG. 4F shows a construct with a PCSK9-targeting miRNA under control of a promoter that is different from LP1, where the construct can be administered by intravenous, intraportal or intraparenchymal routes to the liver to assess biodistribution and dose range using the different routes. FIG. 4G shows a construct including a LP1-VLDRL transposon and a transposon comprising PCSK9-targeting miRNA under the control of a promoter different from LP1. FIG. 4H shows a construct including a LP1- e-LDLR transposon and a transposon comprising PCSK9-targeting miRNA under the control of a promoter different from LP1.

### Example 2: In Vivo Experiments Using Transgenic Mouse

The experiments in this example involve intravenous, intraportal, and intraparenchymal liver injections of LP1-vldlr and LP1-luciferase constructs into the ldlr -/- mouse to determine dosing, tolerability, biodistribution, safety, and efficacy. Similar experiments in the Yucatan Ldlr -/- transgenic mini-pigs are to be designed to show safety, tolerability and efficacy of the appropriate constructs and administration procedure. Biodistribution, dose-response, pharmacokinetic, pharmacodynamic, safety, and pathological studies may be performed in either the Yucatan pigs or cynomolgus monkeys in a GLP environment.

### Example 3: Determining Transposition Efficacy and Transposase (Ts):Transposon (Tn) Ratio in Liver and Non-Liver Cell Lines

The transposition efficacy of transposons expressing GFP under the control of a constitutive phosphoglycerate kinase (PGK) promoter is determined by fluorescent-activated cell sorting (FACS) analysis of liver and non-liver cell lines (i.e., Huh7, HepG2, HT1801), after transfection with a PGK1-GFP Tn. The constructs include LEAPIN Transposase. FIG. 4A shows an example of a vector that can be used in this example. Various liver-specific promoters are used, including those listed in Table 1 above.

### Example 4: Selecting Liver-Specific Promoters (LSPs)

A liver cell line and a non-liver cell line are selected based on the transposition efficacy and Ts:Tn ratio as determined in Example 3, above. The Green Fluorescent Protein (GFP) expression levels using different liver-specific promoters (Table 1) are determined by co-transfecting the cell lines with transposons (FIGS. 4A and 4B) and transposases. The liver-specific promoters are evaluated as described in Kramer et al. Mol Ther 2003; 7:375-85. GFP expression is determined by FACS analysis after 1 day, 1 week, 2 weeks, and 4 weeks post-transfection.

### Example 5: Evaluating Transposon Constructs Including Human VLDLR, Enhanced LDLR (L339D, K830R, C839A), and Knock-Down of PCSK9

Transfection experiments are performed in liver and non-liver cell lines to evaluate the expression of constructs designed to ameliorate FH caused by LDLR mutations. The two transposon (Tn) construct designs include constructs with human VLDLR, and constructs with enhanced human VLDLR (e-LDLR), having amino acid substitutions (L339D, K830R, and C839A) that are introduced into the coding sequence of human LDLR cDNA to reduce interaction with proprotein convertase subtilisin/kexin type 9 (PCSK9) and inducible degrader of LDLR (IDOL). In addition, constructs are designed to include an miRNA to knockdown PCSK9 to prevent LDLR and VLDLR degradation and turnover, see FIGS. 4F-4H.

The specific cell lines, Ts:Tn ratio, strong constitutive LSPs, and the safest Ts construct (i.e. defined site specific integration) are selected as described in Examples 3 and 4 above. FACS analysis is performed in un-transfected and transfected cells to evaluate LDLR and VLDLR, by methods described previously. See Somanathan et al. Circ Res 2014; 115:591-9; Kozarsky et al. (1996). Nat Genet 3:54-62; Turunen et al. (2016). Mol Ther 2016; 24:620-35.

### Example 6: Generating Transposon (Tn) and Transposase (Ts) Constructs for In Vivo Studies in FH Patient iPSCs and Transgenic ldlr-/- Mice, and Large Animal Models

The transposon (Tn) and transposase (Ts) constructs are identified for in vitro testing in patient’s LDRL -/- stem cells (iPSCs or ESC) and in vivo testing (including luciferase biodistribution) in transgenic ldlr -/- mice. The constructs that are used are schematically shown in FIGS. 4B, 4C, 4D, and 4F. Some of the studies are conducted in large animals, e.g. Yucatan mini-pigs LDRL -/- (Sus scrofa), and non-human primates (cynomolgus monkeys; macaca fascicularis).

### Example 7: mRNA Expression in Huh-7 Cells Transfected With LP1-VLDLR (Hs)/MLT Transposase

An objective of this study was to demonstrate the integration efficiency of the MLT transposase 1 and MLT transposase 2 by generation of two transgenic cell lines - LP1-VLDLR/PGK-GFP and LP1-VLDLR in HUH7 cells.

### Cell Line Generation

HUH7 cells were transfected using certain nucleofection conditions (three different 4D-Nucleofector® Solutions in combination with 15 different Nucleofector® Programs, plus 1 control (no nucleofection)), using two different plasmids (LP1-VLDLR/PGK-GFP and LP1-VLDLR alone) in a combination with an MLT transposase of the present disclosure (SEQ ID NO: 9). After 72 hours, antibiotic treatment (Kan+) was applied to select the positively transfected clones. The cell media including the selection antibiotic was changed every 2-3 days and the cells were visually examined daily for cytotoxicity for 14 days. The genome-editing capability of the MLT transposase was quantified using immunofluorescence detection of the GFP signal after 14 days.

### Cell Line Validation

During the generation and expansion of the cell lines, positive (HEK293) and negative control (HUH7) cell lines were used. Droplet Digital® PCT (ddPCR) was performed to quantify the number of LP1-VLDLR transgene copies per cell. The two positive controls were (1) LP1-VLDLR plasmid alone; and (2) human genomic DNA (gBlocks® Gene Fragments double-stranded DNA fragments of 125-3000 bp in length that are the industry standard for double-stranded DNA). The negative controls were:(1) HEK293 cells; (2) untreated HUH7 cells; (3) HUH7 cells + MLT transposase; and (4) H₂O control.

qPCR was performed to quantify the expression level of the transgene in all cell lines. Three positive controls were used: (1) HEK293 cells; (2) plasmid alone; and (3) gBlocks® Gene Fragments. Three negative controls were used: (1) HUH7 cells; (2) HUH7 cells + MLT transposase; (3) H₂O control. The LP1-VLDLR alone was used as a control for random integration.

### LDL-C Uptake Protocol

Abcam LDL uptake kit (cat.# ab133127) was used according to supplier instructions. Nucleofected HUH7 cells were used at a confluency of 70-80%. The HUH7 cells were stained with LDL-Dylight® 549 for detection of LDL uptake into nucleofected cells. Readout was performed with high content imaging performed using Hoechst staining for the quantification of number of nuclei.

### Reagents Used

Table 2 shows reagents used in the present experiments.

| | |
|---|---|
| Reagents | Supplier |
| LP1-hVLDLR | (VB200724-1074rgj) |
| PGK-GFP | (VB200724-1070ryw) |
| RNA | VB200724-1078hnp (VB200905-1046fxw) |
| RNA | MLT Transposase with hyperactive mutations (VB200926-1055qkq) |
| Huh7 cells | CRL, Leiden, Netherlands |
| HEK293 cells | CRL, Leiden, Netherlands |

### Results

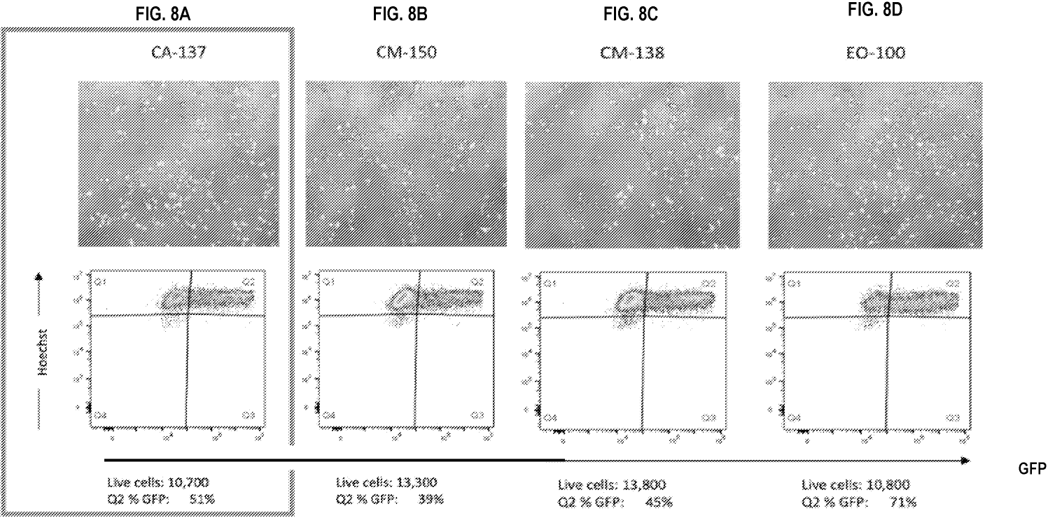

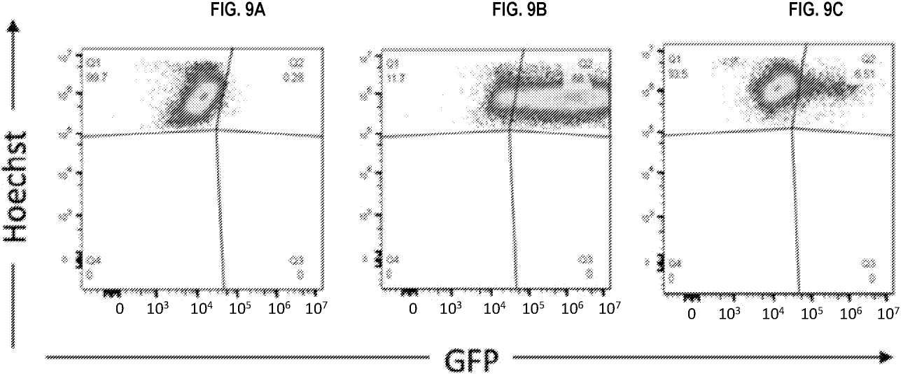

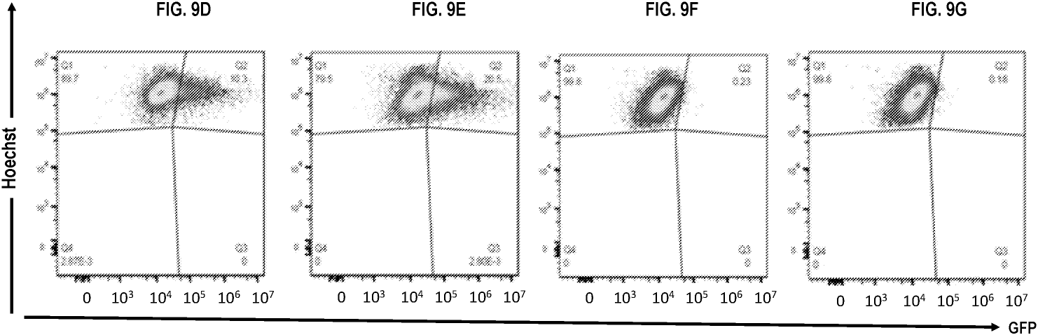

FIG. 6 illustrates GFP expression of Huh7 cells 24 hours post nucleofection; the four panels (from left to right) illustrate data obtained with Nucleofector® Solution kit (Lonza, Basel, Switzerland), Programs CA-137, CM-150, CM-138, and EO-100. The top row of each panel shows GFP fluorescence, and the bottom row shows brightfield microscopy data. FIG. 7 illustrates results of GFP expression of Huh7 cells 4 days post nucleofection; the four panels (from left to right) illustrate data obtained with Nucleofector® Solution kit, Programs CA-137, CM-150, CM-138, and EO-100. The top row of each panel shows GFP fluorescence, and the bottom row shows brightfield microscopy data. FIGS. 8A, 8B, 8D, and 8D illustrate results of quantification of nucleofection efficiency 24 hours post nucleofection: data obtained with Nucleofector®™ Solution kit, Programs CA-137 (FIG. 8A), CM-150 (FIG. 8B), CM-138 (10,700 live cells, Q2 % GFP: 51%) (FIG. 8C), and EO-100 (FIG. 8D). The top row of each figure shows GFP fluorescence, and the bottom row shows FACS plots: (1) 10,700 live cells, Q2 % GFP: 51%; (2) 13,300 live cells, Q2 % GFP: 39%; (3) 13,800 live cells, Q2 % GFP: 45%; and (4) 10,800 live cells, Q2 % GFP: 71%.

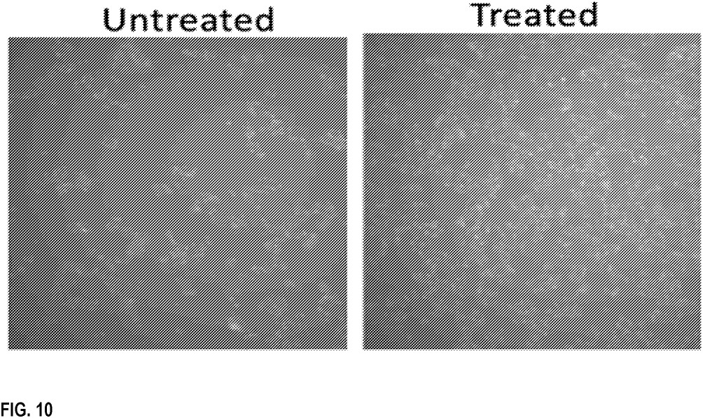

FIGS. 9A, 9B, 9C, 9D, 9E, 9F, and 9G demonstrate FACS plots of transfected Huh7 cells under different conditions, 7 days post transfection, as follows: (FIG. 9A) no program - negative assay control; (FIG. 9B) pmaxGFP - positive assay control; (FIG. 9C) LP1-VLDLR/PGK-GFP plasmid (i.e. a plasmid including a LP1 promoter, a very low-density lipoprotein receptor (VLDLR) gene, and PGK promoter-driven expression of enhanced (eGFP)); (FIG. 9D) LP1-VLDLR/PGK-GFP + MLT 1; (FIG. 9E) LP1-VLDLR/PGK-GFP + MLT 2; (FIG. 9F) MLT 1; and (FIG. 9G) MLT 2. Table 3 below provides information on each of the transfections, for FIGS. 9A through 9G.

TABLE 3

Description of Information in FIGS. 9A through 9G

| | |
|---|---|
| FIG. | Plasmid/RNA |
| 9A | Negative assay control |
| 9B | pmaxGFP - Positive assay control |
| 9C | LP1-VLDLR/PGK-GFP plasmid |
| 9D | LP1-VLDLR/PGK-GFP + MLT transposase 1 (MLT 1) (encoded by SEQ ID NO: 12) |
| 9E | LP1-VLDLR/PGK-GFP + MLT transposase 2 (MLT 2) (encoded by SEQ ID NO: 14) |
| 9F | MLT transposase 1 |
| 9G | MLT transposase 2 |

FACS plots in FIG. 9A, FIG. 9B, FIG. 9C, FIG. 9D, FIG. 9E, FIG. 9F, and FIG. 9G show that both LP1-VLDLR/PGK-GFP + MLT 1 and LP1-VLDLR/PGK-GFP + MLT 2 (panels 4 and 5) both were able to transfect the Huh7 cells effectively when compared to the negative controls in this study (panels 1, 6, and 7). It was observed that the LP1-VLDLR/PGK-GFP without any transposase had expression similar to the expression of the combination of LP1-VLDLR/PGK-GFP and the transposase (MLT 1 or MLT 2). This shows that the presence of the transposase does not negatively impact the viability of the Huh7 cells.

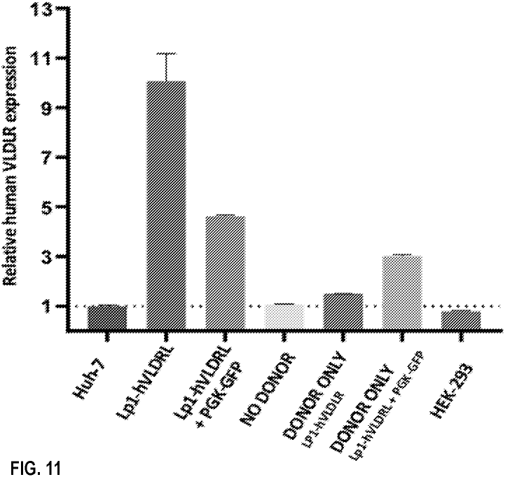

FIG. 10, illustrating LDL-C uptake of untreated Huh7 cells vs. Huh7 cells treated with the VLDLR plasmid and the MLT transposase 2, shows that when Huh7 cells are treated with VLDLR plasmid and the MLT transposase 2, there is about a 30-50% increase in LDL-C uptake for Huh7 cells.

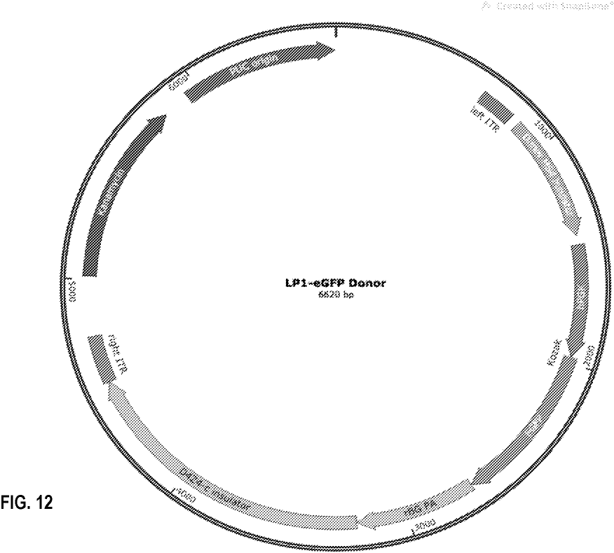

FIG. 11, illustrating qPCR results of VLDLR expression under varying conditions, shows that the presence of the MLT transposase 2 in sample 1 (LP1-hVLDLR) and sample 2 (LP1-hVLDLR + PGK-GFP) increases the VLDLR expression. This demonstrates the dramatic, greater than 10-fold increase in expression of LP1-hVLDLR in human hepatocytes (Huh7 cells) when compared to the controls.

In conclusion, this study has demonstrated, inter alia, the increase in expression of VLDLR mRNA expression when Huh7 cells were treated with LP1-hVLDLR. This study has also demonstrated that LDL-C uptake is increased from about 30% to 50% when treated with VLDLR plasmid and the MLT transposase 2, compared to the untreated cell lines (FIG. 10).

### Example 8: Flow Cytometry Analysis Used to Determine Transposition Activity

This study assessed the use of flow cytometry in order to determine an in vitro transposition of transfected cell lines. In this study, flow cytometry was used in order to measure the presence of GFP in varying hepatocyte and non-hepatocyte cell lines.

Samples were analyzed on a flow cytometer (Becton Dickinson Immunocytometry Systems, BDIS) equipped with a 15 mW air-cooled 488 argon-ion laser. Green GFP fluorescence was collected after a 530/30-nm bandpass (BP) filter. Electronic compensation was used among the fluorescence channels to remove residual spectral overlap. GFP fluorescence data were displayed on a four-decade log scale. The low flow rate setting (12 µL/min) was used for sample acquisition to improve the coefficient of variation on histograms. On each sample, a minimum of 10,000 events was collected within the singlet gate. Analysis of the multivariate data was performed with CELL Questy software (BDIS), and cell cycle analysis of histograms was performed with ModFit LTy software (Verity Software House, Topsham, ME).

Table 4 shows reagents used in the present experiments.

DNA Donor DNA with various promoters
RNA MLT transposase
HT1080 ATCC CCL-121
HepG2 ATCC HB-8065
Huh7 JCRB 0403
CHO ATTC CRL-11965
HEK293 ATTC CRL 1573
ARPE19
661W

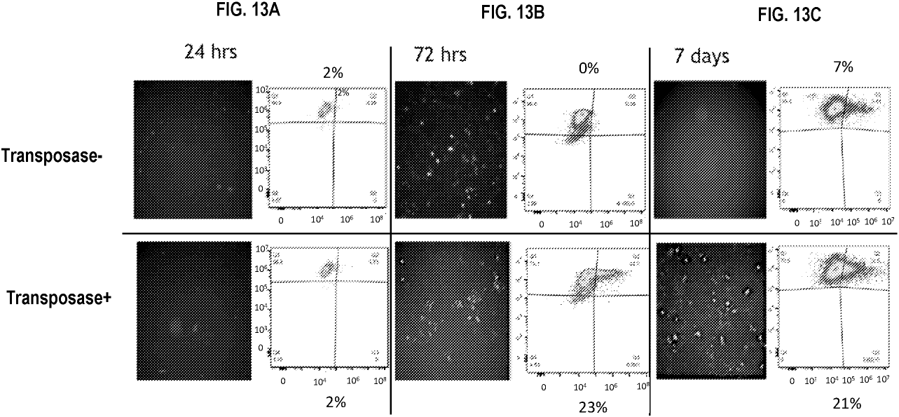

In this study, a DNA donor (plasmid) candidates (FIG. 12) were tested and measured by their GFP expression by flow cytometry in cell lines.

The results of these in vitro pre-clinical discovery studies provide a proof of concept of using transposon constructs to treat human diseases. Also, data obtained in this study supports the use of the flow cytometry to quantitate the number of transposed cells by measuring donor GFP.

### Example 9: Co-Transfection of PGK-GFP and LP1-VLDLR in Huh-7 Human Hepatocytes

An objective of this study was to assess the integration of the VLDLR/PGK-GFP and VLDLR transgenes with and without the MLT transposase 1 and the MLT transposase 2 in accordance with the present disclosure, co-transfected with PGK-GFP and LP1-VLDLR.

### HUH7 Cell Line Preparation

HUH7 Cell Line was tested with 4D-Nucleofector®M Solutions V in combination with 15 different Nucleofector® Programs plus 1 control (no nucleofection). The Nucleofection® Condition with the highest efficiency and lowest mortality was selected after 24 hours for all subsequent experiments.

HUH7 cells were transfected using the previously predetermined nucleofection conditions, using two different plasmids (LP1-VLDLR/PGK-GFP and LP1-VLDLR alone) in a combination with two different transposases - the MLT transposase 1 and the MLT transposase 2. After 72 hours, antibiotic treatment (Kan+) was applied to select the positively transfected clones. Cell media containing the selection antibiotic was changed every 2-3 days and the cells were visually examined daily for cytotoxicity for 14 days. The genome editing capability of the two transposases (MLT1 and MLT 2) was quantified and compared using immunofluorescence detection of the GFP signal after 14 days.

### Assay Optimization

During the generation and expansion of the cell lines, positive (HEK293) and negative control (HUH7) cell lines were used for optimization purposes. Specific Taqman probes for ddPCR assay were designed to detect VLDLR transgene to quantify LP1 VLDLR transgene copy number. Reference gene for Copy Number Determination Assays. VIDLR expression level with Gblock (positive control) and Plasmid-Specific probe (negative control for stable integration.

In Western Blotting and immunocytochemistry (ICC) experiments, up to two antibodies were tested at a two dilutions. The same two antibodies were used to perform ICC in the two cell lines. A single antibody at a specific dilution was selected for further experiments.

LDL uptake was assessed using the LDL Uptake Assay kit (Abcam; cat.# ab133127). Protocol provided by the manufacturer was followed without modification. Cells were plated at up to four different densities. Experiments were performed in technical triplicates. High content imaging and image analysis was performed. Intensity of fluorescent LDL within a cell was calculated.

Assessment of VLDLR expression in the newly generated VLDLR/PGK-GFP and VLDLR cell lines was performed using the Amaxa Nucleofector® Program CA-137 (Lonza). HEK293 cells were used for a positive control, and HUH7 cells were used for negative control. ddPCR was performed to quantify the number of LP1-VLDLR transgene copies per cell, and to quantify the expression level of the transgene in all cell lines. Quantification of VLDLR at protein level was performed by Western blot.

High content imaging and image analysis was performed using Hoechst staining for the quantification of number of nuclei, and immunocytochemistry was performed with the selected antibody against the human VLDLR.

### Reagents

Table 5 summarizes reagents used in the present experiments.

| | |
|---|---|
| Reagents | Supplier |
| DNA-LP1 | LP1-hVLDLR (VB200724-1074rgj) |
| DNA-GFP | PGK-GFP (VB200724-1070ryw) |
| RNA | MLT 1 (VB200905-1046fxw) |
| RNA | MLT 2 (VB200926-1055qkq) |
| Huh7 | CRL, Leiden, Netherlands |
| Nucleofection Reagents | CRL, Leiden, Netherlands |
| TaqMan Probes | CRL, Leiden, Netherlands |
| Antibodies | CRL, Leiden, Netherlands |

### Results

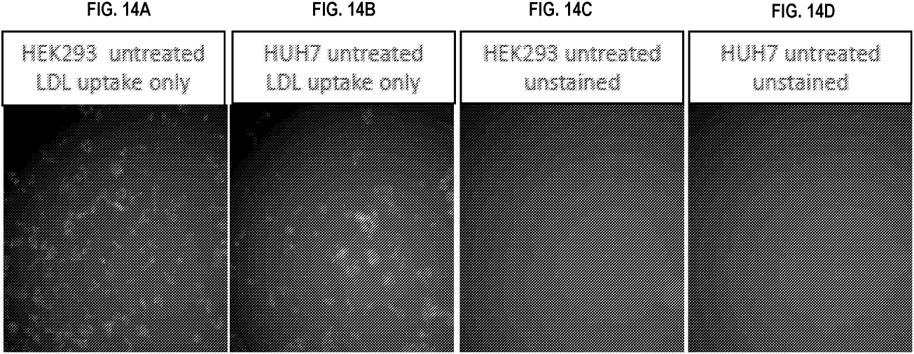

FIGS. 13A, 13B, and 13C are images and FACS plots of GFP expression in Huh7 cells, for assays with (“transposase+”) and without (“transposase-”) the transposase (MLT 1 and MLT 2), which used the gene transfer construct of FIG. 12. FIGS. 13A, 13B, and 13C show data for transposase- assay (top) and transposase+ assay (bottom) at 24 hours (FIG. 13A), 72 hours (FIG. 13B), and 7 days (FIG. 13C), respectively.

The results of the FACs analysis shown in FIGS. 13A, 13B, and 13C were generated using the donor DNA construct of FIG. 12. After 24 hours, in both assays with (transposase+) and without (transposase-) the transposase, there was little to no GFP expression quantified at about 2% for both (FIG. 13A). At 72 hours, in the transposase- transfected cells there was 0% GFP expression, while in the transposase+ cells (transposase + LP1-VLDLR and PGK-GFP) at 72 hours there was 23% GFP expression observed (FIG. 13B). At 7 days, there was 7% GFP expression in the transposase- cells, while there was 21% expression in the transposase+ cells at 72 hours (FIG. 13C). This data was quantified by both ddPCR and FACS.

Accordingly, the data obtained in these experiments illustrates that the transposase + PGK-GFP and LP1-VLDLR can transpose Huh7 cells, as shown in FIGS. 13A, 13B, and 13C. The comparison of these transfected cells demonstrates the significant difference in the transposase- cells and the transposase-+ cells. At 72 hours, the Huh7 cells that were transfected with the transposase + LP1-VLDLR and PGK-GFP displayed 23% GFP expression. This shows that, at 72 hours, the transposase + LP1-VLDLR and PGK-GFP can transfect Huh7 cells without causing detrimental cell death. At 7 days, the transposase+ transfected cells still displayed 21% GFP expression, while the transposase- transfected cells (i.e. no transposase was used in the transfection) displayed 7% GFP expression at 7 days. When comparing this data, it can be seen that, when considering the control GFP % positive cells from the experimental results, it still displays 14% GFP expression which is significant after 7 days.

### Example 10: LDL Uptake in Huh-7 Cells Transposed With LP1-VLDLR

An objective of this study was to assess LDL uptake and regulation at the cellular level in Huh7 cells that were transposed using the MLT transposase of the present disclosure (helper RNA) and a donor DNA construct transfecting cells with human VLDLR (codon optimized) driven by the liver specific promoter, LP1. A goal was to show the successful integration of the VLDLR transgenes in Huh7 cells after two weeks of growth. The kit employs Human LDL. Huh7 cells do not normally express human VLDLR but do express LDLR. It was expected to see an increase in LDL uptake in transposed Huh7 cells expressing VLDLR in addition to LDLR. DyLight® 550 was used as a fluorescent probe for detection of LDL uptake into cultured Hu-7 cells.

### Methodology

The protocol used in the present study was designed for a 96-well plate (for other sizes of plates, the volume of medium/solution to apply to each well was adjusted accordingly):
1. Seed a 96-well plate with 3 x 10⁴ cells/, and grow cells overnight. For HepG2 cells, grow cells for two days before treatment;
2. The next day or third day, treat cells with experimental compounds or vehicle for 24 hours;
3. At the end of the treatment period, replace the culture medium with 75-100 µl/well LDL-DyLight® 550 working solution;
4. Incubate the cells in a 37° C. incubator for an additional three to four hours, or for the period of time used in the experimental protocol;
5. At the end of the LDL uptake incubation, aspirate the culture medium and replace with fresh culture medium or PBS;
6. Examine the degree of LDL uptake under a microscope with filters capable of measuring excitation and emission wavelengths 540 nm and 570 nm, respectively.

### Reagents

Table 6 summarizes reagents used in the present experiments.

| | |
|---|---|
| Reagents | Supplier |
| DNA-LP1 | LP1-hVLDLR (VB200724-1074rgj) (codon optimized) |
| RNA | MLT 1 (VB200905-1046fxw) |
| DNA-GFP | MLT 2 (VB200926-1055qkq) |
| Huh7 | CRL, Leiden, Netherlands |
| Nucleofection Reagents | CRL, Leiden, Netherlands |
| LDL Uptake Assay | Abcam ab133127 - LDL Uptake Assay Kit (Cell-Based) |

### Results

Abcam LDL Uptake Assay Kit (cat.# ab133127) was used according to supplier instructions, to determine LDL uptake in Huh7 cells that were transposed with a transposase (RNA helper) of the present disclosure and LPI-VLDLR donor and maintained in culture for 14 days. Nucleofected HUH7 cells, nucleofected with LP1-VLDLR, were grown for 14 days to a confluency of 70% to 80%. Nucleofected cells were stained with LDL-Dylight® 549 for detection of LDL uptake. Readout was performed with high content imaging.

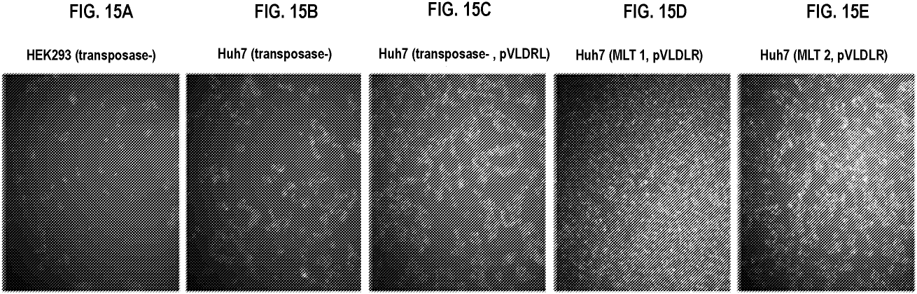

FIGS. 14A, 14B, 14C, and 14D illustrate baseline LDL uptake in non-nucleofected (untreated) HEK293 and Huh7 cells: FIG. 14A shows untreated HEK293 cells, LDL uptake only; FIG. 14B shows untreated Huh7 cells, LDL uptake only; FIG. 14C shows untreated HEK293 cells, unstained; and FIG. 14D shows untreated Huh7 cells, unstained. As shown in FIGS. 14A, 14B, 14C, and 14D, LDL uptake signals were detected in both non-nucleofected (untreated) HEK293 and Huh7 cells stained with LDL-Dylight™ 549 (30% to 50%), as compared to untreated, unstained cells (0%). Both HEK293 and Huh7 cell types have low density lipoprotein receptors (LDLR). Huh7 cells in normoxic conditions do not express very low density lipoprotein receptors (VLDLR), whereas HEK293 express both LDLR and VLDLR.

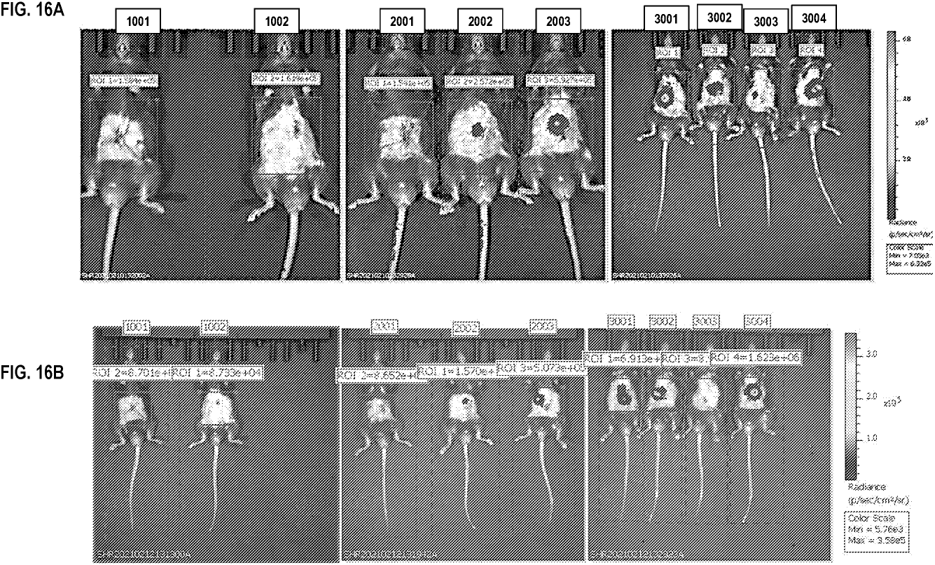

FIGS. 15A, 15B, 15C, 15D, and 15E illustrate LDL uptake in transposed versus non-transposed Huh7 cells: FIG. 15A shows, for comparison, untransposed (transposase -) HEK293 cells; FIG. 15B shows untransposed (transposase -) Huh7 cells; FIG. 15C shows untransposed (transposase -) Huh7 cells, nucleofected with the human LP1-VLDLR plasmid (pVLDLR); FIG. 15D shows transposed with MLT 1 Huh7 cells, nucleofected with the human LP1-VLDLR plasmid (pVLDLR); and FIG. 15E shows transposed with MLT transposase 2 Huh7 cells, nucleofected with the human LP1-VLDLR plasmid (pVLDLR). As shown, LDL uptake signals were smaller in non-nucleofected HEK293 and Huh7 cells, and non-transposed Huh7 cells stained with LDL-Dylight® 549 (30% to 50%), as compared to Huh7 cells transposed with either MLT 1 or the MLT transposase 2 (70% to 90%).

The results suggest that an increase in VLDLR on the surface of transposed Huh7 cells caused an increase in LDL uptake.

### Example 11: Pharmacodynamic Dose Range of LNP LP1-Luciferase/Transposase BLI in Ldlr-/- Mice

An objective of this study was to compare the hepatic uptake of fluorescently labeled lipid nanoparticles in vivo, in wild type (ldlr+/+ and Idlr-/-) C57BL/6 mice of the same background strain. The expression of luciferase was documented by whole body bioluminescence imaging (BLI) at 2 time points (days 3 and 5), after intrahepatic injection of test articles.

This study used the following Lipid Nanoparticle (LNP) formulation: an ionizable lipidoid, cholesterol, a phospholipid, and a PEG-lipid.

### Animal Groups

The present study used purpose-bred, naive mice, strain: ldlr-/- and wild-type (ldlr+/+) C57BL/6J (Jackson Laboratories, Bar Harbor, ME). Number of Males: 16 C57BL/6J wild-type (ldlr+/+) (plus 2 alternates) / 16 ldlr-/-knock- out (plus 2 alternates). Target Age at the initiation of dosing: ~10 wks; and target weight upon arrival: 26.9 + 1.7 g.

The three mice cohorts used in this study are described in Table 7, Table 8, and Table 9.

TABLE 7

Animal Cohort 1.

| Group No. | Test Material | No of animals | Dose level (mg/kg) | Animal serial no. | Route | Conversion factor (uL/gm) | Dose volume (uL) | Dose concentration (mg/ml) | Necropsy day |
|---|---|---|---|---|---|---|---|---|---|
| 1 | Sham (wt ldlr+/+) | 2 | 0 | 1001, 1002 | IH | 1.86 | ~50 | 0 | 22 |
| 2 | LNP high-dose 1 (wt ldlr+/+) | 4 | 1.4 | 2001, 2002, 2003, 2004 | | | | 0.75 | |
| 3 | LNP medium dose 2 (wt ldlr+/+) | 4 | 0.70 | 3001, 3002, 3003, 3004 | | | | 0.375 | |

TABLE 8

Animal Cohort 2

| Group No. | Test Material | No of animals | Dose level (mg/kg) | Animal serial no. | Route | Conversion factor (uL/gm) | Dose volume (uL) | Dose concentration (mg/ml) | Necropsy day |
|---|---|---|---|---|---|---|---|---|---|
| 4 | Sham (Idlr-/-) | 2 | 0 | 4001, 4002 | IH | 1.86 | ~50 | 0 | 12 |
| 5 | LNP high-dose 1 (Idlr-/-) | 4 | 1.4 | 5001, 5002, 5003, 5004 | | | | 0.75 | 7 |
| 6 | LNP medium dose 2 (Idlr-/-) | 4 | 0.70 | 6001, 6002, 6003, 6004 | | | | 0.375 | 33 |

TABLE 9

Animal Cohort 3

| Group No. | Test Material | No of animals | Dose level (mg/kg) | Animal serial no. | Route | Conversion factor (uL/gm) | Dose volume (uL) | Dose concentration (mg/ml) | Necropsy day |
|---|---|---|---|---|---|---|---|---|---|
| 1 | Sham (wt ldlr+/+) | 2 | 0 | 1001, 1002 | IH | 1.86 | ~50 | 0 | 5 |
| 4 | Sham (Idlr-/-) | 2 | 0 | 4001, 4002 | | | | 0 | |
| 7 | LNP low-dose 3 (wt ldlr+/+) | 4 | 0.35 | 7001, 7002, 7003, 7004 | | | | 0.1875 | |
| 8 | LNP low-dose 3 (Idlr-/-) | 4 | 0.35 | 8001, 8002, 8003, 8004 | | | | 0.1875 | 26 |

### Reagents and Test Articles

Table 10 summarizes reagents used in the present experiments.

Reagent
LNP (an ionizable lipidoid, cholesterol, a phospholipid, and a PEG-lipid)
MLT transposase 2
LP1 Luciferase

The vehicle used in the present study was Phosphate-Buffered Saline (PBS), pH 7.4, NO Ca²⁺ or Mg²⁺ (stored at about 4° C.).

The test articles used in the present study were:
· PBS: 1X PBS buffer without Ca or Mg, sterile solution for injection (27G-31G injection needle was used).
· Test Article 1: Empty LNP.
· Test Article 2: LNP LP1- Luciferase 2/MLT transposase 2 (750 ug nucleic acids/mL) (STOCK).

Table 11 summarizes doses of the test articles, wherein that were used in the present experiments.

TABLE 11

Doses of the test articles and PBS

| Dose | STOCK (uL) | PBS (uL) | Concentration (mg/mL) | Total approximate liver dose* (ug) |
|---|---|---|---|---|
| HIGH | 500 | 0 | 0.750 | 37.5 |
| MEDIUM | 250 | 250 | 0.375 | 18.75 |
| LOW | 125 | 500 | 0.1875 | 9.375 |

*Based on ~50 uL injection to the left lateral lobe.

### Results

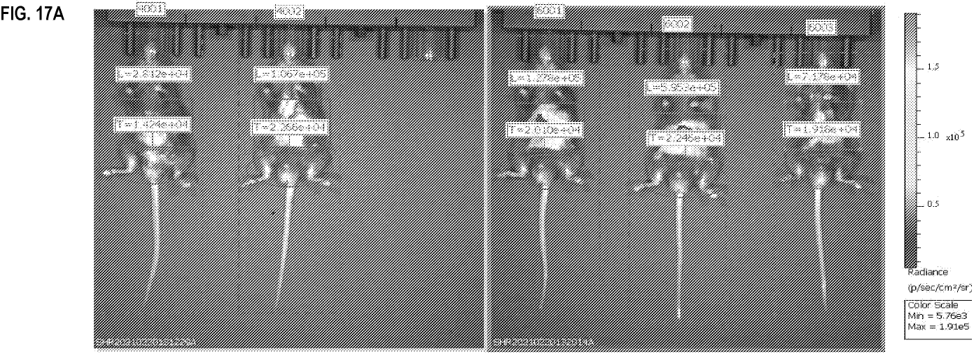

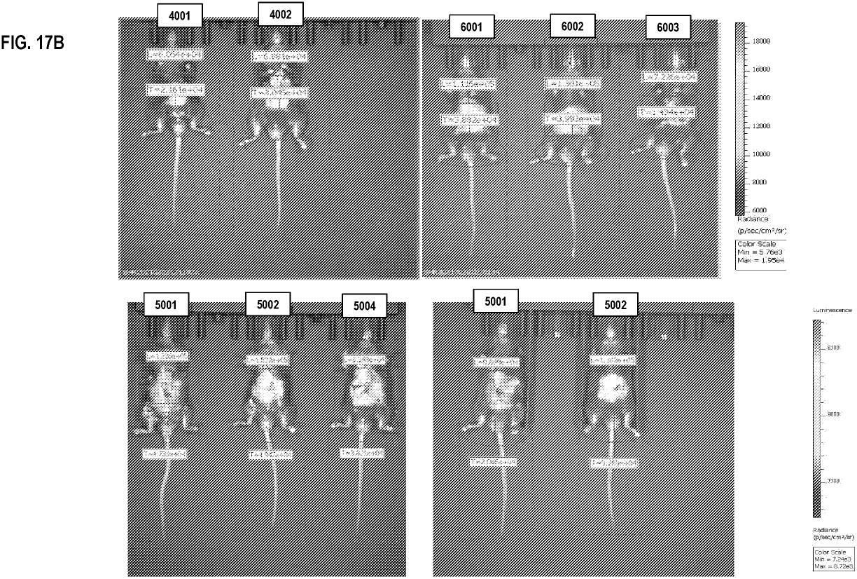

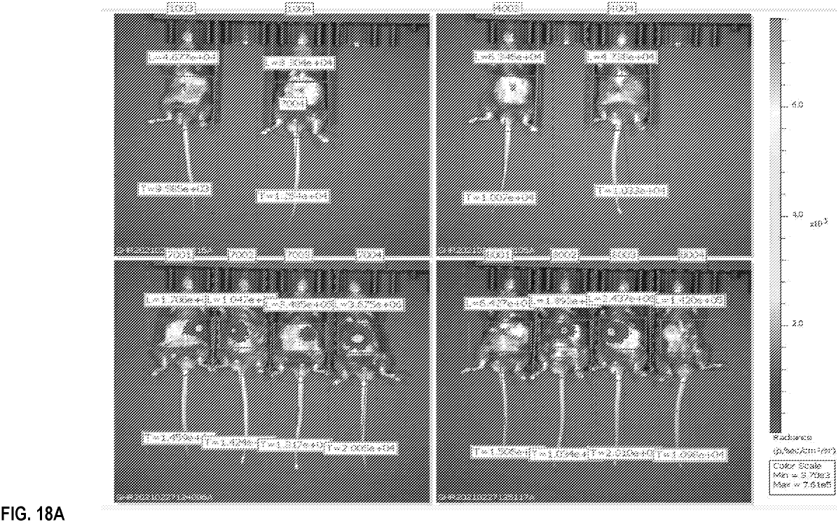

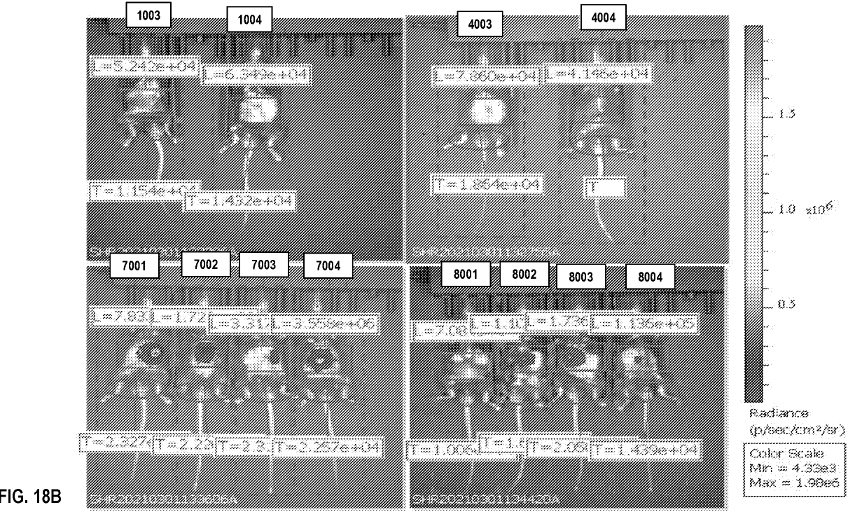

A total of 8 groups of mice were injected either different doses of test article (LP1-vldlr+ MLT transposase) or with empty LNPs (sham surgery). FIGS. 16A and 16B show BLI for cohort 1 on day 3 (FIG. 16A) and day 5 (FIG. 16B). FIGS. 17A and 17B show BLI for cohort 2 on day 3 (FIG. 17A) and day 5 (FIG. 17B). FIGS. 18A and 18B show BLI for cohort 3 on day 3 (FIG. 18A) and day 5 (FIG. 18B). FIG. 19 shows BLI from animals that belong to group 6 and group 8 after a long-term maintenance (26 and 33 days, respectively).

Interestingly, not only wild type mice, but also ldlr-/- mice (groups 5, 6, 7, 8) showed significant uptake of donor DNA, as shown in FIGS. 16A, 16B, 17A, 17B, 18A, 18B, and 19. The empty LNP did not have any lethal effect either on wt or ldlr-/- mice (FIGS. 16A, 16B, 17A, 17B).

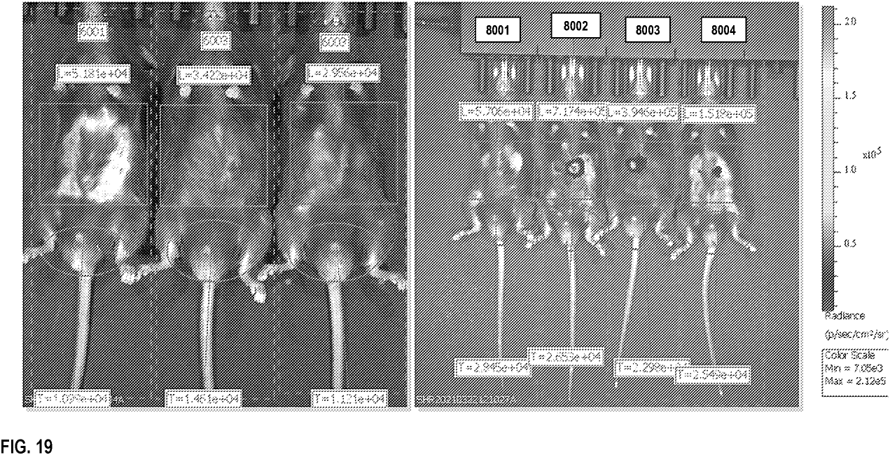

High dose of LNP, although showed successful transposition of donor DNA in wt mice both in 3 and 5 days, but did not show any promising effect in Idlr-/- mice (FIGS. 17A and 17B). However, the high dose of LNP demonstrated a decreasing pattern of luminiscence at day 5 even in wt mice (group 2) (FIG. 17B). Whereas, the medium dose remained effective in wild type (group 3) for both the days but it did not show efficacy in Idlr-/- (group 6) mice on day 5 (FIG. 17B). Interestingly, the low dose showed significant effect on both wt and Idlr-/- mice (FIGS. 18A and 18B). When two groups of animals (group 6 and 8) that received medium and low doses of test articles were allowed to stay longer duration - for 26 and 33 days, the animals with the low dose (group 8) showed continued expression of luminiscence coming from stably integrated luciferase DNA (FIG. 20). One animal from group 2, two animals from group 5, and one animal from 6 died due to the surgical procedure.

In conclusion, the low dose of the test article (0.35 mg/kg) showed the best luminiscence results. The low dosing exhibited the best integration or transposition efficiency for a relatively longer period of time.

### Example 11: Stable Local Liver Integration

The left panel of FIG. 20 shows an Idlr-/- animal administered 50 uL of LNP containing donor DNA alone injected to the left lateral lobe of the liver with the total liver dose of 6.25 ug of DNA and imaged at day 26 post-injection.

The right panel of FIG. 20 shows an Idlr-/- animal administered 50 uL of LNP containing both donor DNA and helper RNA injected to the left lateral lobe of the liver with the total liver dose of 9.375 ug of nucleic acid (DNA 6.25 ug, 3.125 ug RNA) and imaged at day 26 post-injection. The results in FIG. 20 illustrate that when a liver-specific LNP formulation containing LP1-Luciferase 2/MLT transposase is administered by local and targeted injection to the left lateral lobe of the liver in Idlr-/- animals, stable local liver integration of donor DNA occurs after a single dose of the test article. Additionally, there is no evidence of extra-hepatic integration, demonstrating the targeted therapy delivered by the present approach.

### Example 12: Assessment of Safety and Efficacy of Liver-Specific LNP Formulation in Ldlr -/- Mice

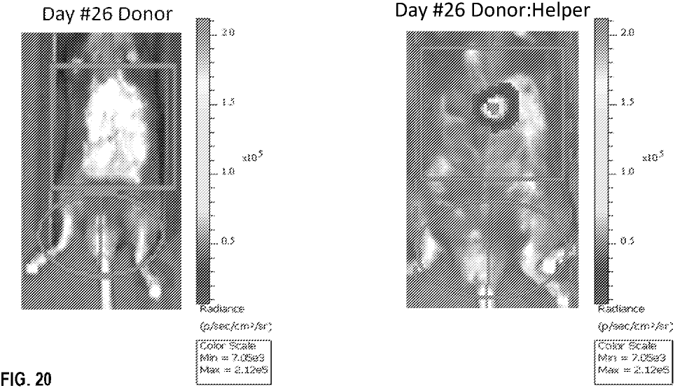

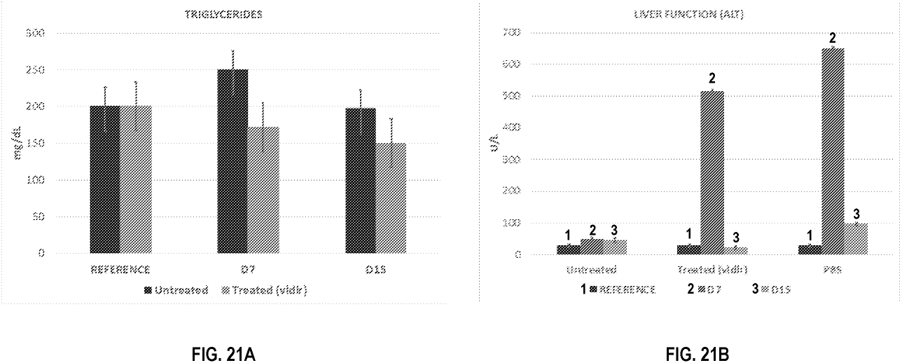

In the present study, Idlr-/- mice were administered the liver-specific LNP formulation using LP1-vldlr/MLT transposase, by direct injection into the left lateral lobe of the liver. FIG. 21A shows the results of the efficacy assessment, illustrating a lowering of triglycerides (mg/dL) compared to the reference range, at days 7 (D7) and 15 (D15), in treated ldlr-/- mice compared to untreated (donor alone) mice. FIG. 21B shows the results of a safety assessment, measuring the liver enzyme, Alanine Aminotransferase (ALT), in the blood of the test animals (in u/L), which is indicative of liver function. In FIG. 21B, the data is shown for the Untreated, Treated and PBS groups, each including data for the Reference (“1”) range, and on D7 (“2”), and D15 (“3”).

These results demonstrate that triglycerides show ~40% lowering compared to the normal reference range. No short-term changes in LDL-C were observed. Also, the initial elevations in ALT (FIG. 21A, day 7) due to the direct liver injection subsequently returns to the normal reference range (FIG. 21A, day 15).

### DEFINITIONS

The following definitions are used in connection with the invention disclosed herein. Unless defined otherwise, all technical and scientific terms used herein have the same meaning as commonly understood to one of skill in the art to which this invention belongs.

The term “in vivo” refers to an event that takes place in a subject’s body.

The term “ex vivo” refers to an event which involves treating or performing a procedure on a cell, tissue and/or organ which has been removed from a subject’s body. Aptly, the cell, tissue and/or organ may be returned to the subject’s body in a method of treatment or surgery.

As used herein, the term “variant” encompasses but is not limited to nucleic acids or proteins which comprise a nucleic acid or amino acid sequence which differs from the nucleic acid or amino acid sequence of a reference by way of one or more substitutions, deletions and/or additions at certain positions. The variant may comprise one or more conservative substitutions. Conservative substitutions may involve, e.g., the substitution of similarly charged or uncharged amino acids.

“Carrier” or “vehicle” as used herein refer to carrier materials suitable for drug administration. Carriers and vehicles useful herein include any such materials known in the art, e.g., any liquid, gel, solvent, liquid diluent, solubilizer, surfactant, or the like, which is nontoxic and which does not interact with other components of the composition in a deleterious manner.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the invention is contemplated. Additional active pharmaceutical ingredients, such as other drugs, can also be incorporated into the described compositions and methods.

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50” covers the range of 45 to 55.

As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features herein set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

What is claimed is:

1. A composition comprising a gene transfer construct, comprising:
(a) a nucleic acid encoding a very low-density lipoprotein receptor (VLDLR) protein or a low-density lipoprotein receptor (LDLR) protein or a functional fragment thereof;
(b) a liver-specific promoter; and
(c) a non-viral vector comprising one or more transposase recognition sites and one or more inverted terminal repeats (ITRs) or end sequences.

2. The composition of claim 1, wherein the gene transfer construct comprises DNA.

3. The composition of claim 1 or 2, wherein the gene transfer construct is codon optimized.

4. The composition of any one of claims 1 to 3, wherein the VLDLR protein is human VLDLR protein, or a functional fragment thereof.

5. The composition of claim 4, wherein the nucleic acid encoding the human VLDLR protein, or the functional fragment thereof comprises a nucleotide sequence encoding the amino acid sequence of SEQ ID NO: 6, or a variant having at least about 90%, or at least about 93%, or at least about 95%, or at least about 97%, or at least about 98% identity thereto.

6. The composition of claim 4, wherein the nucleic acid encoding the human VLDLR protein, or the functional fragment thereof comprises a nucleotide sequence of SEQ ID NO: 4 or a nucleotide sequence of SEQ ID NO: 5, or a variant having at least about 90%, or at least about 93%, or at least about 95%, or at least about 97%, or at least about 98% identity thereto.

7. The composition of any one of claims 1 to 3, wherein the LDLR protein is human LDLR protein, or a functional fragment thereof.

8. The composition of claim 7, wherein the nucleic acid encoding the human LDLR protein, or the functional fragment thereof comprises a nucleotide sequence encoding a protein having an amino acid sequence of SEQ ID NO: 3, or a variant having at least about 90%, or at least about 93%, or at least about 95%, or at least about 97%, or at least about 98% identity thereto.

9. The composition of claim 8, wherein the nucleic acid encoding the human LDLR protein, or the functional fragment thereof comprises a nucleotide sequence of SEQ ID NO: 1, or a variant having at least about 90%, or at least about 93%, or at least about 95%, or at least about 97%, or at least about 98% identity thereto.

10. The composition of claim 7 or 8, wherein the human LDLR protein, or a functional fragment thereof comprises one or more mutations selected from L18F, K830R, and C839A, with reference to the amino acid sequence of SEQ ID NO: 3.

11. The composition of any one of claims 1 to 10, wherein the liver-specific promoter is an LP1 promoter, optionally a human LP1 promoter, or wherein the liver-specific promoter is a promoter of Table 1.

12. The composition of claim 11, wherein the LP1 promoter comprises:
(a) a human antitrypsin promoter, a human ApoE/HCR1 enhancer and/or a SV40 intron; and/or
(b) a nucleic acid sequence of SEQ ID NO: 15, or a variant having at least about 60%, or at least about 70%, or at least about 80%, or at least about 90%, or at least about 93%, or at least about 95%, or at least about 97%, or at least about 98% identity thereto.

13. The composition of any one of claims 1 to 12, wherein the non-viral vector is a DNA plasmid and optionally wherein:
the DNA plasmid comprises one or more insulator sequences that prevent or mitigate activation or inactivation of nearby genes.

14. The composition of any one of claims 1 to 13, wherein:
the ITRs or the end sequences are those of a piggyBac-like transposon, optionally comprising a TTAA repetitive sequence, and/or
the ITRs or the end sequences flank the nucleic acid encoding the VLDLR protein or LDLR protein, or a functional fragment thereof.

15. The composition of any one of claims 1 to 14, further comprising a nucleic acid construct encoding a microRNA that targets pro-protein convertase subtilisin kexin 9 (PCSK9).

16. The composition any one of claims 1 to 15, further comprising a nucleic acid construct encoding a transposase, optionally an mRNA encoding the transposase.

17. The composition of claim 16, wherein the transposase is derived from Myotis lucifugus.

18. The composition of any one of claims 1 to 15, further comprising a nucleic acid construct encoding a transposase, optionally a DNA encoding the transposase.

19. The composition of any one of claims 16 to 18, wherein the transposase is derived from Bombyx mori, Xenopus tropicalis, Trichoplusia ni, Rhinolophus ferrumequinum, Rousettus aegyptiacus, Phyllostomus discolor, Myotis myotis, Myotis lucifugus, Pteropus vampyrus, Pipistrellus kuhlii, Pan troglodytes, Molossus molossus, or Homo sapiens, and/or is an engineered version thereof and/or wherein the transposase specifically recognizes the ITRs or the end sequences.

20. The composition of any one of claims 1 to 19, wherein the composition is in the form of a lipid nanoparticle (LNP).

21. The composition of claim 20, comprising of one or more lipids selected from 1,2-dioleoyl-3-trimethylammonium propane (DOTAP), a cationic cholesterol derivative mixed with dimethylaminoethane-carbamoyl (DC-Chol), phosphatidylcholine (PC), triolein (glyceryl trioleate), and 1,2-distearoyl-sn-glycero-3-phosphoethanolamine-N-[carboxy(polyethylene glycol)-2000] (DSPE-PEG), 1,2-dimyristoyl-rac-glycero-3-methoxypolyethyleneglycol -2000 (DMG-PEG 2 K), and 1,2 distearol -sn-glycerol-3phosphocholine (DSPC) and/or comprising of one or more molecules selected from polyethylenimine (PEI) and poly(lactic-co-glycolic acid) (PLGA), and N-Acetylgalactosamine (GaINAc).

22. The composition of any one of claims 1 to 21, wherein the composition comprises an additional therapeutic agent, wherein the additional therapeutic agent is optionally selected from one or more of a statin, ezetimibe, a bile-acid binding resin, evolocumab, inclisiran, lomitapide and mipomersen, wherein the statin is optionally one or more of Atorvastatin (LIPITOR), fluvastatin (LESCOL), lovastatin (ALTOCOR; ALTOPREV; MEVACOR), pitavastatin (LIVALO), Pravastatin (PRAVACHOL), rosuvastatin calcium (CRESTOR), and simvastatin (ZOCOR).

23. An isolated cell comprising the composition of any one of claims 1 to 21.

24. A method for lowering total cholesterol and/or low-density lipoprotein cholesterol (LDL-C) in a patient, comprising administering to a patient in need thereof a composition of any one of claims 1 to 21.

25. A method for lowering total cholesterol and/or low-density lipoprotein cholesterol (LDL-C) in a patient, comprising:
(a) contacting a cell obtained from a patient with a composition of any one of claims 1 to 21; and
(b) administering the cell to a patient in need thereof.

26. The method of claim 24 or 25, wherein the method improves cardiovascular health of the patient.

27. The method of claim 24 or 25, wherein the method provides greater than about a 40%, or greater than about a 50%, or greater than about a 60%, or greater than about a 70%, or greater than about a 80%, or greater than about a 90% lowering of total cholesterol and/or LDL-C relative to a level of total cholesterol and/or LDL-C without the administration.

28. The method of claim 24 or 25, wherein the method lowers serum LDL-C levels to less than about 500 mg/dL (less than about 13 mmol/L).

29. The method of any one of claims 24 to 28, wherein the method is performed in the absence of a steroid treatment.

30. The method of any one of claims 24 to 29, wherein the method is substantially non-immunogenic.

31. The method of any one of claims 24 to 30, wherein the lowering of total cholesterol and/or LDL-C is durable.

32. The method of any one of claims 24 to 31, wherein the method requires a single administration.

33. The method of any one of claims 24 to 32, wherein the method stimulates and/or increases LDL metabolism in hepatocytes.

34. The method of any one of claims 24 to 33, further comprising administering:
a nucleic acid construct encoding a transposase, optionally derived from Bombyx mori, Xenopus tropicalis, Trichoplusia ni, Rhinolophus ferrumequinum, Rousettus aegyptiacus, Phyllostomus discolor, Myotis myotis, Myotis lucifugus, Pteropus vampyrus, Pipistrellus kuhlii, Pan troglodytes, Molossus molossus, or Homo sapiens, and/or an engineered version thereof; and/or
a nucleic acid construct encoding a microRNA that targets PCSK9.

35. The method of any one of claims 24 to 33, further comprising contacting the cells with:
a nucleic acid construct encoding a transposase, optionally derived from Bombyx mori, Xenopus tropicalis, Trichoplusia ni, Rhinolophus ferrumequinum, Rousettus aegyptiacus, Phyllostomus discolor, Myotis myotis, Myotis lucifugus, Pteropus vampyrus, Pipistrellus kuhlii, Pan troglodytes, Molossus molossus, or Homo sapiens, and/or an engineered version thereof and/or
a nucleic acid construct encoding a microRNA that targets PCSK9.

36. The method of any one of claims 24 to 34, wherein the administering is intravenous.

37. The method of any one of claims 24 to 36, wherein the method treats familial hypercholesterolemia, common hypercholesterolemia, increased triglycerides, insulin resistance, or metabolic syndrome.

38. The method of any one of claims 24 to 34, wherein the administering is to the liver, optionally to the intraportal vein or liver parenchyma.

39. The method of any one of claims 34 to 38, wherein the ratio of the nucleic acid encoding the very low-density lipoprotein receptor protein (VLDLR) or the low-density lipoprotein receptor protein (LDLR), or a functional fragment thereof to the nucleic acid construct encoding transposase is about 5:1, or about 4:1, or about 3:1, or about 2:1, or about 1:1, or about 1:2, or about 1:3, or about 1:4, or about 1:5.

40. The method of any one of claims 34 to 39, wherein the ratio of the nucleic acid encoding the very low-density lipoprotein receptor protein (VLDLR) or low-density lipoprotein receptor protein (LDLR), or a functional fragment thereof to the nucleic acid construct encoding transposase is about 2:1.

41. The method of any one of claims 24 to 40, further comprising administering to the patient in need thereof a miRNA targeting PCSK9.

42. The method of any one of claims 24 to 40, comprising administering to the patient in need thereof an additional therapeutic agent, wherein the additional therapeutic agent is optionally selected from one or more of a statin, ezetimibe, a bile-acid binding resin, evolocumab, inclisiran, lomitapide and mipomersen.

43. The method of claim 42, wherein the statin is one or more of Atorvastatin (LIPITOR), fluvastatin (LESCOL), lovastatin (ALTOCOR; ALTOPREV; MEVACOR), pitavastatin (LIVALO), Pravastatin (PRAVACHOL), rosuvastatin calcium (CRESTOR), and simvastatin (ZOCOR).

44. A method for treating and/or mitigating familial hypercholesterolemia (FH), comprising administering to a patient in need thereof a composition of any one of claims 1 to 22.

45. A method for treating and/or mitigating familial hypercholesterolemia (FH), comprising:
(a) contacting a cell obtained from a patient with a composition of any one of claims 1 to 22; and
(b) administering the cell to a patient in need thereof.

46. The method of claim 44 or 45, wherein the FH is homozygous FH (HoFH) or heterozygous FH (HeFH).

47. The method of any one of claims 44 to 46, wherein the FH is characterized by one or more mutations in one or more of APOB, LDLR, and PCSK9.

48. The method of any one of claims 44 to 47, wherein the method treats and/or mitigates coronary artery disease (CAD).

49. The method of any one of claims 44 to 48, further comprising administering one or more of a statin, ezetimibe, a bile-acid binding resin, evolocumab, inclisiran, lomitapide and mipomersen.

50. The method of any one of claims 44 to 48, wherein the method obviates the need for treatment with one or more of a statin, ezetimibe, a bile-acid binding resin, evolocumab, inclisiran, lomitapide and mipomersen.

51. The method of any one of claims 44 to 50, wherein the method obviates the need for LDL apheresis.

52. The method of any one of claims 44 to 51, wherein the method obviates the need for steroid treatment.

53. The method of any one of claims 44 to 52, wherein the method improves cardiovascular health of the patient.

54. The method of any one of claims 44 to 53, wherein the method is substantially non-immunogenic.

55. The method of any one of claims 44 to 54, wherein the treatment and/or mitigation is durable.

56. The method of any one of claims 44 to 55, wherein the method requires a single administration.

57. The method of any one of claims 44 to 56, wherein the method stimulates and/or increases LDL metabolism in hepatocytes.

58. The method of any one of claims 44 to 57, further comprising administering:
a nucleic acid construct encoding a transposase, optionally derived from Bombyx mori or Xenopus tropicalis and/or an engineered version thereof and/or
a nucleic acid construct encoding a microRNA that targets PCSK9.

59. The method of any one of claims 44 to 58, wherein the administering is intravenous.

60. The method of any one of claims 44 to 59, wherein the administering is to the liver, optionally to the intraportal vein or liver parenchyma.

61. The method of any one of claims 44 to 57, further comprising contacting the cell with a nucleic acid construct encoding a transposase, optionally derived from Bombyx mori, Xenopus tropicalis, Trichoplusia ni, Rhinolophus ferrumequinum, Rousettus aegyptiacus, Phyllostomus discolor, Myotis myotis, Myotis lucifugus, Pteropus vampyrus, Pipistrellus kuhlii, Pan troglodytes, Molossus molossus, or Homo sapiens, and/or an engineered version thereof.

62. The method of claim 58 or claim 61, wherein the ratio of the nucleic encoding the very low-density lipoprotein receptor protein (VLDLR) or the low-density lipoprotein receptor protein (LDLR), or a functional fragment thereof to the nucleic acid construct encoding a transposase is about 5:1, or about 4:1, or about 3:1, or about 2:1, or about 1:1, or about 1:2, or about 1:3, or about 1:4, or about 1:5.

63. The method of claim 58 or claim 61, wherein the ratio of the nucleic acid encoding the very low-density lipoprotein receptor protein (VLDLR) or low-density lipoprotein receptor protein (LDLR), or a functional fragment thereof to the nucleic acid construct encoding the transposase is about 2:1.

64. The method of any one of claims 44 to 63, further comprising administering a miRNA that targets PCSK9.

65. The method of any one of claims 44 to 64, comprising administering to the patient in need thereof an additional therapeutic agent, wherein the additional therapeutic agent is optionally selected from one or more of a statin, ezetimibe, a bile-acid binding resin, evolocumab, lomitapide and mipomersen.

66. The method of claim 65, wherein the statin is one or more of Atorvastatin (LIPITOR), fluvastatin (LESCOL), lovastatin (ALTOCOR; ALTOPREV; MEVACOR), pitavastatin (LIVALO), Pravastatin (PRAVACHOL), rosuvastatin calcium (CRESTOR), and simvastatin (ZOCOR).

67. A composition comprising a gene transfer construct, comprising:
(a) a the nucleic acid encoding a very low-density lipoprotein receptor protein (VLDLR) or a low-density lipoprotein receptor protein (LDLR) or a functional fragment thereof, wherein the VLDLR is human VLDLR that comprises a nucleotide sequence of SEQ ID NO: 4, or a variant of about 90% identity thereto, or a nucleotide sequence of SEQ ID NO: 5, or a variant of about 90% identity thereto;
(b) a liver-specific promoter, wherein the liver-specific promoter is a human LP1 promoter having a nucleic acid sequence of SEQ ID NO: 15, or a variant of having least about 90% identity thereto; and
(c) a non-viral vector comprising one or more transposase recognition sites and one or more inverted terminal repeats (ITRs) or end sequences.


SEQUENCE LISTING (15 sequences)

SEQ ID NO: 1 - DNA, 5173 bp, Artificial Sequence
  Synthetic Sequence
gtgcaatcgcgggaagccagggtttccagctaggacacagcaggtcgtgatccgggtcgggacactgcctggcagaggctgcgagcatggggccctggggctggaaattgcgctggaccgtcgccttgctcctcgccgcggcggggactgcagtgggcgacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggttctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccgacctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtgaacatctgcctggagtcccgtccctgcccagaacccttcctgagacctcgccggccttgttttattcaaagacagagaagaccaaagcattgcctgccagagctttgttttatatatttattcatctgggaggcagaacaggcttcggacagtgcccatgcaatggcttgggttgggattttggtttcttcctttcctcgtgaaggataagagaaacaggcccggggggaccaggatgacacctccatttctctccaggaagttttgagtttctctccaccgtgacacaatcctcaaacatggaagatgaaaggggaggggatgtcaggcccagagaagcaagtggctttcaacacacaacagcagatggcaccaacgggaccccctggccctgcctcatccaccaatctctaagccaaacccctaaactcaggagtcaacgtgtttacctcttctatgcaagccttgctagacagccaggttagcctttgccctgtcacccccgaatcatgacccacccagtgtctttcgaggtgggtttgtaccttccttaagccaggaaagggattcatggcgtcggaaatgatctggctgaatccgtggtggcaccgagaccaaactcattcaccaaatgatgccacttcccagaggcagagcctgagtcactggtcacccttaatatttattaagtgcctgagacacccggttaccttggccgtgaggacacgtggcctgcacccaggtgtggctgtcaggacaccagcctggtgcccatcctcccgacccctacccacttccattcccgtggtctccttgcactttctcagttcagagttgtacactgtgtacatttggcatttgtgttattattttgcactgttttctgtcgtgtgtgttgggatgggatcccaggccagggaaagcccgtgtcaatgaatgccggggacagagaggggcaggttgaccgggacttcaaagccgtgatcgtgaatatcgagaactgccattgtcgtctttatgtccgcccacctagtgcttccacttctatgcaaatgcctccaagccattcacttccccaatcttgtcgttgatgggtatgtgtttaaaacatgcacggtgaggccgggcgcagtggctcacgcctgtaatcccagcactttgggaggccgaggcgggtggatcatgaggtcaggagatcgagaccatcctggctaacacgtgaaaccccgtctctactaaaaatacaaaaaattagccgggcgtggtggcgggcacctgtagtcccagctactcgggaggctgaggcaggagaatggtgtgaacccgggaagcggagcttgcagtgagccgagattgcgccactgcagtccgcagtctggcctgggcgacagagcgagactccgtctcaaaaaaaaaaaacaaaaaaaaaccatgcatggtgcatcagcagcccatggcctctggccaggcatggcgaggctgaggtgggaggatggtttgagctcaggcatttgaggctgtcgtgagctatgattatgccactgctttccagcctgggcaacatagtaagaccccatctcttaaaaaatgaatttggccagacacaggtgcctcacgcctgtaatcccagcactttgggaggctgagctggatcacttgagttcaggagttggagaccaggcctgagcaacaaagcgagatcccatctctacaaaaaccaaaaagttaaaaatcagctgggtacggtggcacgtgcctgtgatcccagctacttgggaggctgaggcaggaggatcgcctgagcccaggaggtggaggttgcagtgagccatgatcgagccactgcactccagcctgggcaacagatgaagaccctatttcagaaatacaactataaaaaaataaataaatcctccagtctggatcgtttgacgggacttcaggttctttctgaaatcgccgtgttactgttgcactgatgtccggagagacagtgacagcctccgtcagactcccgcgtgaagatgtcacaagggattggcaattgtccccagggacaaaacactgtgtcccccccagtgcagggaaccgtgataagcctttctggtttcggagcacgtaaatgcgtccctgtacagatagtggggattttttgttatgtttgcactttgtatattggttgaaactgttatcacttatatatatatatatacacacatatatataaaatctatttatttttgcaaaccctggttgctgtatttgttcagtgactattctcggggccctgtgtagggggttattgcctctgaaatgcctcttctttatgtacaaagattatttgcacgaactggactgtgtgcaacgctttttgggagaatgatgtccccgttgtatgtatgagtggcttctgggagatgggtgtcactttttaaaccactgtatagaaggtttttgtagcctgaatgtcttactgtgatcaattaaatttcttaaatgaaccaa

SEQ ID NO: 2 - DNA, 2583 bp, Artificial Sequence
  Synthetic Sequence
atggggccctggggctggaaattgcgctggaccgtcgccttgctcctcgccgcggcggggactgcagtgggcgacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggttctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccgacctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtga

SEQ ID NO: 3 - protein, 860 aa, Artificial Sequence [sequence omitted]

SEQ ID NO: 4 - DNA, 9213 bp, Artificial Sequence
  Synthetic Sequence
accccgctctccggccgccgccggtgcgggtgctccgctaccggctcctctccgttctgtgctctcttctgctctcggctccccaccccctctcccttccctcctctccccttgcctcccctcctctgcagcgcctgcattattttctgcccgcaggctcggcttgcactgctgctgcagcccggggaggtggctgggtgggtggggaggagactgtgcaagttgtaggggagggggtgccctcttcttccccgctcccttcccccgccaactccttcccctccttctccccctttcccctccccgcccccaccttcttcctcctttcggaaggactggtaacttgtcgtgcggagcgaacggcggcggcggcggcggcggcggcaccatccaggcgggcaccatgggcacgtccgcgctctgggcgctctggctgctgctcgcgctgtgctgggcgccccgggagagcggcgccaccggaaccgggagaaaagccaaatgtgaaccctcccaattccagtgcacaaatggtcgctgtattacgctgttgtggaaatgtgatggggatgaagactgtgttgacggcagtgatgaaaagaactgtgtaaagaagacgtgtgctgaatctgacttcgtgtgcaacaatggccagtgtgttcccagccgatggaagtgtgatggagatcctgactgcgaagatggttcagatgaaagcccagaacagtgccatatgagaacatgccgcatacatgaaatcagctgtggcgcccattctactcagtgtatcccagtgtcctggagatgtgatggtgaaaatgattgtgacagtggagaagatgaagaaaactgtggcaatataacatgtagtcccgacgagttcacctgctccagtggccgctgcatctccaggaactttgtatgcaatggccaggatgactgcagcgatggcagtgatgagctggactgtgccccgccaacctgtggcgcccatgagttccagtgcagcacctcctcctgcatccccatcagctgggtatgcgacgatgatgcagactgctccgaccaatctgatgagtccctggagcagtgtggccgtcagccagtcatacacaccaagtgtccagccagcgaaatccagtgcggctctggcgagtgcatccataagaagtggcgatgtgatggggaccctgactgcaaggatggcagtgatgaggtcaactgtccctctcgaacttgccgacctgaccaatttgaatgtgaggatggcagctgcatccatggcagcaggcagtgtaatggtatccgagactgtgtcgatggttccgatgaagtcaactgcaaaaatgtcaatcagtgcttgggccctggaaaattcaagtgcagaagtggagaatgcatagatatcagcaaagtatgtaaccaggagcaggactgcagggactggagtgatgagcccctgaaagagtgtcatataaacgaatgcttggtaaataatggtggatgttctcatatctgcaaagacctagttataggctacgagtgtgactgtgcagctgggtttgaactgatagataggaaaacctgtggagatattgatgaatgccaaaatccaggaatctgcagtcaaatttgtatcaacttaaaaggcggttacaagtgtgaatgtagtcgtggctatcaaatggatcttgctactggcgtgtgcaaggcagtaggcaaagagccaagtctgatcttcactaatcgaagagacatcaggaagattggcttagagaggaaagaatatatccaactagttgaacagctaagaaacactgtggctctcgatgctgacattgctgcccagaaactattctgggccgatctaagccaaaaggctatcttcagtgcctcaattgatgacaaggttggtagacatgttaaaatgatcgacaatgtctataatcctgcagccattgctgttgattgggtgtacaagaccatctactggactgatgcggcttctaagactatttcagtagctaccctagatggaaccaagaggaagttcctgtttaactctgacttgcgagagcctgcctccatagctgtggacccactgtctggctttgtttactggtcagactggggtgaaccagctaaaatagaaaaagcaggaatgaatggattcgatagacgtccactggtgacagcggatatccagtggcctaacggaattacacttgaccttataaaaagtcgcctctattggcttgattctaagttgcacatgttatccagcgtggacttgaatggccaagatcgtaggatagtactaaagtctctggagttcctagctcatcctcttgcactaacaatatttgaggatcgtgtctactggatagatggggaaaatgaagcagtctatggtgccaataaattcactggatcagagctagccactctagtcaacaacctgaatgatgcccaagacatcattgtctatcatgaacttgtacagccatcaggtaaaaattggtgtgaagaagacatggagaatggaggatgtgaatacctatgcctgccagcaccacagattaatgatcactctccaaaatatacctgttcctgtcccagtgggtacaatgtagaggaaaatggccgagactgtcaaagtactgcaactactgtgacttacagtgagacaaaagatacgaacacaacagaaatttcagcaactagtggactagttcctggagggatcaatgtgaccacagcagtatcagaggtcagtgttcccccaaaagggacttctgccgcatgggccattcttcctctcttgctcttagtgatggcagcagtaggtggctacttgatgtggcggaattggcaacacaagaacatgaaaagcatgaactttgacaatcctgtgtacttgaaaaccactgaagaggacctctccatagacattggtagacacagtgcttctgttggacacacgtacccagcaatatcagttgtaagcacagatgatgatctagcttgacttctgtgacaaatgttgacctttgaggtctaaacaaataatacccccgtcggaatggtaaccgagccagcagctgaagtctctttttcttcctctcggctggaagaacatcaagatacctttgcgtggatcaagcttgtgtacttgaccgtttttatattacttttgtaaatattcttgtccacattctacttcagctttggatgtggttaccgagtatctgtaacccttgaatttctagacagtattgccacctctggccaaatatgcactttccctagaaagccatattccagcagtgaaacttgtgctatagtgtataccacctgtacatacattgtataggccatctgtaaatatcccagagaacaatcactattcttaagcactttgaaaatatttctatgtaaattattgtaaactttttcaatggttgggacaatggcaataggacaaaacgggttactaagatgaaattgccaaaaaaatttataaactaattttgtacgtatgaatgatatctttgacctcaatggaggtttgcaaagactgagtgttcaaactactgtacattttttttcaagtgctaaaaaattaaaccaagcagcttaaccatggtttgtgcctgttcctctaggatgaactcctatcctgagtagtaccaagtgttctaactaaatgtgtgagctgtagttcttgatgttgtataactaagcctgtaattgggctggtttctcttacaccctagcacagttaaaaaatgaagtactgtttaacatttgctcccgaaatatttcttactgtgtaaaagaagctagcttagtctgtacctagaattcctcttggttagaggagtgagtttcttttttttccattaacttgtttcctgatcgagaaacacgtgctaagatttctatgaattctgcttctttatagttaagtctctgttttccatgttctgtcatttatataggtcaaggacaagtgccttctaaggcatggataagggctttcttcttatgaaagtctagactgtcttactgtgaagatggccagggaggatagcaagcaatggcattcttgaacagtctaaggcaggtttctcagtgtggtcctgagaacagaagcatctgcatcacctgggaatgtattagaaatgcaagttcccagccccatcccagacctactgaatcagaaactctgggggcgagggctgaaacctgctttcataagcccttcaggcgatactgatgtaaactaaagcttaaaaaccaccgttttactgtgacttcatgaagaatataattgcaatatcacctaaatttttatcctggctgggatggggcagattcttactatggacaatttctggaacttacagtatgaagagcagatgaggagtttgagacaaagaaaaaattgtacagggagtttagtgaattttccatcaggttggccattactttctttctctaaagtctcaggatgtctggaagcaaggaaagacaaaagacttgaagcaccgggtgcatgctgtgtgtcacaagtgaggtggagagttggctgtggaaggtacagcaattcctctggaattaaatgaccgaccaacacagtaccaagaggatgaagagaagatgttttgcgttgctacgtgaatcagtggatgactcatatttcacttgcatatgaaatataaggcagttaacatcttgtttgtggcttaagcaaggacattaatcctactagatgaaggggaagttggttctgttgtcttaagggatgcccccttgtgattttggtcaactgaatgaaaaagaaaagatagccttggacttttttttcatgggaggtttggaggtcaagcatgtagaaggaaaaggctgtagtcatgaatatagaactgtctgctctccaccttttcctcactctacatttagtcatcacactgaacaggatgtggcctgctgacagtgagtagctgaagtgcctgtttggtaagtaggttagcattcatcatgaactctgaaagccatttcagagtttggctgctgctctgaccctgctgtggtacaaaaaacatggatttttttttttttaactggagtaataccaaattccctttagaagcttgaagtttgatactattgctttccccattataaggattatacagtattaattaattataaggcccaaagtttgttttggcaaataaagtattattctctatcatcatcttttgaattcctgtccctagtatgttctttcatgttctgggtaaaatttccaaccctcaaagagtctaaaacgtctcatttttttttaccacgtcctccccagtaagaagccaaggtacaactcattgtacctacgaggtacaaactcatttgtagaaacaaagtaactagatattcctattgaaataagaatagatatattggtgggccaggcatggtggctcacgcctataatcccagcactttggaaggccaaggcaggaggatcacttgaggccaggagttcgagaccagcctgagcaacatggtgagaccctgtctctacaaaaaaaaaatttataaaaataaaagaaaaaaatagttatatgactaaactactagtcaatacagcttgcaataatagtgcatgaatgataacaggcctcggctttatcacccaaaaaataacttgaattctatgtgaaatgtgttaggataagtttgctctgtctctagagttcaatgtagaagataattaagaggtaaaatatgaagcttatcatacactaaaggagaatatttggagttagatgagcctgtcacacaaagtgaaagtggaccctccagaaattttaggaattgccacaaagattgatgatagatgaataaacagatttgtggcctctggtaaagaaatccagccaagttcctctgtccaaatttttaagtgccctttgcaagctaatctatttggcaaaaaaatagtcccagttgttcaactagctcccttaaaacattctagtaggtgttttaaagcctaggtatcgctgcctctttagttcttgatgaatacaaggcaaaaaaaaaaaaaaaaaaaaaaaaaaaattgaggctttcactttggcatttgcttctgttccttgtatctgtggaagtcaagtagcccttctcaaaaatgagaagccaagctttaaatagcaaactacagggtattaaaagactaactcttcagttaagaacactgtagaaatggtagaccaacttaaagtctaccttttaacaatctttaccccaaacctaaagattctaggggtcacttatctctattacgtaatctaactggaataatttcctagaattcttactagtgaaatcttacccaagtaaaacaatttattattttgaatccaaatgaaagctatactgtttgctttcaatggtggcaaaaacttgcaaaaagcagatatacttttgctaatttaagttgaagtactatgaatacaacctgctaatcttgatccccaaatctgagcccttcagtttcataattgttttatcctgaacgtagttgtgacatagacatttctcaaaagggtctgatacatcaaactcctaacaagtaggagcagcaactagctaaagagtagaaagaagcttcccttattgtgggtgatgaggcggtgcctaaaaaccacatatatataatcccctgggtattacatattctgcaagccctgttagtggggagatggtagtggcaatagcagcgatagttgcgtcagtgctaactgctgctgctgcccgttctgttggcggctgctggccaagtgtgcacacttagccctggttgaggtgcattgtctgctgtcgtctgttaagcatctaaccggagtgggagggcacatcaagcgatttcactctcttaaagaaaaggtttttaggtatttttggtattatatagtctctaaggccagaaggggcttataatgacttctcttctagaattgtaatactaaaaaactactcgagtaaatatcctaacaagtatgatttttcttgggaaaactaaaaattgtacatccatgcttcagccatatcatgggcctaaactatgagagttcagaaatatataaaagtacaaaaatcttcaaaggtacttatttccaaatatggagggaatgaatctgaagagggaacaatggcgtttttcgaacttatttctctctaatccagcctggaggatttttagccgaaacagttgcatgtgaaggattatcagccatctttgcatatccctttcacatctaccttcacacaggaaagctgaatgcattttaccagtgaggctctagatatgaaacttagggatccatagataaataagctactaacaaactgataacagtggtgttccatctctctatcatgtctcctgagtccgtttcacgtttttgagcctggctaatgagaaaaagaacaaagtaggaccatgacccagtgttctcctgtaaatctcactgtaaaaatctcaatgcaaaaacactgagacaggagtgagaccagctgcgggtggggagtttgtgctccgcaggttacctcatggcatatcaaaaatggcttttttcttcactgcgctcaaggaaaaagtcagatgggtgacaaggtggttctgtttagaagcagcaggcaatgtagttaacagcatagacttccgtgtcttgtgtgtggtaccttgggcaagttactaactccctctgtaaagtagagacgatggggactgtctcactgtgacaaggtaagatttaagtgagatgcacacaaaaagctcagcacagtgtctggcatctaatatgtgcagggcagatgtcactactgaatatcacatcaatgctagggctgtatttactgtcacacacaggcaccggtaagtgactttaccggtgaaagtcacaggtacctttatctatgatgaatgtaatgtgttagatggtgaccgaagtgttctccactacacctgtctgaagagatctaagtctgatatttaaggagtgatagcatcttgcatacttaagaaggcttaaaagactgcatggtttaaaccctagaaacataaatggaatgtttctgcctcagttcttttgactacctcctaggacatggaaacaaatatttgggagcctgcatagtctttagatggctgccaaagcacttaaaatctcctttaaattcttaccatatgcttacttttgtactaagtaagtgctcccaagtcttttacttgctcttcacagcaaccctaataggtatctccattttacataggtaaaaactgaggctacaaacaatctatggttccaaatcctatgtgcttactgccgtcttctcttagaaggggatgcagtactgtacaaagattgagctggatgtcgcattgcatgtttgctgagtcctcctcattgctgatgttaggatccttcaacagtttctcagatgccagctatggctggcatccagtatggtggtgtaatggaagtgtactactagcactactacatcaggtcagcacagattaactcagttacgtgttataccttcaagacgctagacattccagaattattttccttggccatccaagtgaagtagaagtagattgttggattgcaaagaaaatgataaagattccctaaaggaatttataagcaggcaacttcattcctatgtataagatattgggagaaaacaaactgtaccacaacaaatcttaagtttcaaggctctcctatagggagaaacatggccaaaaagacaggaagaactgtggcttgtaactcaatgatgctataactccagcactggatcttaagtattctggaatataccagattggtagaactaccagacttgggtatttctcagttaacacacatgcaaggaggaagagagtttttttgaacaaaatgactggctttgacaaaactatcttgctctttttaaatgtcacttgatactagaatgcgatttaaaagcatgtaatgcctcaggagtccggcatatacaaccaaattatcatgctcaggatcttcagaattctaaaggcacatgttagaatgcagagtcctgatgacctgtcaataaacttttttttactaatgaactgtacatttaaataaaagtttatttttgggataa

SEQ ID NO: 5 - DNA, 2622 bp, Artificial Sequence
  Synthetic Sequence
atgggaacttctgcactctgggcattgtggcttctgttggctctgtgctgggcgccacgagaaagcggagctacaggtactggaaggaaggccaagtgtgaacccagtcaattccagtgcacaaacggcaggtgtattactctgctctggaagtgcgacggtgacgaggattgtgtggacggttcagatgagaagaattgcgtcaagaagacctgcgctgaatccgatttcgtctgcaacaacggccagtgtgtgccaagccggtggaaatgtgacggcgatcccgattgcgaagatggcagcgacgagtccccagaacagtgccacatgaggacctgcagaattcatgagatcagctgcggagcccactctactcagtgcattcctgtcagctggcgatgcgatggcgaaaatgactgcgacagcggcgaagacgaagagaactgcgggaacatcacctgttccccggacgaattcacatgttcctccggacgctgtatttccagaaacttcgtttgcaacggccaggatgactgcagcgatgggtctgacgagctggattgcgctcctcctacatgtggtgcacacgaatttcagtgctctaccagtagctgtatacctattagttgggtctgtgacgatgatgccgactgtagtgatcagagtgacgagtctctggaacagtgcggcaggcaaccggtcattcatactaaatgtcccgcgagtgagattcagtgcgggtcaggcgagtgcattcataagaagtggagatgcgatggcgatcctgactgcaaagacggatcagacgaagtgaattgtccttcccggacatgtagacccgaccagtttgaatgtgaagatggaagctgtatacacgggagccgccagtgtaacggaatcagagactgcgtggatggatctgatgaagtcaattgtaagaacgtgaatcaatgccttggccccggaaaattcaaatgcagaagtggggaatgtattgacatatcaaaggtttgcaaccaggaacaagattgccgggattggtcagatgagccacttaaggaatgccatatcaacgaatgtttggtgaacaacgggggctgttcccatatctgtaaagacctggtaataggttacgagtgtgattgcgctgccggatttgaattgatcgacaggaagacctgcggtgacattgatgagtgtcagaatccaggcatctgttcccagatctgtattaacctcaaaggaggatataaatgtgagtgctctaggggatatcagatggacctggcaacaggagtgtgcaaggctgttgggaaagagccctctctgatttttaccaacagacgagacatacgcaagatcggactggaacggaaggagtacatccagctcgtagagcagcttcgaaacactgtcgcgctggacgctgacatagcagcccagaagctgttttgggctgatctgtcccagaaggcaatcttcagcgctagcatcgacgacaaagtggggcgccacgtaaagatgattgataacgtgtataacccagccgcgatcgcggttgactgggtgtataagacaatctactggaccgatgccgcctcaaaaactatctccgtggctaccttggatggaacgaaacgcaagttcttgttcaacagtgatctcagagaacccgcatccatagccgttgaccccctctccggctttgtgtattggtcagattggggcgagcctgcaaagatagagaaggcaggcatgaacggattcgaccggcgacctttggttaccgcagacattcagtggccaaacggcattacactggacctgatcaagagcaggctgtactggctggactctaaactccacatgctttcttccgtagacttgaatggccaggacaggaggatcgtgctgaaaagcctcgagtttctggcacacccgctggcactgaccatctttgaagatcgggtctactggattgatggcgaaaacgaagcggtgtacggggccaataagttcacaggctcagagctggccaccctggtcaataacctgaacgacgcacaggatatcatagtttatcacgagctcgtgcagccaagtgggaagaattggtgtgaggaggacatggaaaatggcggatgtgagtacctctgtctcccagcaccacagatcaacgatcattcaccaaagtacacgtgctcctgtccttccggatataatgtggaggagaatggtagagactgccagtccacagctactaccgtaacgtactctgagacaaaggacaccaacaccaccgaaatttctgctacctccggtttggtgccagggggcatcaacgttaccactgccgtgtctgaagtgtccgtcccacccaagggaacatcagcggcatgggctattctgccgctgctgctgcttgttatggcagctgttggcggatatttgatgtggcggaattggcagcacaaaaatatgaagtcaatgaacttcgataaccctgtctacctcaaaaccacggaggaggatctctctattgatattggtaggcatagcgcttccgtgggtcatacttaccccgccatcagcgtggtgtcaaccgatgatgacctcgcttga

SEQ ID NO: 6 - protein, 873 aa, Artificial Sequence
  Synthetic Sequence
MGTSALWALWLLLALCWAPRESGATGTGRKAKCEPSQFQCTNGRCITLLWKCDGDEDCVDGSDEKNCVKKTCAESDFVCNNGQCVPSRWKCDGDPDCEDGSDESPEQCHMRTCRIHEISCGAHSTQCIPVSWRCDGENDCDSGEDEENCGNITCSPDEFTCSSGRCISRNFVCNGQDDCSDGSDELDCAPPTCGAHEFQCSTSSCIPISWVCDDDADCSDQSDESLEQCGRQPVIHTKCPASEIQCGSGECIHKKWRCDGDPDCKDGSDEVNCPSRTCRPDQFECEDGSCIHGSRQCNGIRDCVDGSDEVNCKNVNQCLGPGKFKCRSGECIDISKVCNQEQDCRDWSDEPLKECHINECLVNNGGCSHICKDLVIGYECDCAAGFELIDRKTCGDIDECQNPGICSQICINLKGGYKCECSRGYQMDLATGVCKAVGKEPSLIFTNRRDIRKIGLERKEYIQLVEQLRNTVALDADIAAQKLFWADLSQKAIFSASIDDKVGRHVKMIDNVYNPAAIAVDWVYKTIYWTDAASKTISVATLDGTKRKFLFNSDLREPASIAVDPLSGFVYWSDWGEPAKIEKAGMNGFDRRPLVTADIQWPNGITLDLIKSRLYWLDSKLHMLSSVDLNGQDRRIVLKSLEFLAHPLALTIFEDRVYWIDGENEAVYGANKFTGSELATLVNNLNDAQDIIVYHELVQPSGKNWCEEDMENGGCEYLCLPAPQINDHSPKYTCSCPSGYNVEENGRDCQSTATTVTYSETKDTNTTEISATSGLVPGGINVTTAVSEVSVPPKGTSAAWAILPLLLLVMAAVGGYLMWRNWQHKNMKSMNFDNPVYLKTTEEDLSIDIGRHSASVGHTYPAISVVSTDDDLA

SEQ ID NO: 7 - DNA, 2622 bp, Artificial Sequence
  Synthetic Sequence
atgggtacttcagcaagatgggcactctggttgctcttggcattgtgttgggcaccaagagatagtggagccacagcttccggcaaaaaggcaaagtgtgacagctcacaatttcagtgcactaacggccggtgcataaccctcctgtggaagtgtgatggggacgaagattgcgctgatggctcagatgagaagaattgtgtgaagaagacctgtgctgagtctgatttcgtgtgtaaaaacggccaatgtgtgccgaataggtggcagtgcgacggagaccccgattgcgaggatggatctgacgagagtccggaacagtgccatatgcgcacctgccggatcaacgagatctcttgtggcgctaggtccacccaatgtatacctgtatcttggcgctgcgatggggaaaatgactgcgacaatggagaggacgaggagaactgcgggaatataacatgtagcgccgatgagttcacatgctctagcggaaggtgtgtgagccgcaattttgtctgtaacgggcaagatgactgtgatgacggatcagacgaattggattgtgctccaccgacttgtggggctcacgaattccagtgtagcacaagctcctgtattccactgagctgggtgtgcgacgacgatgctgactgctctgatcagagcgacgagtcattggaacagtgtgggcgccagccagtgattcacactaagtgccccacatctgaaattcaatgtgggagtggagaatgcattcataagaagtggagatgcgacggggacccagactgtaaagacgggtcagatgaggtgaattgcccaagccgaacatgtcgaccagaccagttcgagtgtgaggatggctcttgcatccacgggagtaggcagtgcaatggaatccgggattgtgtggacggcagcgacgaagtcaactgcaaaaacgtcaatcagtgtctgggccctggaaagttcaaatgccggtccggagagtgtattgacatgtccaaagtgtgcgatcaggagcaggattgtagggattggtccgacgaacctctgaaagagtgtcacatcaatgaatgtctcgtaaacaacggaggctgttcccacatttgcaaagatctcgtaattggttatgaatgcgattgtgctgctggattcgagttgatcgatcggaagacctgcggggatattgatgaatgtcagaatcccggtatctgctcccagatttgcattaatcttaaaggcgggtacaaatgcgagtgttcacgggggtaccagatggatctggctacaggggtgtgcaaggccgttggcaaggaacccagtttgatcttcaccaaccgcagagatattaggaagatcggcttggaacgcaaagagtacattcaactggtggagcaactcaggaacaccgtggccctggacgcagatattgccgctcagaagctgttctgggctgacttgtcccagaaggccatcttctctgccagcatcgacgacaaggtaggcaggcattttaagatgattgacaatgtgtacaacccagctgccatcgctgtggactgggtgtacaagaccatctattggaccgacgctgctagcaaaacaattagtgtggcaactctggacggagccaaacggaagttcctctttaactctgatttgcgggagccagcctccatcgcagtggatccactgtcagggttcgtttattggtccgattggggcgagcctgctaagatagaaaaggctggcatgaacggttttgaccggcggccactcgtcactgaggacatccaatggcccaacggtatcacactggatctcgtgaagagccgcctgtactggctcgatagcaagctccacatgctgtcctccgtggaccttaacggccaggaccgaaggatagttctcaagtccctggagtttctggcacatccacttgcccttacgatatttgaagatagggtgtactggatcgatggagaaaatgaagccgtctacggagctaacaaatttactgggtcagagttggctactcttgttaataacctgaacgacgcccaagacattatcgtgtaccacgagctggtgcagccatccggcaaaaattggtgtgaagatgatatggaaaacggaggctgtgaatacctctgtctgcctgccccccagatcaatgatcattcaccaaagtatacatgcagttgccctaacggctataacctcgaggaaaacgggagggagtgccagagcacctccactccagtgacttattcagagacaaaagatattaatacaacggatatattgaggacatccggcttggttccaggcggaatcaatgttacaactgcagtttcagaggttagtgtaccaccaaagggcacctctgctgcctgggcaatcctgcctcttttgctccttgttatggccgcagtgggaggttacctgatgtggcgcaattggcaacacaagaatatgaagtccatgaatttcgacaaccctgtgtatttgaagacaacagaggaggacctgagtattgatatcggacgccattccgctagtgtcggccatacctacccggctataagtgtggtgtccactgacgacgacctggcctga

SEQ ID NO: 8 - protein, 873 aa, Artificial Sequence
  Synthetic Sequence
MGTSARWALWLLLALCWAPRDSGATASGKKAKCDSSQFQCTNGRCITLLWKCDGDEDCADGSDEKNCVKKTCAESDFVCKNGQCVPNRWQCDGDPDCEDGSDESPEQCHMRTCRINEISCGARSTQCIPVSWRCDGENDCDNGEDEENCGNITCSADEFTCSSGRCVSRNFVCNGQDDCDDGSDELDCAPPTCGAHEFQCSTSSCIPLSWVCDDDADCSDQSDESLEQCGRQPVIHTKCPTSEIQCGSGECIHKKWRCDGDPDCKDGSDEVNCPSRTCRPDQFECEDGSCIHGSRQCNGIRDCVDGSDEVNCKNVNQCLGPGKFKCRSGECIDMSKVCDQEQDCRDWSDEPLKECHINECLVNNGGCSHICKDLVIGYECDCAAGFELIDRKTCGDIDECQNPGICSQICINLKGGYKCECSRGYQMDLATGVCKAVGKEPSLIFTNRRDIRKIGLERKEYIQLVEQLRNTVALDADIAAQKLFWADLSQKAIFSASIDDKVGRHFKMIDNVYNPAAIAVDWVYKTIYWTDAASKTISVATLDGAKRKFLFNSDLREPASIAVDPLSGFVYWSDWGEPAKIEKAGMNGFDRRPLVTEDIQWPNGITLDLVKSRLYWLDSKLHMLSSVDLNGQDRRIVLKSLEFLAHPLALTIFEDRVYWIDGENEAVYGANKFTGSELATLVNNLNDAQDIIVYHELVQPSGKNWCEDDMENGGCEYLCLPAPQINDHSPKYTCSCPNGYNLEENGRECQSTSTPVTYSETKDINTTDILRTSGLVPGGINVTTAVSEVSVPPKGTSAAWAILPLLLLVMAAVGGYLMWRNWQHKNMKSMNFDNPVYLKTTEEDLSIDIGRHSASVGHTYPAISVVSTDDDLA

SEQ ID NO: 9 - protein, 572 aa, Artificial Sequence
  Synthetic Sequence
MAQHSDYSDDEFCADKLSNYSCDSDLENASTSDEDSSDDEVMVRPRTLRRRRISSSSSDSESDIEGGREEWSHVDNPPVLEDFLGHQGLNTDAVINNIEDAVKLFIGDDFFEFLVEESNRYYNQNRNNFKLSKKSLKWKDITPQEMKKFLGLIVLMGQVRKDRRDDYWTTEPWTETPYFGKTMTRDRFRQIWKAWHFNNNADIVNESDRLCKVRPVLDYFVPKFINIYKPHQQLSLDEGIVPWRGRLFFRVYNAGKIVKYGILVRLLCESDTGYICNMEIYCGEGKRLLETIQTVVSPYTDSWYHIYMDNYYNSVANCEALMKNKFRICGTIRKNRGIPKDFQTISLKKGETKFIRKNDILLQVWQSKKPVYLISSIHSAEMEESQNIDRTSKKKIVKPNALIDYNKHMKGVDRADQYLSYYSILRRTVKWTKRLAMYMINCALFNSYAVYKSVRQRKMGFKMFLKQTAIHWLTDDIPEDMDIVPDLQPVPSTSGMRAKPPTSDPPCRLSMDMRKHTLQAIVGSGKKKNILRRCRVCSVHKLRSETRYMCKFCNIPLHKGACFEKYHTLKNY

SEQ ID NO: 10 - DNA, 1719 bp, Artificial Sequence
  Synthetic Sequence
atggcccagcacagcgactacagcgacgacgagttctgtgccgataagctgagtaactacagctgcgacagcgacctggaaaacgccagcacatccgacgaggacagctctgacgacgaggtgatggtgcggcccagaaccctgagacggagaagaatcagcagctctagcagcgactctgaatccgacatcgagggcggccgggaagagtggagccacgtggacaaccctcctgttctggaagattttctgggccatcagggcctgaacaccgacgccgtgatcaacaacatcgaggatgccgtgaagctgttcataggagatgatttctttgagttcctggtcgaggaatccaaccgctattacaaccagaatagaaacaacttcaagctgagcaagaaaagcctgaagtggaaggacatcacccctcaggagatgaaaaagttcctgggactgatcgttctgatgggacaggtgcggaaggacagaagggatgattactggacaaccgaaccttggaccgagaccccttactttggcaagaccatgaccagagacagattcagacagatctggaaagcctggcacttcaacaacaatgctgatatcgtgaacgagtctgatagactgtgtaaagtgcggccagtgttggattacttcgtgcctaagttcatcaacatctataagcctcaccagcagctgagcctggatgaaggcatcgtgccctggcggggcagactgttcttcagagtgtacaatgctggcaagatcgtcaaatacggcatcctggtgcgccttctgtgcgagagcgatacaggctacatctgtaatatggaaatctactgcggcgagggcaaaagactgctggaaaccatccagaccgtcgtttccccttataccgacagctggtaccacatctacatggacaactactacaattctgtggccaactgcgaggccctgatgaagaacaagtttagaatctgcggcacaatcagaaaaaacagaggcatccctaaggacttccagaccatctctctgaagaagggcgaaaccaagttcatcagaaagaacgacatcctgctccaagtgtggcagtccaagaaacccgtgtacctgatcagcagcatccatagcgccgagatggaagaaagccagaacatcgacagaacaagcaagaagaagatcgtgaagcccaatgctctgatcgactacaacaagcacatgaaaggcgtggaccgggccgaccagtacctgtcttattactctatcctgagaagaacagtgaaatggaccaagagactggccatgtacatgatcaattgcgccctgttcaacagctacgccgtgtacaagtccgtgcgacaaagaaaaatgggattcaagatgttcctgaagcagacagccatccactggctgacagacgacattcctgaggacatggacattgtgccagatctgcaacctgtgcccagcacctctggtatgagagctaagcctcccaccagcgatcctccatgtagactgagcatggacatgcggaagcacaccctgcaggccatcgtcggcagcggcaagaagaagaacatccttagacggtgcagggtgtgcagcgtgcacaagctgcggagcgagactcggtacatgtgcaagttttgcaacattcccctgcacaagggagcctgcttcgagaagtaccacaccctgaagaattactag

SEQ ID NO: 11 - DNA, 1719 bp, Artificial Sequence
  Synthetic Sequence
atggcccagcacagcgactacagcgacgacgagttctgtgccgataagctgagtaactacagctgcgacagcgacctggaaaacgccagcacatccgacgaggacagctctgacgacgaggtgatggtgcggcccagaaccctgagacggagaagaatcagcagctctagcagcgactctgaatccgacatcgagggcggccgggaagagtggagccacgtggacaaccctcctgttctggaagattttctgggccatcagggcctgaacaccgacgccgtgatcaacaacatcgaggatgccgtgaagctgttcataggagatgatttctttgagttcctggtcgaggaatccaaccgctattacaaccagaagagaaacaacttcaagctgagcaagaaaagcctgaagtggaaggacatcacccctcaggagatgaaaaagttcctgggactgatcgttctgatgggacaggtgcggaaggacagaagggatgattactggacaaccgaaccttggaccgagaccccttactttggcaagaccatgaccagagacagattcagacagatctggaaagcctggcacttcaacaacaatgctgatatcgtgaacgagtctgatagactgtgtaaagtgcggccagtgttggattacttcgtgcctaagttcatcaacatctataagcctcaccagcagctgagcctggatgaaggcatcgtgccctggcggggcagactgttcttcagagtgtacaatgctggcaagatcgtcaaatacggcatcctggtgcgccttctgtgcgagagcgatacaggctacatctgtaatatggaaatctactgcggcgagggcaaaagactgctggaaaccatccagaccgtcgtttccccttataccgacagctggtaccacatctacatggacaactactacaattctgtggccaactgcgaggccctgatgaagaacaagtttagaatctgcggcacaatcagaaaaaacagaggcatccctaaggacttccagaccatctctctgaagaagggcgaaaccaagttcatcagaaagaacgacatcctgctccaagtgtggcagtccaagaaacccgtgtacctgatcagcagcatccatagcgccgagatggaagaaagccagaacatcgacagaacaagcaagaagaagatcgtgaagcccaatgctctgatcgactacaacaagcacatgaaaggcgtggaccgggccgaccagtacctgtcttattactctatcctgagaagaacagtgaaatggaccaagagactggccatgtacatgatcaattgcgccctgttcaacagctacgccgtgtacaagtccgtgcgacaaagaaaaatgggattcaagatgttcctgaagcagacagccatccactggctgacagacgacattcctgaggacatggacattgtgccagatctgcaacctgtgcccagcacctctggtatgagagctaagcctcccaccagcgatcctccatgtagactgagcatggacatgcggaagcacaccctgcaggccatcgtcggcagcggcaagaagaagaacatccttagacggtgcagggtgtgcagcgtgcacaagctgcggagcgagactcggtacatgtgcaagttttgcaacattcccctgcacaagggagcctgcttcgagaagtaccacaccctgaagaattactag

SEQ ID NO: 12 - protein, 572 aa, Artificial Sequence
  Synthetic Sequence
MAQHSDYSDDEFCADKLSNYSCDSDLENASTSDEDSSDDEVMVRPRTLRRRRISSSSSDSESDIEGGREEWSHVDNPPVLEDFLGHQGLNTDAVINNIEDAVKLFIGDDFFEFLVEESNRYYNQKRNNFKLSKKSLKWKDITPQEMKKFLGLIVLMGQVRKDRRDDYWTTEPWTETPYFGKTMTRDRFRQIWKAWHFNNNADIVNESDRLCKVRPVLDYFVPKFINIYKPHQQLSLDEGIVPWRGRLFFRVYNAGKIVKYGILVRLLCESDTGYICNMEIYCGEGKRLLETIQTVVSPYTDSWYHIYMDNYYNSVANCEALMKNKFRICGTIRKNRGIPKDFQTISLKKGETKFIRKNDILLQVWQSKKPVYLISSIHSAEMEESQNIDRTSKKKIVKPNALIDYNKHMKGVDRADQYLSYYSILRRTVKWTKRLAMYMINCALFNSYAVYKSVRQRKMGFKMFLKQTAIHWLTDDIPEDMDIVPDLQPVPSTSGMRAKPPTSDPPCRLSMDMRKHTLQAIVGSGKKKNILRRCRVCSVHKLRSETRYMCKFCNIPLHKGACFEKYHTLKNY

SEQ ID NO: 13 - DNA, 1719 bp, Artificial Sequence
  Synthetic Sequence
atggcccagcacagcgactaccccgacgacgagttcagagccgataagctgagtaactacagctgcgacagcgacctggaaaacgccagcacatccgacgaggacagctctgacgacgaggtgatggtgcggcccagaaccctgagacggagaagaatcagcagctctagcagcgactctgaatccgacatcgagggcggccgggaagagtggagccacgtggacaaccctcctgttctggaagattttctgggccatcagggcctgaacaccgacgccgtgatcaacaacatcgaggatgccgtgaagctgttcataggagatgatttctttgagttcctggtcgaggaatccaaccgctattacaaccagaatagaaacaacttcaagctgagcaagaaaagcctgaagtggaaggacatcacccctcaggagatgaaaaagttcctgggactgatcgttctgatgggacaggtgcggaaggacagaagggatgattactggacaaccgaaccttggaccgagaccccttactttggcaagaccatgaccagagacagattcagacagatctggaaagcctggcacttcaacaacaatgctgatatcgtgaacgagtctgatagactgtgtaaagtgcggccagtgttggattacttcgtgcctaagttcatcaacatctataagcctcaccagcagctgagcctggatgaaggcatcgtgccctggcggggcagactgttcttcagagtgtacaatgctggcaagatcgtcaaatacggcatcctggtgcgccttctgtgcgagagcgatacaggctacatctgtaatatggaaatctactgcggcgagggcaaaagactgctggaaaccatccagaccgtcgtttccccttataccgacagctggtaccacatctacatggacaactactacaattctgtggccaactgcgaggccctgatgaagaacaagtttagaatctgcggcacaatcagaaaaaacagaggcatccctaaggacttccagaccatctctctgaagaagggcgaaaccaagttcatcagaaagaacgacatcctgctccaagtgtggcagtccaagaaacccgtgtacctgatcagcagcatccatagcgccgagatggaagaaagccagaacatcgacagaacaagcaagaagaagatcgtgaagcccaatgctctgatcgactacaacaagcacatgaaaggcgtggaccgggccgaccagtacctgtcttattactctatcctgagaagaacagtgaaatggaccaagagactggccatgtacatgatcaattgcgccctgttcaacagctacgccgtgtacaagtccgtgcgacaaagaaaaatgggattcaagatgttcctgaagcagacagccatccactggctgacagacgacattcctgaggacatggacattgtgccagatctgcaacctgtgcccagcacctctggtatgagagctaagcctcccaccagcgatcctccatgtagactgagcatggacatgcggaagcacaccctgcaggccatcgtcggcagcggcaagaagaagaacatccttagacggtgcagggtgtgcagcgtgcacaagctgcggagcgagactcggtacatgtgcaagttttgcaacattcccctgcacaagggagcctgcttcgagaagtaccacaccctgaagaattactag

SEQ ID NO: 14 - protein, 572 aa, Artificial Sequence
  Synthetic Sequence
MAQHSDYPDDEFRADKLSNYSCDSDLENASTSDEDSSDDEVMVRPRTLRRRRISSSSSDSESDIEGGREEWSHVDNPPVLEDFLGHQGLNTDAVINNIEDAVKLFIGDDFFEFLVEESNRYYNQNRNNFKLSKKSLKWKDITPQEMKKFLGLIVLMGQVRKDRRDDYWTTEPWTETPYFGKTMTRDRFRQIWKAWHFNNNADIVNESDRLCKVRPVLDYFVPKFINIYKPHQQLSLDEGIVPWRGRLFFRVYNAGKIVKYGILVRLLCESDTGYICNMEIYCGEGKRLLETIQTVVSPYTDSWYHIYMDNYYNSVANCEALMKNKFRICGTIRKNRGIPKDFQTISLKKGETKFIRKNDILLQVWQSKKPVYLISSIHSAEMEESQNIDRTSKKKIVKPNALIDYNKHMKGVDRADQYLSYYSILRRTVKWTKRLAMYMINCALFNSYAVYKSVRQRKMGFKMFLKQTAIHWLTDDIPEDMDIVPDLQPVPSTSGMRAKPPTSDPPCRLSMDMRKHTLQAIVGSGKKKNILRRCRVCSVHKLRSETRYMCKFCNIPLHKGACFEKYHTLKNY

SEQ ID NO: 15 - DNA, 545 bp, Artificial Sequence
  Synthetic Sequence
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttgtgtattttagattccaacctatggaactga